(19) United States
(12) Patent Application Publication
(10) Pub. No.: US 2011/0159520 A1
(43) Pub. Date: Jun. 30, 2011

(54) HYBRID PROTEINS COMPRISING MEMBRANE RECEPTOR AND ION CHANNEL, AND THEIR USE AS BIOSENSORS

(75) Inventors: Michel Vivaudou, Saint-egreve (FR); Christophe Moreau, Saint Nizier Du Moucherotte (FR); Jean Revilloud, Saint Martin Le Vinoux (FR); Julien Dupuis, Grenoble (FR)

(73) Assignee: UNIVERSITE JOSEPH FOURIER, Grenoble Cedex 09 (FR)

(21) Appl. No.: 12/997,649

(22) PCT Filed: Jun. 12, 2009
(86) PCT No.: PCT/EP2009/057264

§ 371 Date: Mar. 4, 2011

(30) Foreign Application Priority Data
Jun. 12, 2008 (EP) 08290547.2

(51) Int. Cl. G01N 27/26; C07K 19/00; C07H 21/04; G01N 33/53

(52) U.S. Cl. 435/7.2; 530/350; 536/23.1; 436/149

(57) ABSTRACT

The present invention relates to the use of a hybrid protein including the sequence of a first membrane receptor fused at its C-terminus to the N-terminus of an ion channel, and possibly containing a linker between the C-terminus of the first membrane receptor and the N-terminus part of the ion channel, the linker being absent in the natural configuration of the first membrane receptor and the ion channel, as a biosensor for: the screening of drugs modulating the activity of the first membrane receptor in its natural configuration, and/or for the in vitro diagnosis of pathologies associated with the presence or the variation of amount of a molecule modifying the activity of the first membrane receptor in its natural configuration.

The present invention relates to hybrid proteins comprising membrane receptor and ion channel, and their use as biosensors.

Due to their ability to selectively bind ligands with high affinity, membrane proteins (receptors, transporters) represent, in theory, biochemical probes (bioprobes) or biochemical sensors (biosensors) useful for the detection and the screening of various molecules. The use of biosensors covers numerous areas including environmental safety and food quality (detection of pollutants/contaminants), as well as human health such as medical diagnostics (detection of microorganisms, toxics) and therapeutics (drug discovery).

However, in practice, the use of biosensors to detect molecules of interest is limited due to the lack of sensitivity and the difficulties to prepare said biosensors.

Detection of ligand binding requires high amounts of purified proteins which are particularly difficult to obtain with membrane proteins, and the detection of the molecules of interest requires highly sensitive biosensors since most of the events involving hazards in human health (i.e. airborne droplets/dust transmission routes), in environmental safety and in food quality occur when causative agents are at very low concentration (down to attomolar (10⁻¹⁸M) to zeptomolar (10⁻²¹M) or even below).

Ion channels are cellular membrane proteins that mediate a wide variety of physiological functions including rapid signaling, excitability and transport [Hille B (2001) Ionic channels of excitable membranes, 3^{rd} edition, Sinauer Associates, Sunderland, M A]. They represent highly sensitive electrical sensors since the current pulses of single channels which are of the order of a few picoamperes lasting a few milliseconds, are yet detectable by conventional electrophysiological techniques. Ion-channel based biosensors are attractive because of the electrical nature of the signal, the measurable currents produced by a single channel that enable single-molecule detection, and the ability to function in the physiological context of a liquid environment.

Engineering of ion-channel derived biosensors has relied on the design of synthetic ion-conducting pores gated by simple chemical ligands [Bayley H, Jayasinghe L (2004) Functional engineered channels and pores. Mol Membr Biol. 21:209-20; Gorteau V, Bollot G, Mareda J, Pasini D, Tran D H, Lazar A N, Coleman A W, Sakai N, Matile S (2005)Synthetic multifunctional pores that open and close in response to chemical stimulation. Bioorg Med. Chem. 13:5171-80]More recent, versatile, designs consist of hybrid proteins associating a natural ion channel with the ligand binding domain of a biological receptor [Bouzat C, Gumilar F, Spitzmaul G, Wang H L, Rayes D, Hansen S B, Taylor P, Sine S M (2004) Coupling of agonist binding to channel gating in an ACh-binding protein linked to an ion channel. Nature. 430:896-900; Grutter T, Prado de Carvalho L, Dufresne V, Taly A, Fischer M, Changeux J P (2005)A chimera encoding the fusion of an acetylcholine-binding protein to an ion channel is stabilized in a state close to the desensitized form of ligand-gated ion channels. C R Biol. 328:223-34; Ohndorf U M, MacKinnon R (2005) Construction of a cyclic nucleotide-gated KcsA K⁺ channel. J Mol. Biol. 350:857-65].

US2005/063989 discloses hybrid proteins comprising ion channel Kir6.2 preferably fused to ABC transporters. SUR/Kir6.2, one of the preferred proteins of this document, is able to generate an electrical signal after stimulation of SUR part contained in the hybrid protein. However, this document mentions neither that modifications in the ion channel sequences can enhance the electric signal generation, nor the ability to develop a new membrane receptor biosensor by using other membrane receptors which, unlike SUR with Kir6.2, are not naturally associated with an ion channel.

Therefore, there is a need for new biosensors which are more sensitive and easier to prepare.

One of the aims of the invention is to provide a hybrid protein comprising a membrane receptor and an ion channel, which is very sensitive, easy to prepare and wherein the membrane receptor retains its ability to interact with its ligand and the ion channel retains its ability to generate and regulate flux of ions that produce an electrical current.

Another aim of the invention is to provide a hybrid protein that can be used in cell-free and whole-cell conditions and wherein the membrane receptor could be modified to interact with specific ligands.

The invention relates to the use of a hybrid protein comprising the sequence of a first membrane receptor covalently fused at its C-terminus to the N-terminus of a ion channel, and possibly containing a linker between the C-terminus of said first membrane receptor and the N-terminus part of said ion channel, said linker being absent in the natural configuration of said first membrane receptor and said ion channel,
said ion channel sequence being deleted of a number of amino acids ranging from 1 to the total number of amino acids of the region extending from the first amino acid at the N-terminus part of said ion channel to the first amino acids of the first α-helix of the transmembrane domain of said ion channel, possibly containing a tag sequence,
said first membrane receptor being liable to present in its extracellular domain a mutation allowing the specific interaction with a ligand different from the ligand that interacts with the first membrane receptor in its natural configuration,
said first membrane receptor being liable to present in its cytoplasmic tail, said cytoplasmic tail being a sequence delimited by the first amino acid after the last amino acid of the transmembrane helix and the last amino acid of said first membrane receptor,
   a deletion of a number of amino acids ranging from 1 to the total number of amino acids of the region delimited by the cytoplasmic tail, preferably of a number of amino acids ranging from 1 to 20, preferably of a number of amino acids ranging from 1 to 15, more preferably of a number of amino acids ranging from 1 to 10, amino acids at the C-terminus, and/or
   an addition, in particular after the last amino acid at the C-terminus of said first membrane receptor, of an additional sequence of a number of amino acids ranging from 1 to the total number of amino acids of the region delimited by the cytoplasmic tail, preferably of a number of amino acids ranging from 1 to 20 amino acids, preferably of a number of amino acids ranging from 1 to 15 amino acids, more preferably of a number of amino acids ranging from 1 to 10 amino acids, preferably contiguous, originating from a second membrane receptor different from said first membrane receptor, preferably said additional sequence corresponding to the C-terminus of said second membrane receptor, and/or
   a substitution of a number of amino acids ranging from 1 to the total number of amino acids of the region delimited by the cytoplasmic tail, preferably a number of amino acids ranging from 1 to 20 amino acids, preferably a number of amino acids ranging from 1 to 15 amino acids, more preferably a number of amino acids ranging from 1 to 10 amino acids with a substitute sequence of a number of amino acids ranging from 1 to the total number of amino acids of the region delimited by the cytoplasmic tail, preferably a number of amino acids ranging from 1 to 20 amino acids, preferably a number of amino acids ranging from 1 to 15 amino acids, more preferably a number of amino acids ranging from 1 to 10 amino acids from a second membrane receptor different from said first membrane receptor,
said hybrid protein being such that said ion channel retains the property of electrical current generation of said ion channel in its natural configuration, and that said first membrane receptor retains the ability to interact with the ligand of said first membrane receptor in its natural configuration,
   as a biosensor for:
the screening of drugs modulating the activity of said first membrane receptor in its natural configuration, and/or
for the in vitro diagnosis of pathologies associated with the presence or the variation of amount of a molecule modifying the activity of said first membrane receptor in its natural configuration.

In one advantageous embodiment the invention relates to the use of a hybrid protein comprising or consisting in
a. the sequence of a first membrane receptor, said first membrane receptor belonging to the G-protein coupled receptors (GPCR) class A family, covalently fused at its C-terminus to
b. the N-terminus sequence of an ion channel, said ion channel belonging to the potassium channel families selected from the inwardly rectifying potassium channels (Kir) family and the voltage-dependent potassium channels (Kᵥ) family,
said ion channel sequence being deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel,
said ion channel possibly containing a tag sequence, said first membrane receptor being liable to present in its cytoplasmic tail at least one mutation, said cytoplasmic tail being a sequence delimited by the first amino acid after the last amino acid of the transmembrane helix and the last amino acid of said first membrane receptor, said mutations consisting in:
a deletion of a number of amino acids ranging from 1 to the total number of amino acids of the region delimited by the cytoplasmic tail, and/or
an addition, of an additional sequence of a number of amino acids ranging from 1 to the total number of amino acids of the region delimited by the cytoplasmic tail, originating from a second membrane receptor different from said first membrane receptor, preferably said additional sequence corresponding to the C-terminus of said second membrane receptor and/or
a substitution of a number of amino acids ranging from 1 to the total number of amino acids of the region delimited by the cytoplasmic tail, with an substitute sequence of a number of amino acids ranging from 1 to the total number of amino acids of the region delimited by the cytoplasmic tail from a second membrane receptor different from said first membrane receptor
said hybrid protein being such that said ion channel retains the property of electrical current generation of said ion channel in its natural configuration, and that said first membrane receptor retains the ability to interact with the ligand of said first membrane receptor in its natural configuration,
   as a biosensor for:
the screening of drugs modulating the activity of said first membrane receptor in its natural configuration, and/or
for the in vitro diagnosis of pathologies associated with the presence or the variation of amount of a molecule modifying the activity of said first membrane receptor in its natural configuration.

In one advantageous embodiment the invention relates to the use of a hybrid protein comprising or consisting in
a. the sequence of a first membrane receptor, said first membrane receptor belonging to the G-protein coupled receptors (GPCR) class A family, covalently fused at its C-terminus to
b. the N-terminus sequence of an ion channel, said ion channel belonging to the potassium channel families selected from the inwardly rectifying potassium channels (Kir) family and the voltage-dependent potassium channels (Kᵥ) family,
c. and possibly containing a linker sequence between the C-terminus of said first membrane receptor and the N-terminus part of said ion channel, said linker being absent in the natural configuration of said first membrane receptor and said ion channel,
said ion channel sequence being deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably being deleted from 1 to 49 amino acids at the N-terminus part of said Kir ion channel, or being deleted from 1 to 435 amino acids at the N-terminus of said Kv ion channel
said ion channel possibly containing a tag sequence,
said first membrane receptor being liable to present in its extracellular domain a mutation allowing the specific interaction with a ligand different from the ligand that interacts with the first membrane receptor in its natural configuration,
said first membrane receptor being liable to present in its cytoplasmic tail, said cytoplasmic tail being a sequence delimited by the first amino acid after the last amino acid of the transmembrane helix and the last amino acid of said first membrane receptor, in particular being liable to present in the 100 amino acids in its C-terminus part
   a deletion of a number of amino acids ranging from 1 to the total number of amino acids of the region delimited by the cytoplasmic tail, preferably of a number of amino acids ranging from 1 to 100, preferably of a number of amino acids ranging from 1 to 70, preferably of a number of amino acids ranging from 1 to 20 preferably of a number of amino acids ranging from 1 to 15, more preferably of a number of amino acids ranging from 1 to 10, amino acids at the C-terminus, provided that said deletion does not affect the transmembrane amino acid sequence of said membrane receptor and/or
   an addition, in particular after the last amino acid at the C-terminus of said first membrane receptor, of an additional sequence of a number of amino acids ranging from 1 to the total number of amino acids of the region delimited by the cytoplasmic tail, preferably of a number of amino acids ranging from 1 to 100 amino acids, preferably of a number of amino acids ranging from 1 to 70, preferably of a number of amino acids ranging from 1 to 20 amino acids, preferably of a number of amino acids ranging from 1 to 15 amino acids, more preferably of a number of amino acids ranging from 1 to 10 amino acids, preferably contiguous, originating from a second membrane receptor different from said first membrane receptor, preferably said additional sequence corresponding to the C-terminus of said second membrane receptor, and/or
   a substitution of a number of amino acids ranging from 1 to the total number of amino acids of the region delimited by the cytoplasmic tail, preferably a number of amino acids ranging from 1 to 20 amino acids, preferably a number of amino acids ranging from 1 to 15 amino acids, more preferably a number of amino acids ranging from 1 to 10 amino acids with an substitute sequence of a number of amino acids ranging from 1 to the total number of amino acids of the region delimited by the cytoplasmic tail, preferably a number of amino acids ranging from 1 to 20 amino acids, preferably a number of amino acids ranging from 1 to 15 amino acids, more preferably a number of amino acids ranging from 1 to 10 amino acids from a second membrane receptor different from said first membrane receptor
said hybrid protein being such that said ion channel retains the property of electrical current generation of said ion channel in its natural configuration, and that said first membrane receptor retains the ability to interact with the ligand of said first membrane receptor in its natural configuration,
   as a biosensor for:
the screening of drugs modulating the activity of said first membrane receptor in its natural configuration, and/or
for the in vitro diagnosis of pathologies associated with the presence or the variation of amount of a molecule modifying the activity of said first membrane receptor in its natural configuration.

In one particular embodiment, the inventions relates to the use of a hybrid protein comprising the sequence of a first membrane receptor fused at its C-terminus to the N-terminus of a ion channel, and possibly containing a linker between the C-terminus of said first membrane receptor and the N-terminus part of said ion channel, said linker being absent in the natural configuration of said first membrane receptor and said ion channel,
said ion channel sequence being deleted of a number of amino acids ranging from 1 to 29 amino acids at the N-terminus part of said ion channel, possibly containing a tag sequence,
said first membrane receptor being liable to present in the 70 amino acids in its C-terminus part
   a deletion of a number of amino acids ranging from 1 to 20, preferably of a number of amino acids ranging from 1 to 15, more preferably of a number of amino acids ranging from 1 to 10, amino acids at the C-terminus, and/or
   an addition, in particular after the last amino acid at the C-terminus of said first membrane receptor, of an additional sequence of a number of amino acids ranging from 1 to 20 amino acids, preferably of a number of amino acids ranging from 1 to 15 amino acids, more preferably of a number of amino acids ranging from 1 to 10 amino acids, preferably contiguous, originating from a second membrane receptor different from said first membrane receptor, preferably said additional sequence corresponding to the C-terminus of said second membrane receptor, and/or
   a substitution of a number of amino acids ranging from 1 to 20 amino acids, preferably a number of amino acids ranging from 1 to 15 amino acids, more preferably a number of amino acids ranging from 1 to 10 amino acids with an substitute sequence of a number of amino acids ranging from 1 to 20 amino acids, preferably a number of amino acids ranging from 1 to 15 amino acids, more preferably a number of amino acids ranging from 1 to 10 amino acids from a second membrane receptor different from said first membrane receptor,
said hybrid protein being such that said ion channel retains the property of electrical current generation of said ion channel in its natural configuration, and that said first membrane receptor retains the ability to interact with the ligand of said first membrane receptor in its natural configuration,
   as a biosensor for:
the screening of drugs modulating the activity of said first membrane receptor in its natural configuration, and/or
for the in vitro diagnosis of pathologies associated with the presence or the variation of amount of a molecule modifying the activity of said first membrane receptor in its natural configuration.

The invention is based on the unexpected observation made by the inventors that a hybrid protein comprising or constituted by a membrane receptor fused to an ion channel, modified in its N-terminus part, can be used as efficient biosensor to measure the membrane receptor activity. This observation is based on the fact that the ion channel and the membrane receptor, involved in the hybrid protein, retain the biological function of the corresponding proteins in their natural configuration.

Then, to summarize, the hybrid proteins disclosed in the invention can be constituted as follows:

1—a membrane receptor sequence, in its natural configuration, fused to the N-terminus deleted sequence of a ion channel, or
2—a membrane receptor sequence, deleted in its C-terminus part, fused to the N-terminus deleted sequence of a ion channel, or
3—a membrane receptor sequence, having an addition at the C-terminus of an additional sequence from a second membrane receptor, fused to the N-terminus deleted sequence of a ion channel, or
4—a membrane receptor sequence, having a substitution at the C-terminus with a substitution sequence from a second membrane receptor, fused to the N-terminus deleted sequence of a ion channel.

Possibly, a linker can be present between the sequence of the membrane receptor and the sequence of the ion channel.

Thus, the hybrid proteins disclosed in the invention can be constituted as follows:

1—a membrane receptor sequence, in its natural configuration, fused to a linker sequence, said linker sequence being fused to the N-terminus deleted sequence of a ion channel, or
2—a membrane receptor sequence, deleted in its C-terminus part, fused to a linker sequence, said linker sequence being fused to the N-terminus deleted sequence of a ion channel, or
3—a membrane receptor sequence, having an addition at the C-terminus of an additional sequence from a second membrane receptor, fused to a linker sequence, said linker sequence being fused to the N-terminus deleted sequence of a ion channel, or
4—a membrane receptor sequence, having an substitution at the C-terminus with a substitution sequence from a second membrane receptor, fused to the N-terminus deleted sequence of a ion channel, fused to a linker sequence, said linker sequence being fused to the N-terminus deleted sequence of a ion channel.

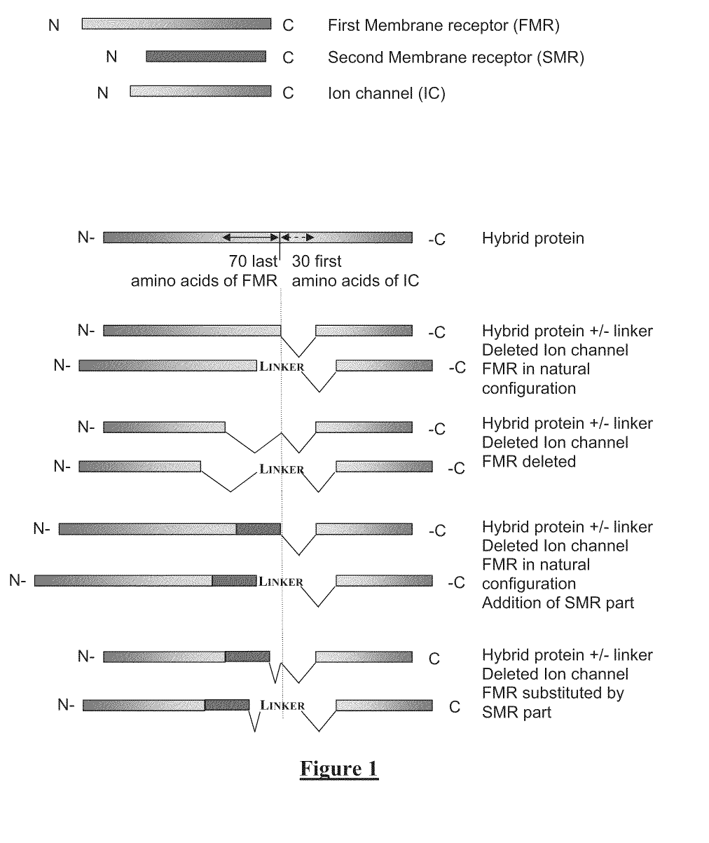

These hybrid proteins are illustrated in the FIG. 1.

The term “and/or” wherever used herein includes the meaning of “and”, “or” and “all or any other combination of the elements connected by said term”.

The terms “after” and “before” refer respectively to the position of an amino acid immediately following or preceding the position of an other amino acid, in the same amino acid sequence, starting at the N-terminal and ending at the C-terminal

The terms “first” and “last” refer respectively to the position of an amino acid located at the first position or at the end of an amino acid sequence starting at the N-terminal and ending at the C-terminal.

In the invention, the hybrid protein is constituted by at least a first protein fused in its C-terminus part to the N-terminus of a second protein. Possibly, the hybrid protein according to the invention can be constituted by the fusion of a first C-terminus part to the N-terminus of a second protein

In the invention, the term “linker” refers to amino acid sequence linking the first part of the hybrid protein to the second part. In particular, linker, according to the invention, is an amino acid sequence comprising or consisting of at least one amino acid. This linking sequence is absent in the natural sequence of said first or said second membrane receptor and in the natural sequence of said ion channel, i.e., linker is absent in the natural configuration of said first membrane receptor and said ion channel.

According to the invention, the N-terminus part and C-terminus part respectively correspond to the first half part of a protein and to the second half part. For example, for a protein containing 10 amino acids, the 5 first amino acids correspond to the N-terminus and the 5 last amino acids correspond to C-terminus. This definition is a broad definition of N-terminus part and C-terminus part commonly accepted in the art.

Therefore, terms “sequence delimited by the first amino acid after the last transmembrane helix and the last amino acid of said membrane receptor” means that the sequence begins at the position corresponding to the first amino acid following the last amino acid of the transmembrane helix and extends to the end of the protein.

According to the invention, “a hybrid protein” defines a fusion protein. A fusion protein, also known as a chimeric protein, is a protein created through the joining of two or more genes which originally coded for separate proteins. The translation of this fusion gene results in a single polypeptide with functional properties derived from each of the original proteins. Recombinant fusions proteins are created artificially by routine protocols known in the art for their use in biological research or therapeutics.

### Receptors

According to the invention, a “membrane receptor” is a protein on the cell membrane that binds to a specific molecule (a ligand), and initiates the cellular response to the ligand. Ligand-induced changes in the behavior of receptor proteins result in physiological changes that constitute the biological actions of the ligand.

According to the invention, the membrane receptor proteins can exist in 2 forms such as peripheral membrane proteins and transmembrane proteins. Preferably, the membrane receptors of the invention are transmembrane receptors. These receptors are embedded in the phospholipid bilayer of cell membranes and allow the activation of signal transduction pathways in response to the activation by the binding molecule.

Transmembrane receptor contains 3 conserved domains: an extracellular domain that allows the interaction with the ligand, a transmembrane domain that includes at least 1 hydrophobous α-helix, and an intracellular domain which serves as an anchor platform for adaptator molecules.

According to the invention, the “cytoplasmic tail” corresponds to the cytoplasmic domain. It is structurally defined by the amino acid sequence delimited by the first amino acid after the transmembrane domain (after the hydrophobous α-helix) to the last amino acid of the receptor. It is common for a person with a skill to easily define without ambiguity the transmembrane α-helix of a membrane protein. Therefore, the terms “region delimited by the cytoplasmic tail” means “cytoplasmic tail” or cytoplasmic domain.

The terms “first membrane receptor” refer to the above definition of membrane receptor.

As ligand, it is defined according to the invention, any molecule able to specifically interact with said receptor, and optionally able to modify receptor activity or function. For example, said ligand can reduce or enhance signal generated in cell after said receptor activation.

These above-mentioned molecules can be the natural ligand of said receptor, i.e., the molecule that naturally interacts with said receptor, or any other molecules such as antibodies, artificial, chemical or biological compound liable to form a stable interaction with said receptor.

According to the invention, “last amino acids after the transmembrane domain” means the amino acids present in the sequence of amino acids that immediately follows the sequence of the transmembrane domain of said first membrane receptor. “amino acid that immediately follows the sequence” can be the first amino acid immediately after the transmembrane domain, or the second amino acid, or the third amino acid.

According to the invention, “sequence delimited by the last amino acids after the transmembrane domain to the last amino acid of said first membrane receptor” defines a sequence that begins at the first amino acid that immediately follows the sequence of transmembrane domain and extends to the last amino acid of the first membrane receptor, at the C-terminus part.

In one advantageous embodiment, the extracellular domain of the first membrane receptor can be mutated, in particular in the domain that allows the interaction with the ligand. This mutation (substitution, deletion, insertion) does not modify the tri-dimensional conformation neither of the intracellular domain, nor of the complete protein, but allows the mutated receptor to interact with a ligand different from the ligand that interacts with the first membrane receptor in its natural configuration.

Indeed, it has been recently demonstrated that mutations in the extracellular domain, preferably in the ligand interaction domain, can transform the receptor ligand specificity to allow the interaction with any ligand, as defined above [Armbruster B N, Li X Pausch M H, Herlitze S, Roth B L (2007) Evolving the lock to fit the key to create a family of G protein-coupled receptors potently activated by an inert ligand. Proc Natl Acad Sci USA. 104:5163-8].

In a preferred embodiment, the membrane receptor according to the invention belongs to the GPCR class A membrane receptor. Thus, de facto, natural membrane receptor containing ion channel sequence, such as nicotinic receptor, or 5HT3 receptor are excluded.

### Ion Channels

According to the invention, ion channel is defined as a pore-forming protein that helps to establish, and control, the voltage gradient across the plasma membrane of all living cells (cell membrane potential) [Hille B (2001) Ionic channels of excitable membranes, 3^{rd} edition, Sinauer Associates, Sunderland, M A] Ion channels allow the flow of ions down their electrochemical gradient. They are present in the membranes that surround all biological cells.

Ion channels regulate the flow of ions across the membrane in all cells. They are integral membrane proteins; or, more typically, assemblies of several proteins. Such “multi-subunit” assemblies usually involve a circular arrangement of identical or homologous proteins closely packed around a water-filled pore through the plane of the membrane or lipid bilayer. The pore-forming subunits are often called a subunits, while the auxiliary subunits are denoted β, γ, and so on. While some channels permit the passage of ions based solely on charge, the archetypal channel pore is just a few atoms wide at its narrowest point. It conducts a specific species of ion, such as sodium or potassium, and conveys them through the membrane single file—nearly as quickly as the ions move through free fluid. In some ion channels, passage through the pore is governed by a “gate,” which may be opened or closed by chemical or electrical signals, temperature, or mechanical force, depending on the variety of channel [Hille B (2001) Ionic channels of excitable membranes, 3^{rd} edition, Sinauer Associates, Sunderland, M A]

The advantageous ions channels of the invention are the potassium channel families selected from
the inwardly rectifying potassium channels (Kir) family and
the voltage-dependent potassium channels (Kᵥ) family.

In the invention, a channel that is “inwardly-rectifying” is one that passes current (positive charge) more easily in the inward direction (into the cell). It is thought that this current may play an important role in regulating the resting level of neuronal activity.

To date, 15 members of the Kir family have been identified: Kir1.1, Kir2.1, Kir2.2, Kir2.3, Kir2.4, Kir3.1, Kir3.2, Kir3.3, Kir3.4, Kir4.1, Kir4.2, Kir5.1, Kir6.1, Kir6.2 and Kir7.1.

In the invention “voltage-dependant potassium channels” are transmembrane channels specific for potassium and sensitive to voltage changes in the cell's membrane potential. They play a crucial role during action potentials in returning the depolarized cell to a resting state. To date, 40 voltage-dependant potassium channels alpha subunits alpha are known in human, the alpha subunit of Kv ion channels form the actual conductance pore.

The more preferred Kv receptors according to the invention are Kv1.1, Kv1.2, Kv1.3, Kv1.4, Kv1.5, Kv1.6, Kv1.7, Kv1.8, Kv2.1, Kv2.2, Kv3.1, Kv3.2, Kv3.3, Kv3.4, Kv4.1, Kv4.2, Kv4.3, Kv5.1, Kv6.1, Kv6.2, Kv6.3, Kv6.4, Kv7.1, Kv7.2, Kv7.3, Kv7.4, Kv7.5, Kv8.1, Kv8.2, Kv9.1, Kv9.2, Kv9.3, Kv10.1, Kv10.2, Kv11.1, Kv11.2, Kv11.3, Kv12.1, Kv12.2 and Kv12 (see for instance [Gutman et al. 2005, Pharmacol Rev 57:473-508])

There are strong sequence homologies among all channels of the Kir family (Kir1.1 to Kir7.1). This suggests a common architecture that would be similar to that of Kir channels from bacteria [Kuo et al. (2003) Science. 300:1922-6].

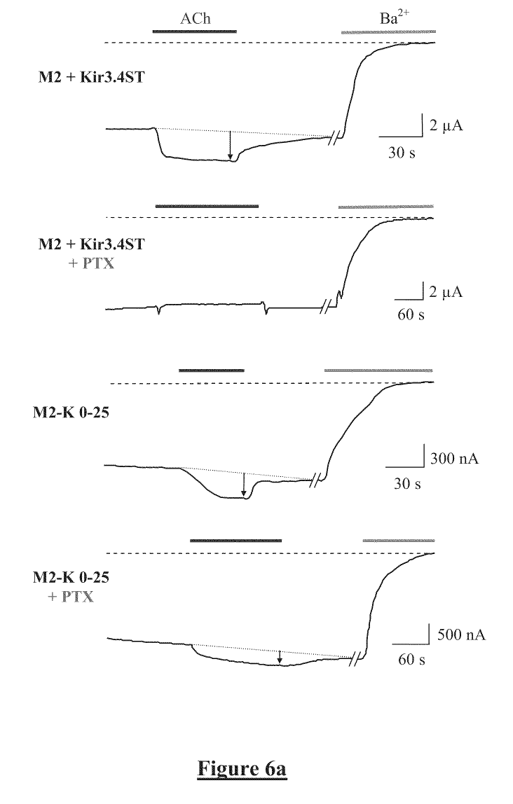

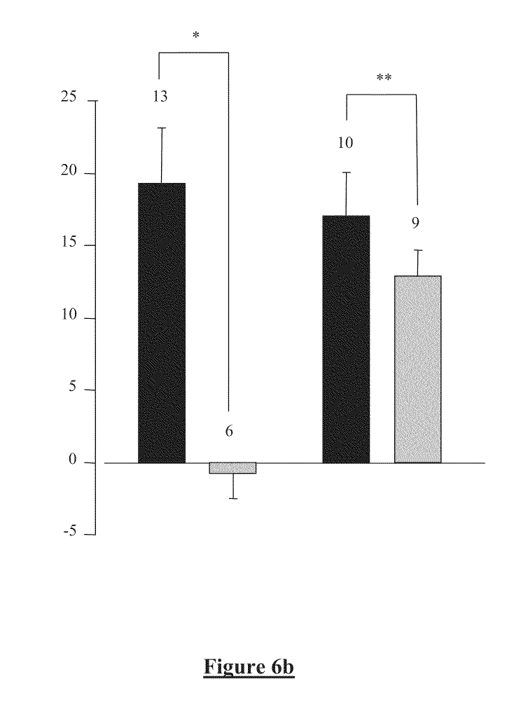

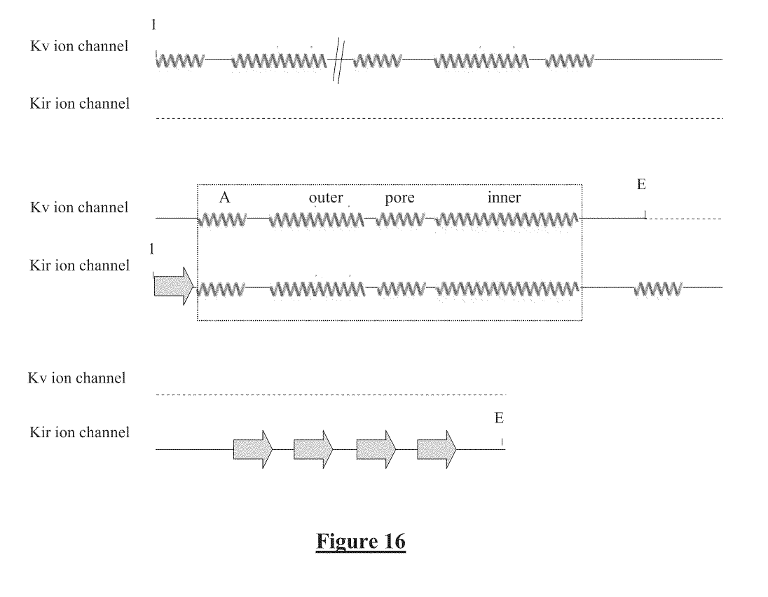

Moreover, comparing the structures of tetrameric voltage-dependent K channels [Long et al, (2005) Science. 309:897-903] and of Kir channels [Kuo et al. (2003) Science. 300:1922-6] demonstrates that the core regions (slide helix+ last two transmembrane helices) are identical in structure despite divergence in sequences, and that voltage-dependent channels are much like a voltage-sensing domain attached to a Kir channel [FIG. 1 of Swartz et al., (2008) Nature. 456:891-7; FIG. 6c&d of Long et al., (2007) Nature. 450:376-82]. The common structure between these two types of ions channels is represented in the grey box in FIG. 16.

Ion channels differ between each other by the ion they let pass (for example, Na⁺, K⁺, Cl⁻, Ca²⁺), their regulation, the number of subunits which compose the pore and other structural aspects. Channels belonging to the largest class of the voltage-gated channels consist of four subunits, each subunit having six transmembrane helices. Upon activation, these helices move about and open the pore. Two of these six helices are separated by a loop that lines the pore and is the primary determinant of ion selectivity and conductance in this channel class and some others.

The channel subunits of one such other class, for instance, consist of “Pore” loop and have two transmembrane helices.

The two helices, found in all ions channels are easy to identify, from the amino acid sequence, to a person with ordinary skill.

According to the invention, terms “deleted of a number of amino acids ranging from 1 to the total number of amino acids of the region” means that one sequence of amino acids can be deleted of 1, or 2, or 3, etc. . . . . to all the amino acids that constitute said sequence of amino acids. For instance, if a sequence of amino acids consists of 50 amino acids, the sequence can be deleted of 1, or 2, or 3, or 4 . . . or 48, or 49 or 50 amino acids. The deletion of 50 amino acids then consists of a deletion of all amino acids.

According to the invention, the phrase “deleted from the first amino acid at the N-terminus part of said ion channel to the first amino acid of the cytoplasmic α-helix that precedes the first of the two transmembrane α-helices that form the pore region of said potassium channel” means that all amino acids from the first amino acid of the channel, e.g., from the Methionine, to the first amino acid of the cytoplasmic α-helix that precedes the first of the two transmembrane α-helices that form the pore region of said potassium channel can be deleted.

According to the invention, “first amino acid of the cytoplasmic α-helix that precedes the first of the two transmembrane α-helices that form the pore region of said potassium channel” defined a region at the N-terminus of the α-helix region, comprising 1 to 5 amino acids, that are characteristic of the α-helix domain, but that can be deleted, without modification of the α-helix structure, and without modifying the pore structure. 1 to 5 amino acids means 1, or 2 or 3 or 4 or 5 amino acids.

According to the invention, the hybrid protein is constituted by the fusion of two different proteins. Then, even if the two proteins involved have a different size in their amino acid sequences, it is considered that the first protein, i.e. membrane receptor, corresponds to the N-terminus of the hybrid protein, and the second protein, i.e. ion channel, corresponds to the C-terminus of the hybrid protein.

Terms “N-terminus”, “amino terminal”, “amino terminus” and “N-ter” are uniformly used hereafter to designate the first part of a protein. Terms “C-terminus”, “carboxy terminal”, “carboxy terminus” and “C-ter” are uniformly used hereafter to designate the second part of a protein.

In summary, the hybrid protein of the invention comprises, in its amino terminal part, the sequence of a membrane receptor, optionally truncated and optionally fused to a linker, i.e. a linking sequence, said linker itself being linked to the truncated sequence of a ion channel, said truncated sequence of ion channel corresponding to the carboxy-terminal part of the hybrid protein.

Regarding the deletion at the N-terminus of the Kir ion channel, it is disclosed in the Example section hereafter that deletion of 25 amino acids of the N-terminus part of Kir6.2 allows the generation of an electric flux when said deleted Kir6.2 is fused to a membrane receptor.

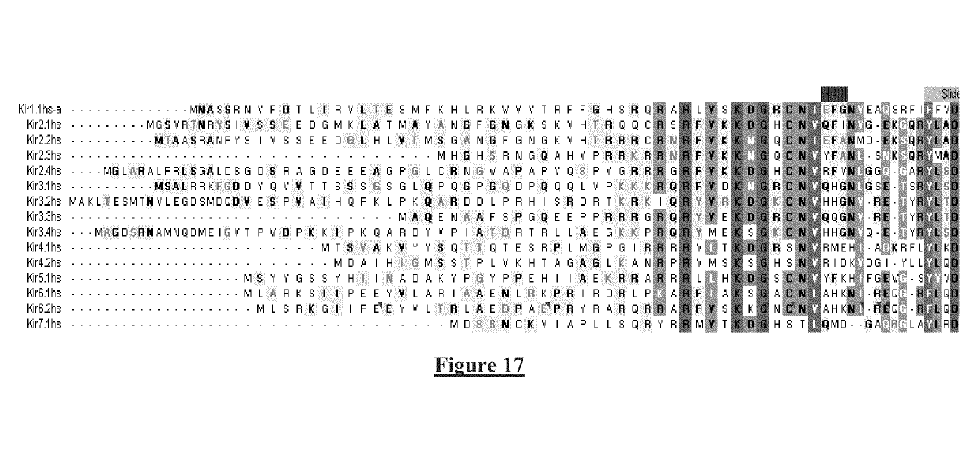

Based on this observation, a skilled person knows that such a deletion can be carried out in the other Kir ion channel. By aligning sequences of Kir channel, as shown in FIG. 17, the skilled person knows that he must delete the N-terminus part of the Kir channels in order to obtain a similar deletion with those obtained in Kir6.2.

In other words, the skilled person can easily determine the exact number of amino acid he have to delete at the N-terminus of another Kir ion channel, by comparing its sequence to Kir6.2 sequence.

In other words, a deletion of the Kir channel from the first amino acid at the N-terminus part of said Kir channel to the first amino acid of the cytoplasmic α-helix that precedes the first of the two transmembrane α-helices that form the pore region of said potassium channel means preferably that:

the sequence of kir1.1 is deleted of one amino acid to the total number of amino acids extending from the first amino acid to the amino acid at the position 36, the sequence of kir2.1 is deleted of one amino acid to the total number of amino acids extending from the first amino acid to the amino acid at the position 41, the sequence of kir2.2 is deleted of one amino acid to the total number of amino acids extending from the first amino acid to the amino acid at the position 40, the sequence of kir2.3 is deleted of one amino acid to the total number of amino acids extending from the first amino acid to the amino acid at the position 15, the sequence of kir2.4 is deleted of one amino acid to the total number of amino acids extending from the first amino acid to the amino acid at the position 46, the sequence of kir3.1 is deleted of one amino acid to the total number of amino acids extending from the first amino acid to the amino acid at the position 40, the sequence of kir3.2 is deleted of one amino acid to the total number of amino acids extending from the first amino acid to the amino acid at the position 51, the sequence of kir3.3 is deleted of one amino acid to the total number of amino acids extending from the first amino acid to the amino acid at the position 18, the sequence of kir3.4 is deleted of one amino acid to the total number of amino acids extending from the first amino acid to the amino acid at the position 47, the sequence of kir4.1 is deleted of one amino acid to the total number of amino acids extending from the first amino acid to the amino acid at the position 24, the sequence of kir4.2 is deleted of one amino acid to the total number of amino acids extending from the first amino acid to the amino acid at the position 23, the sequence of kir5.1 is deleted of one amino acid to the total number of amino acids extending from the first amino acid to the amino acid at the position 30, the sequence of kir6.1 is deleted of one amino acid to the total number of amino acids extending from the first amino acid to the amino acid at the position 30, the sequence of kir6.2 is deleted of one amino acid to the total number of amino acids extending from the first amino acid to the amino acid at the position 29 and the sequence of kir7.1 is deleted of one amino acid to the total number of amino acids extending from the first amino acid to the amino acid at the position 14.

The terms “deleted of one amino acid to the total number of amino acids extending from the first amino acid to the amino acid at the position 36” means that the corresponding Kir1.1 sequence is deleted of the first amino acid, or of the first and the second amino acid, or of the first and the second and the third amino acid, or of the first and the second and the third and the fourth amino acid . . . or from the first to the thirty sixth amino acid. This definition applies mutatis mutandis to all above cited Kir receptors.

In an advantageous embodiment, the hybrid fusion according to the invention comprises a Kir ion channel such as:

the sequence of kir1.1 is deleted of one amino acid to the total number of amino acids extending from the first amino acid to the amino acid at the position 32, the sequence of kir2.1 is deleted of one amino acid to the total number of amino acids extending from the first amino acid to the amino acid at the position 37, the sequence of kir2.2 is deleted of one amino acid to the total number of amino acids extending from the first amino acid to the amino acid at the position 36, the sequence of kir2.3 is deleted of one amino acid to the total number of amino acids extending from the first amino acid to the amino acid at the position 11, the sequence of kir2.4 is deleted of one amino acid to the total number of amino acids extending from the first amino acid to the amino acid at the position 42, the sequence of kir3.1 is deleted of one amino acid to the total number of amino acids extending from the first amino acid to the amino acid at the position 36, the sequence of kir3.2 is deleted of one amino acid to the total number of amino acids extending from the first amino acid to the amino acid at the position 46, the sequence of kir3.3 is deleted of one amino acid to the total number of amino acids extending from the first amino acid to the amino acid at the position 14, the sequence of kir3.4 is deleted of one amino acid to the total number of amino acids extending from the first amino acid to the amino acid at the position 43, the sequence of kir4.1 is deleted of one amino acid to the total number of amino acids extending from the first amino acid to the amino acid at the position 20, the sequence of kir4.2 is deleted of one amino acid to the total number of amino acids extending from the first amino acid to the amino acid at the position 19, the sequence of kir5.1 is deleted of one amino acid to the total number of amino acids extending from the first amino acid to the amino acid at the position 26, the sequence of kir6.1 is deleted of one amino acid to the total number of amino acids extending from the first amino acid to the amino acid at the position 26, the sequence of kir6.2 is deleted of one amino acid to the total number of amino acids extending from the first amino acid to the amino acid at the position 25 and the sequence of kir7.1 is deleted of one amino acid to the total number of amino acids extending from the first amino acid to the amino acid at the position 10.

In a most advantageous embodiment, the hybrid protein according to the invention comprises a Kir channel such as:

the sequence of kir1.1 is deleted of all its first 32 amino acids at the N-terminus, the sequence of kir2.1 is deleted of all its first 37 amino acids at the N-terminus, the sequence of kir2.2 is deleted of all its first 36 amino acids at the N-terminus, the sequence of kir2.3 is deleted of all its first 11 amino acids at the N-terminus, the sequence of kir2.4 is deleted of all its first 42 amino acids at the N-terminus, the sequence of kir3.1 is deleted of all its first 36 amino acids at the N-terminus, the sequence of kir3.2 is deleted of all its first 46 amino acids at the N-terminus, the sequence of kir3.3 is deleted of all its first 14 amino acids at the N-terminus, the sequence of kir3.4 is deleted of all its first 43 amino acids at the N-terminus, the sequence of kir4.1 is deleted of all its first 20 amino acids at the N-terminus, the sequence of kir4.2 is deleted of all its first 19 amino acids at the N-terminus, the sequence of kir5.1 is deleted of all its first 26 amino acids at the N-terminus, the sequence of kir6.1 is deleted of all its first 26 amino acids at the N-terminus, the sequence of kir6.2 is deleted of all its first 25 amino acids at the N-terminus and the sequence of kir7.1 is deleted of all its first 10 amino acids at the N-terminus.

According to the invention, in preferred hybrid proteins described, the Kv ion channel amino acid sequence is preferably deleted “of a number of amino acids ranging from 1 to 435 amino acids at the N-terminus part of said Kv ion channel”. These terms mean that the Kv ion channel sequence is deleted of 1, or 2, or 3, or 4, or 5, or 6, or 7, or 8, or 9, or 10, or 11, or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or 40, or 41, or 42, or 43, or 44, or 45, or 46, or 47, or 48, or 49, or 50, or 51, or 52, or 53, or 54, or 55, or 56, or 57, or 58, or 59, or 60, or 61, or 62, or 63, or 64, or 65, or 66, or 67, or 68, or 69, or 70, or 71, or 72, or 73, or 74, or 75, or 76, or 77, or 78, or 79, or 80, or 81, or 82, or 83, or 84, or 85, or 86, or 87, or 88, or 89, or 90, or 91, or 92, or 93, or 94, or 95, or 96, or 97, or 98, or 99, or 100, or 101, or 102, or 103, or 104, or 105, or 106, or 107, or 108, or 109, or 110, or 111, or 112, or 113, or 114, or 115, or 116, or 117, or 118, or 119, or 120, or 121, or 122, or 123, or 124, or 125, or 126, or 127, or 128, or 129, or 130, or 131, or 132, or 133, or 134, or 135, or 136, or 137, or 138, or 139, or 140, or 141, or 142, or 143, or 144, or 145, or 146, or 147, or 148, or 149, or 150, or 151, or 152, or 153, or 154, or 155, or 156, or 157, or 158, or 159, or 160, or 161, or 162, or 163, or 164, or 165, or 166, or 167, or 168, or 169, or 170, or 171, or 172, or 173, or 174, or 175, or 176, or 177, or 178, or 179, or 180, or 181, or 182, or 183, or 184, or 185, or 186, or 187, or 188, or 189, or 190, or 191, or 192, or 193, or 194, or 195, or 196, or 197, or 198, or 199, or 200, or 201, or 202, or 203, or 204, or 205, or 206, or 207, or 208, or 209, or 210, or 211, or 212, or 213, or 214, or 215, or 216, or 217, or 218, or 219, or 220, or 221, or 222, or 223, or 224, or 225, or 226, or 227, or 228, or 229, or 230, or 231, or 232, or 233, or 234, or 235, or 236, or 237, or 238, or 239, or 240, or 241, or 242, or 243, or 244, or 245, or 246, or 247, or 248, or 249, or 250, or 251, or 252, or 253, or 254, or 255, or 256, or 257, or 258, or 259, or 260, or 261, or 262, or 263, or 264, or 265, or 266, or 267, or 268, or 269, or 270, or 271, or 272, or 273, or 274, or 275, or 276, or 277, or 278, or 279, or 280, or 281, or 282, or 283, or 284, or 285, or 286, or 287, or 288, or 289, or 290, or 291, or 292, or 293, or 294, or 295, or 296, or 297, or 298, or 299, or 300, or 301, or 302, or 303, or 304, or 305, or 306, or 307, or 308, or 309, or 310, or 311, or 312, or 313, or 314, or 315, or 316, or 317, or 318, or 319, or 320, or 321, or 322, or 323, or 324, or 325, or 326, or 327, or 328, or 329, or 330, or 331, or 332, or 333, or 334, or 335, or 336, or 337, or 338, or 339, or 340, or 341, or 342, or 343, or 344, or 345, or 346, or 347, or 348, or 349, or 350, or 351, or 352, or 353, or 354, or 355, or 356, or 357, or 358, or 359, or 360, or 361, or 362, or 363, or 364, or 365, or 366, or 367, or 368, or 369, or 370, or 371, or 372, or 373, or 374, or 375, or 376, or 377, or 378, or 379, or 380, or 381, or 382, or 383, or 384, or 385, or 386, or 387, or 388, or 389, or 390, or 391, or 392, or 393, or 394, or 395, or 396, or 397, or 398, or 399, or 400, or 401, or 402, or 403, or 404, or 405, or 406, or 407, or 408, or 409, or 410, or 411, or 412, or 413, or 414, or 415, or 416, or 417, or 418, or 419, or 420, or 421, or 422, or 423, or 424, or 425, or 426, or 427, or 428, or 429, or 430, or 431, or 432, or 433, or 434, or 435 amino acids. As mentioned above, the skilled person can easily, by comparing Kir and Kv ion channels sequences by using software (clustal . . . ), determine the exact number of amino acids he has to delete at the N-terminus of a determined Kv receptor.

The invention is based on the observation that the deletion of the N-terminus part of ion channel confers to the hybrid protein a highly unexpected sensitivity in current generation. Indeed, whereas a hybrid protein comprising membrane receptor fused to non-truncated (at the N-terminus part) ion channel is not able to generate an efficiently detectable electrical signal when a molecule binds to the receptor, the hybrid protein of the invention is able to generate a highly detectable electrical signal.

Then, all the hybrid proteins described hereafter are therefore deleted at the N-terminus part of the ion channel sequence, as above-mentioned.

In a preferred embodiment, the deletion of amino acids in the first part of said ion channel is a deletion of contiguous amino acids.

As tag, it is defined in the invention, a peptide, a polypeptide originating from a protein that differs from the protein of interest, i.e. membrane receptor or ion channel. Tags allow the purification of protein containing them, and are used because some specific antibodies directed against these sequences are available.

Tag sequence is inserted in the hybrid protein sequence either at the C-terminus or the N-terminus part of the hybrid protein, i.e. at the N-terminus part of the membrane receptor part of said hybrid protein or at the C-terminus part of the ion channel part of said hybrid protein.

The tag sequence can also be inserted into the sequence of hybrid protein, with the proviso that this insertion does not modify the membrane receptor and ion channel properties.

The hybrid protein defined in the invention, with a deleted sequence in the ion channel part, possibly contains in the membrane receptor part a modification of the C-terminus part, particularly in the 100 last amino acids. This modification, also called mutation, can be a deletion, an addition or a substitution.

In the case of a deletion, the amino terminus part of membrane receptor defined above is deleted of 1 or more, to 100 amino acids. Terms “of 1 or more, to 100 amino acids” means that the deletion corresponds to a deletion of 1, or 2, or 3, or 4, or 5, or 6, or 7, or 8, or 9, or 10, or 11, or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or 40, or 41, or 42, or 43, or 44, or 45, or 46, or 47, or 48, or 49, or 50, or 51, or 52, or 53, or 54, or 55, or 56, or 57, or 58, or 59, or 60, or 61, or 62, or 63, or 64, or 65, or 66, or 67, or 68, or 69, or 70, or 71, or 72, or 73, or 74, or 75, or 76, or 77, or 78, or 79, or 80, or 81, or 82, or 83, or 84, or 85, or 86, or 87, or 88, or 89, or 90, or 91, or 92, or 93, or 94, or 95, or 96, or 97, or 98, or 99, or 100 amino acids. The deletion preferably begins at the last amino acid in the C-terminus membrane receptor sequence and extend from 1 to 100 amino acids, and extends until to 100 amino acids in the direction of the N-terminus of said membrane receptor. In particular embodiment, the deletion mentioned above covers a deletion from 1 to 70, preferably from 1 to 20, more preferably from 1 to 15 amino acids in the C-terminus part of membrane receptors as defined above. In another particular embodiment, the deletion extends from 1 to 10 amino acids as defined above.

The truncation defined above is preferably contiguous, which means that, if two amino acids are deleted, the deletion concerns two contiguous amino acids. The same rule is applied for more than two amino acids.

In the case of addition, one or more amino acids of a second membrane receptor are added to the sequence of said first membrane receptor. The terms “second membrane receptor” refer to the above definition of membrane receptor. The second membrane receptor is different from the first receptor, which means that said second membrane receptor has an amino acid sequence that differs from the amino acid sequence of said first membrane receptor. Said first and said second membrane receptors can belong to the same family of membrane receptors. For example, for illustrating the concept, the first membrane receptor can be the erythropoietin receptor and the second membrane receptor can be the thrombopoietin receptors. These two receptors belong to the cytokine receptor family, but do not have the same amino acid sequence.

The terms “an additional sequence of 1 to 100 amino acids,” means that 1, or 2, or 3, or 4, or 5, or 6, or 7, or 8, or 9, or 10, or 11, or 12, or 13, or 14, or 15, or 16, or 17, or 18, or 19, or 20, or 21, or 22, or 23, or 24, or 25, or 26, or 27, or 28, or 29, or 30, or 31, or 32, or 33, or 34, or 35, or 36, or 37, or 38, or 39, or 40, or 41, or 42, or 43, or 44, or 45, or 46, or 47, or 48, or 49, or 50, or 51, or 52, or 53, or 54, or 55, or 56, or 57, or 58, or 59, or 60, or 61, or 62, or 63, or 64, or 65, or 66, or 67, or 68, or 69, or 70, or 71, or 72, or 73, or 74, or 75, or 76, or 77, or 78, or 79, or 80, or 81, or 82, or 83, or 84, or 85, or 86, or 87, or 88, or 89, or 90, or 91, or 92, or 93, or 94, or 95, or 96, or 97, or 98, or 99, or 100 amino acids originating from the second membrane receptor are added to the sequence of the first membrane receptor.

In one preferred embodiment, the amino acids originating from said second membrane receptor are added directly after the last amino acid of the C-terminus part of said first membrane receptor.

In the case of substitution, one or more amino acids of a second membrane receptor replace one or more amino acids of the sequence of said first membrane receptor. The term “substitute sequence” means that the amino acids sequence of said second membrane receptor takes the place of the amino acids of said first membrane receptor.

According to the invention, the substitution concerns 1, or 2, or 3, or 4, or 5, or 6, or 7, or 8, or 9, or 10, or 11, or 12, or 13, or 14, or 15, or 16, or 17, or 18, or 19, or 20 amino acids originating from first membrane receptor that are replaced by 1, or 2, or 3, or 4, or 5, or 6, or 7, or 8, or 9, or 10, or 11, or 12, or 13, or 14, or 15, or 16, or 17, or 18, or 19, or 20 amino acids originating from first membrane receptor. The length of substitute sequence and the length of the substituted sequence are preferably the same. Preferably, the substitution concerns contiguous amino acids, as defined above.

In a particular embodiment, the above defined substitution occurs in the C-terminus part of said first membrane receptor, and the substitution sequence originates from the C-terminus part of said second membrane receptor.

In addition, the above mentioned hybrid proteins, with or without tag sequence, can be mutated in the extracellular domain of the first membrane receptor.

The terms “channel retains the property of electrical signal generation” are defined, according to the invention, by the fact that the ion channel part, contained in the hybrid protein, is able to generate an ionic current with a similar and/or equivalent and/or best efficiency that the ionic current generated by the ion channel naturally existing in non-manipulated cells.

The ionic current generated by an ion channel in its natural configuration or by the hybrid protein is easy to detect with routine protocols and materials of electrophysiology, such as patch clamp, microelectrode recordings, or artificial lipid bilayer recordings [Hamill O P, Marty A, Neher E, Sakmann B, Sigworth F J (1981) Improved patch-clamp techniques for high-resolution current recording from cells and cell-free membrane patches. Pflügers Arch-Eur J. Physiol. 391:85-100; Ashcroft F M (2000) Studying ion channels. In: Ion channels and disease, Academic Press; Priest B T, Swensen A M, McManus O B (2007) Automated electrophysiology in drug discovery. Curr Pharm Des. 13:2325-37].

The terms “membrane receptor retains the ability to interact with the ligand” are defined, according to the invention, by the membrane receptor being able to interact with a ligand, as defined above, with an efficiency similar and/or equivalent to that observed when ligand and membrane receptor naturally exist in non-manipulated cells.

The determination of ligand binding affinity, by measuring binding-dependent signals at different concentrations of ligand, are used to compare the ability of a membrane receptor in its natural configuration and the membrane receptor comprised in the hybrid protein, to interact with a ligand. Established techniques to measure receptor affinity include the use of labelled ligands to quantify binding or the detection of products (GTP, cAMP) resulting from the ligand-induced activation of receptors [Thomsen W, Frazer J, Unett D (2005) Functional assays for screening GPCR targets. Curr Opin Biotechnol. 16:655-65].

By “natural configuration” it is meant in the invention that protein is in a configuration that corresponds to the configuration naturally found in cells, when the protein is normally expressed by the translation of the corresponding gene product.

Also, “in natural configuration” means that protein can be purified and isolated from cells, said isolation, and/or purification, not altering their configuration.

According to the invention, the hybrid protein is used as a biosensor for the screening of drugs modulating the activity. By “drug modulating the activity” it is meant any chemical or biological compound or molecule able to modulate, the receptor activity.

The modulation concerns an increase or a decrease of membrane receptor activity.

According to the invention, the drug modifies the activity of the hybrid protein defined above, said modification in the activity being measured by the variation of the electric current generated by the ion channel part of said hybrid protein. Since the membrane receptor part of said hybrid protein retains the properties of the membrane receptor in its natural configuration, said drug will be able to modulate the normal activity of the membrane receptor in its natural configuration.

In the invention, the hybrid protein can also be used as a biosensor for the in vitro diagnosis of pathologies associated with the presence or the variation of amount of a molecule modifying the activity of said first membrane receptor in its natural configuration.

As defined above, since the membrane receptor part of said hybrid protein retains the properties of the membrane receptor in its natural configuration, any ligand of membrane receptor in its natural configuration will be able to interact with the membrane receptor part of said hybrid protein. Then, if a pathology is associated with the presence, the absence or the variation of amount of a natural ligand of a membrane receptor in its natural configuration, the variation of hybrid protein generated current allows to determine the presence, the absence or the variation of amount of a natural ligand in a biological sample.

In an advantageous embodiment, the invention relates to the use of a hybrid protein defined above, wherein said ion channel sequence is deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably is deleted from 1 to 49 amino acids at the N-terminus part of said Kir ion channel, or is deleted from 1 to 435 amino acids at the N-terminus of said Kv ion channel, the numbering being defined from the first amino acid at the N-terminus of said ion channel in its natural configuration.

According to the invention, the terms “the numbering being defined from the first amino acid at the N-terminus of said ion channel in its natural configuration” means that position 1 correspond to the initial methionine of the ion channel. By extension, position 2 corresponds to the amino acid immediately after the initial methionine . . . .

According to the invention, terms “deleted from the sequence delimited by amino acids in position 1 to amino acid corresponding to the first amino acids of the transmembrane α-helix” means that the deletion corresponds to the N-terminus part of the ion channel that is not inserted in the membrane. When the ion channel amino acid sequence or its structure can predict an hydrophobous α-helix (i.e. a putative transmembrane domain), the deletion must be stopped immediately before this α-helix.

According to the invention, the deletion from the position 1 to the position 29 means that the deletion covers an amino acid sequence of 29 contiguous amino acids. Also, from the position 1 to position 25 means that the deletion cover a sequence of 25 contiguous amino acids . . . .

In one other advantageous embodiment, the invention relates to the use of a hybrid protein comprising the sequence of a first membrane receptor fused at its C-terminus to the N-terminus of a ion channel defined above:
is present in said hybrid protein in its natural configuration, or
is deleted of a number of amino acids ranging from 1 to the total number of amino acids of the region extending from the first amino acid at the N-terminus part of said ion channel to the first amino acids of the first α-helix of the transmembrane domain of said ion channel, or
has an addition, of an additional sequence of a number of amino acids ranging from 1 to the total number of amino acids of the region delimited by the cytoplasmic tail, originating from a second membrane receptor different from said first membrane receptor, preferably said additional sequence corresponding to the C-terminus of said second membrane receptor, or
has, a substitution of a number of amino acids ranging from 1 to the total number of amino acids of the region delimited by the cytoplasmic tail, with a substitute sequence of a number of amino acids ranging from 1 to the total number of amino acids of the region delimited by the cytoplasmic tail from a second membrane receptor different from said first membrane receptor, and a linker is possibly present between the C-terminus of said first membrane receptor and the N-terminus part of said ion channel, said linker being absent in the respective natural configuration of said first membrane receptor and said ion channel.

In one other advantageous embodiment, the invention relates to the use of a hybrid protein comprising the sequence of a first membrane receptor fused at its C-terminus to the N-terminus of a ion channel defined above, wherein:
said ion channel sequence is deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably is deleted from 1 to 49 amino acids at the N-terminus part of said Kir ion channel, or is deleted from 1 to 435 amino acids at the N-terminus of said Kv ion channel, and
said first membrane receptor is present in said hybrid protein in its natural configuration, and
a linker is present between the C-terminus of said first membrane receptor and the N-terminus part of said ion channel, said linker being absent in the natural configuration of said first membrane receptor and said ion channel.

According to the invention, the sequence of hybrid protein comprises, in the amino N-terminus, the sequence of a membrane receptor in its natural configuration, fused to a linker, itself fused to a ion channel deleted sequence being deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably being deleted from 1 to 49 amino acids at the N-terminus part of said Kir ion channel, or being deleted from 1 to 435 amino acids at the N-terminus of said Kv ion channel, in its N-terminus part.

As mentioned above, the deletion of the amino acid sequence in the N-terminus of the ion channel part preferably corresponds to a deletion of contiguous amino acids.

This construction retains the complete sequence of the first membrane receptor and is able to generate an electrical signal, via the ion channel part, when stimulated by a ligand, and to activate signaling pathways normally activated by the first membrane receptor in its natural configuration.

In another advantageous embodiment, the invention relates to the use of a hybrid protein comprising the sequence of a first membrane receptor fused at its C-terminus to the N-terminus of a ion channel defined above, wherein:
said ion channel sequence is deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably is deleted from 1 to 49 amino acids at the N-terminus part of said Kir ion channel, or is deleted from 1 to 435 amino acids at the N-terminus of said Kv ion channel, and
said first membrane receptor is deleted of a number of amino acids ranging from 1 to the total number of amino acids of the region delimited by the cytoplasmic tail, preferably of a number of amino acids ranging from 1 to 100, preferably of a number of amino acids ranging from 1 to 70, preferably of a number of amino acids ranging from 1 to 20, preferably of a number of amino acids ranging from 1 to 15, more preferably of a number of amino acids ranging from 1 to 10, amino acids at the C-terminus, and
a linker is present between the C-terminus of said first membrane receptor and the N-terminus part of said ion channel, said linker being absent in the natural configuration of said first membrane receptor and said ion channel

According to the invention, the sequence of hybrid protein comprises, in the amino N-terminus, the sequence of a membrane receptor deleted in C-terminus part of all the amino acids of the cytoplasmic tail, preferably of 1 to 100, preferably of a number of amino acids ranging from 1 to 70, preferably of a number of amino acids ranging from 1 to 20, preferably of a number of amino acids ranging from 1 to 15, more preferably of a number of amino acids ranging from 1 to 10, fused to a linker, itself fused to an ion channel deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably being deleted from 1 to 49 amino acids at the N-terminus part of said Kir ion channel, or being deleted from 1 to 435 amino acids at the N-terminus of said Kv ion channel, in its N-terminus part.

As mentioned above, the deletion of the amino acid sequence in the N-terminus of the ion channel part preferably corresponds to a deletion of contiguous amino acids.

This construction retains the majority of the sequence of the first membrane receptor and is able to generate an electrical signal, via the ion channel part, when stimulated by a ligand, and to quasi-normally activate signaling pathways normally activated by the first membrane receptor in its natural configuration. This deletion in the first membrane receptor sequence allows to bring close to each other the two sequences comprised in the hybrid protein, and then to induce a best transmission of signal between the first membrane receptor activated by its ligand, and the ion channel able to generate an electrical signal.

In one other particular embodiment, the invention relates to the use of a hybrid protein above-described, wherein said first membrane receptor has a deletion of contiguous amino acids at the C-terminus

The hybrid protein defined above has a contiguous deletion of 1 to 100 amino acids, in the C-terminus of the membrane receptor part.

In another advantageous embodiment, the invention relates to the use of a hybrid protein comprising the sequence of a first membrane receptor fused at its C-terminus to the N-terminus of an ion channel according to described above, wherein:
said ion channel sequence is deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably is deleted from 1 to 49 amino acids at the N-terminus part of said Kir ion channel, or is deleted from 1 to 435 amino acids at the N-terminus of said Kv ion channel, at the N-terminus part of said ion channel, and
an addition, in particular after the last amino acid at the C-terminus of said first membrane receptor, of an additional sequence of a number of amino acids ranging from 1 to the total number of amino acids of the region delimited by the cytoplasmic tail, preferably of a number of amino acids ranging from 1 to to 100, preferably of a number of amino acids ranging from 1 to 70, preferably of a number of amino acids ranging from 1 to 20, preferably of a number of amino acids ranging from 1 to 15, more preferably of a number of amino acids ranging from 1 to 10 amino acids, preferably contiguous, originating from a second membrane receptor different from said first membrane receptor, preferably said additional sequence corresponding to the C-terminus of said second membrane receptor, and
a linker is present between the C-terminus of said first membrane receptor and the N-terminus part of said ion channel, said linker being absent in the natural configuration of said first membrane receptor and said ion channel.

According to the invention, the sequence of hybrid protein comprises, in the amino N-terminus, the sequence of a membrane receptor has in C-terminus part an addition of all the amino acids of the cytoplasmic tail, preferably of 1 to 100, preferably of a number of amino acids ranging from 1 to 70, preferably of a number of amino acids ranging from 1 to 20, preferably of a number of amino acids ranging from 1 to 15, more preferably of a number of amino acids ranging from 1 to 10, fused to a linker, itself fused to a ion channel sequence being deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably being deleted from 1 to 49 amino acids at the N-terminus part of said Kir ion channel, or being deleted from 1 to 435 amino acids at the N-terminus of said Kv ion channel, in its N-terminus part.

The additional sequence is preferably originated from the C-terminus of a second membrane receptor.

This addition in the first membrane receptor sequence enhances the communication between of the sequences comprised in the hybrid protein, and then to induce a best transmission of signal between the first membrane receptor activated by its ligand, and the ion channel able to generate an electrical signal.

In another particular embodiment, the invention relates to the use of a hybrid protein above-described, wherein said first membrane has an addition after the last amino acid at the C-terminus of an additional sequence corresponding to contiguous amino acids from the C-terminus of a second membrane receptor different from said first membrane receptor.

The hybrid protein defined above has an addition of an additional sequence of 1 to 100 contiguous amino acids, originating from the C-terminus of a second membrane receptor.

In another preferred embodiment, the invention also relates to the use of a hybrid protein comprising the sequence of a first membrane receptor fused at its C-terminus to the N-terminus of a ion channel above-mentioned, wherein:
said ion channel sequence is deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably is deleted from 1 to 49 amino acids at the N-terminus part of said Kir ion channel, or is deleted from 1 to 435 amino acids at the N-terminus of said Kv ion channel, and
said first membrane receptor has, at the C-terminus part, a substitution of a number of amino acids ranging from 1 to the total number of amino acids of the region delimited by the cytoplasmic tail, preferably a number of amino acids ranging from 1 to 20 amino acids, preferably a number of amino acids ranging from 1 to 15 amino acids, more preferably a number of amino acids ranging from 1 to 10 amino acids with a substitute sequence of a number of amino acids ranging from 1 to the total number of amino acids of the region delimited by the cytoplasmic tail, preferably a number of amino acids ranging from 1 to 20 amino acids, preferably a number of amino acids ranging from 1 to 15 amino acids, more preferably a number of amino acids ranging from 1 to 10 amino acids from a second membrane receptor different from said first membrane receptor, and
a linker is present between the C-terminus of said first membrane receptor and the N-terminus part of said ion channel, said linker being absent in the natural configuration of said first membrane receptor and said ion channel.

According to the invention, the sequence of hybrid protein comprises, in the N-terminus, the sequence of a membrane receptor with in its C-terminus part a substitution of 1 to all the amino acids of the cytoplasmic tail, preferably from 1 to 20 amino acids, with a substitution sequence comprising 1 to all the amino acids of the cytoplasmic tail, preferably 1 to 20 amino acids, amino acids originating from a second membrane receptor different from said first membrane receptor, fused to a linker, itself fused to an ion channel deleted of a number of amino acids ranging from 1 to the total number of amino acids of the region extending from the first amino acid at the N-terminus part of said ion channel to the first amino acids of the first α-helix of the transmembrane domain of said ion channel, preferably of a number of amino acids ranging from 1 to 29 amino acids, in its N-terminus part.

The substitution sequence is preferably originated from the C-terminus of a second membrane receptor.

This substitution in the first membrane receptor sequence enhances the communication between said two sequences, and induces a best transmission of signal between the first membrane receptor activated by its ligand, and the ion channel able to generate an electrical signal.

In one preferred embodiment, the invention relates to the use of a hybrid protein above-described, wherein said first membrane, at the C-terminus part, has a contiguous substitute sequence originating from the C-terminus of a second membrane receptor different from said first membrane receptor.

The hybrid protein defined above has a substitution of 1 to 20 amino acids, to all the amino acids of the cytoplasmic tail with a substitution sequence of 1 to 20, to all the amino acids of the cytoplasmic tail, contiguous originating from the C-terminus of a second membrane receptor.

In one preferred embodiment, the invention relates to the use of a hybrid protein defined above, comprising a linker present between the C-terminus of said first membrane receptor and the N-terminus part of said ion channel, said linker being absent in the natural configuration of said first membrane receptor and said ion channel, in particular comprising or constituted by six contiguous glycine residues, represented by the following sequence: -G-G-G-G-G-G- (SEQ ID NO 196).

In another advantageous embodiment, the invention relates to the use of a hybrid protein comprising the sequence of a first membrane receptor fused at its C-terminus to the N-terminus of a ion channel above-described, wherein:
said ion channel sequence is deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably is deleted from 1 to 49 amino acids at the N-terminus part of said Kir ion channel, or is deleted from 1 to 435 amino acids at the N-terminus of said Kv ion channel, and
said first membrane receptor is present in said hybrid protein in its natural configuration, and
said hybrid protein having no linker between the C-terminus of said first membrane receptor and the N-terminus part of said ion channel.

The invention also relates, in one advantageous embodiment, to the use of a hybrid protein comprising the sequence of a first membrane receptor fused at its C-terminus to the N-terminus of a ion channel defined above, wherein:
said ion channel sequence is deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably is deleted from 1 to 49 amino acids at the N-terminus part of said Kir ion channel, or is deleted from 1 to 435 amino acids at the N-terminus of said Kv ion channel, and
said first membrane receptor is deleted of a number of amino acids ranging from 1 to the total number of amino acids of the region delimited by the cytoplasmic tail, preferably of a number of amino acids ranging from 1 to 20, preferably of a number of amino acids ranging from 1 to 15, more preferably of a number of amino acids ranging from 1 to 10, amino acids at the C-terminus, and
said hybrid protein has no linker between the C-terminus of said first membrane receptor and the N-terminus part of said ion channel.

This deletion in the first membrane receptor sequence allows to bring close to each other the two sequences comprised in the hybrid protein, and then to induce a best transmission of signal between the first membrane receptor activated by its ligand, and the ion channel able to generate an electrical signal.

In another specific embodiment, the invention relates to the use of a hybrid protein above-mentioned, wherein said first membrane receptor has a deletion of 2 to many contiguous amino acids at the C-terminus, such deletion possibly extending from the C-terminus extremity to the first transmembrane helix of the receptor.

Also, in one other particular embodiment, the invention relates to the use of a hybrid protein comprising the sequence of a first membrane receptor fused at its C-terminus to the N-terminus of a ion channel previously described, wherein:
said ion channel sequence is deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably is deleted from 1 to 49 amino acids at the N-terminus part of said Kir ion channel, or is deleted from 1 to 435 amino acids at the N-terminus of said Kv ion channel, and
an addition, in particular after the last amino acid at the C-terminus of said first membrane receptor, of an additional sequence of a number of amino acids ranging from 1 to the total number of amino acids of the region delimited by the cytoplasmic tail, preferably of a number of amino acids ranging from 1 to 20 amino acids, preferably of a number of amino acids ranging from 1 to 15 amino acids, more preferably of a number of amino acids ranging from 1 to 10 amino acids, preferably contiguous, originating from a second membrane receptor different from said first membrane receptor, preferably said additional sequence corresponding to the C-terminus of said second membrane receptor, and
said hybrid protein has no linker between the C-terminus of said first membrane receptor and the N-terminus part of said ion channel.

This addition in the first membrane receptor sequence enhances the communication between receptor and channel in the hybrid protein, and induces a best transmission of signal between the first membrane receptor activated by its ligand, and the ion channel able to generate an electrical signal.

Moreover, in another advantageous embodiment, the invention relates to the use of a hybrid protein described herein, wherein said first membrane has an addition after the last amino acid at the C-terminus of an additional sequence corresponding to contiguous amino acids from the C-terminus of a second membrane receptor different from said first membrane receptor.

In another advantageous embodiment, the invention relates to the use of a hybrid protein comprising the sequence of a first membrane receptor fused at its C-terminus to the N-terminus of a ion channel above-mentioned, wherein:
said ion channel sequence is deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably is deleted from 1 to 49 amino acids at the N-terminus part of said Kir ion channel, or is deleted from 1 to 435 amino acids at the N-terminus of said Kv ion channel, and
said first membrane receptor has, at the C-terminus part, a substitution of a number of amino acids ranging from 1 to the total number of amino acids of the region delimited by the cytoplasmic tail, preferably a number of amino acids ranging from 1 to 20 amino acids, preferably a number of amino acids ranging from 1 to 15 amino acids, more preferably a number of amino acids ranging from 1 to 10 amino acids with an substitute sequence of a number of amino acids ranging from 1 to the total number of amino acids of the region delimited by the cytoplasmic tail, preferably a number of amino acids ranging from 1 to 20 amino acids, preferably a number of amino acids ranging from 1 to 15 amino acids, more preferably a number of amino acids ranging from 1 to 10 amino acids from a second membrane receptor different from said first membrane receptor, and
said hybrid protein has no linker between the C-terminus of said first membrane receptor and the N-terminus part of said ion channel.

This substitution in the first membrane receptor sequence allows to bring close to each other and enhance the communication between the two sequences comprised in the hybrid protein, and then to induce a best transmission of signal between the first membrane receptor activated by its ligand, and the ion channel able to generate an electrical signal.

In another advantageous embodiment, the invention relates to the use of a hybrid protein defined above, wherein said first membrane, at the C-terminus part, has a contiguous substitute sequence originating from the C-terminus of a second membrane receptor different from said first membrane receptor.

In another advantageous embodiment, the invention relates to the use of a hybrid protein above defined, comprising a tag, in particular chosen among the group consisting in:
Hemaglutinin Tag, in particular comprising or consisting in SEQ ID NO 158,
Poly Arginine Tag, in particular comprising or consisting in SEQ ID NO 160,
Poly Histidine Tag, in particular comprising or consisting in SEQ ID NO 162,
Myc Tag, in particular comprising or consisting in SEQ ID NO 164,
Strep Tag, in particular comprising or consisting in SEQ ID NO 166,
Flag Tag, in particular comprising or consisting in SEQ ID NO 168,
S-Tag, in particular comprising or consisting in SEQ ID NO 170,
HAT Tag, in particular comprising or consisting in SEQ ID NO 172,
3× Flag Tag, in particular comprising or consisting in SEQ ID NO 174,
Calmodulin-binding peptide Tag, in particular comprising or consisting in SEQ ID NO 176,
VSVG Tag, in particular comprising or consisting in SEQ ID NO 178,
SBP Tag, in particular comprising or consisting in SEQ ID NO 180,
Chitin-binding domain Tag, in particular comprising or consisting in SEQ ID NO 182,
GST Tag, in particular comprising or consisting in SEQ ID NO 184,
Maltose-Binding protein Tag, in particular comprising or consisting in SEQ ID NO 186,
GFP Tag, in particular comprising or consisting in SEQ ID NO 188,
RFP Tag, in particular comprising or consisting in SEQ ID NO 190,
YFP Tag, in particular comprising or consisting in SEQ ID NO 192, and
CFP Tag, in particular comprising or consisting in SEQ ID NO 194.

Thus, the invention relates, in one preferred embodiment, to the use of a hybrid protein above defined, comprising a tag, in particular chosen among the group consisting in SEQ ID NO 2q, q varying from 79 to 97.

All the mentioned tags have been characterized previously, and antibodies recognizing each of them are commercially available.

In another advantageous embodiment, the invention relates to the use as defined above wherein said ion channel is chosen among:
the Kir potassium channels selected from the group comprising the potassium channels Kir1.1, Kir2.1, Kir2.2, Kir2.3, Kir2.4, Kir3.1, Kir3.2, Kir3.3, Kir3.4, Kir4.1, Kir4.2, Kir5.1, Kir6.1, Kir6.2 and Kir7.1, or
the Kv potassium channels selected from the group comprising the potassium channels Kv1.1, Kv1.2, Kv1.3, Kv1.4, Kv1.5, Kv1.6, Kv1.7, Kv1.8, Kv2.1, Kv2.2, Kv3.1, Kv3.2, Kv3.3, Kv3.4, Kv4.1, Kv4.2, Kv4.3, Kv5.1, Kv6.1, Kv6.2, Kv6.3, Kv6.4, Kv7.1, Kv7.2, Kv7.3, Kv7.4, Kv7.5, Kv8.1, Kv8.2, Kv9.1, Kv9.2, Kv9.3, Kv10.1, Kv10.2, Kv11.1, Kv11.2, Kv11.3, Kv12.1, Kv12. and Kv12.3.

In another advantageous embodiment, the invention relates to the use of a hybrid protein above-described, wherein said first and second membrane receptor sequence is the sequence of a membrane receptor belonging to the family of G-protein coupled receptors (GPCR) class A receptors.

In another advantageous embodiment, the invention relates to the use of a hybrid protein mentioned above, wherein said first and second membrane receptors are GPCR receptors chosen among the group comprising:
muscarinic receptor, in particular the human muscarinic M2 receptor, in particular comprising or constituted by SEQ ID NO 10,
adrenergic receptor, in particular the human β2-adrenergic receptor, in particular comprising or constituted by SEQ ID NO 12,
dopaminergic receptor, in particular the human dopaminergic long D2 receptor, in particular comprising or constituted by SEQ ID NO 14,
dopaminergic receptor, in particular the human dopaminergic D3 receptor, in particular comprising or constituted by SEQ ID NO 229
serotonergic receptor, in particular the human 5HT 1α receptor, in particular comprising or constituted by SEQ ID NO 16,
canabinoïd receptor, in particular the human CB 1 receptor, in particular comprising or constituted by SEQ ID NO 230

In another advantageous embodiment, the invention relates to the use of a hybrid protein described above, wherein said first and second membrane receptor sequence is the sequence of a membrane receptor belonging to the family of chemokine receptors (CR).

Chemokine receptors are cytokine receptors found on the surface of certain cells, which interact with a type of cytokine called chemokine. There have been 19 distinct chemokine receptors described in mammals. They each have a 7 transmembrane (7TM) structure and are coupled to G-protein for signal transduction within a cell, making them members of a large protein family of G protein-coupled receptors. Following interaction with their specific chemokine ligands, chemokine receptors trigger an increase in intracellular calcium (Ca2+) ions (calcium signaling) within the cell. This causes cell responses, including the onset of a process known as chemotaxis that traffics the cell to a desired location within the organism. Chemokine receptors are divided into different families, CXC chemokine receptors, CC chemokine receptors, CX3C chemokine receptors and XC chemokine receptors that correspond to the 4 distinct subfamilies of chemokines they bind.

In another advantageous embodiment, the invention relates to the use of a hybrid protein defined above, wherein said chemokine receptor is chosen among the group comprising:
CXCR4 receptor, in particular the human CXCR4 receptor, in particular comprising or constituted by SEQ ID NO 18,
CCR5 receptor, in particular the human CCR5 receptor, in particular comprising or constituted by SEQ ID NO 20, and
CCR2 receptor, in particular the human CCR2 receptor, in particular comprising or constituted by SEQ ID NO 231.

In a specific embodiment, the invention relates to specific hybrid fusion proteins as mentioned above and detailed in the following Table ibis:

TABLE 1
bis represents all the hybrids proteins disclosed and used in the invention. Membrane receptor part of the hybrid protein is
indicated in grey, and ion channel part of the hybrid protein is indicated in white. -ΔN refers to the deletion of the N terminus part of
the ion channel.

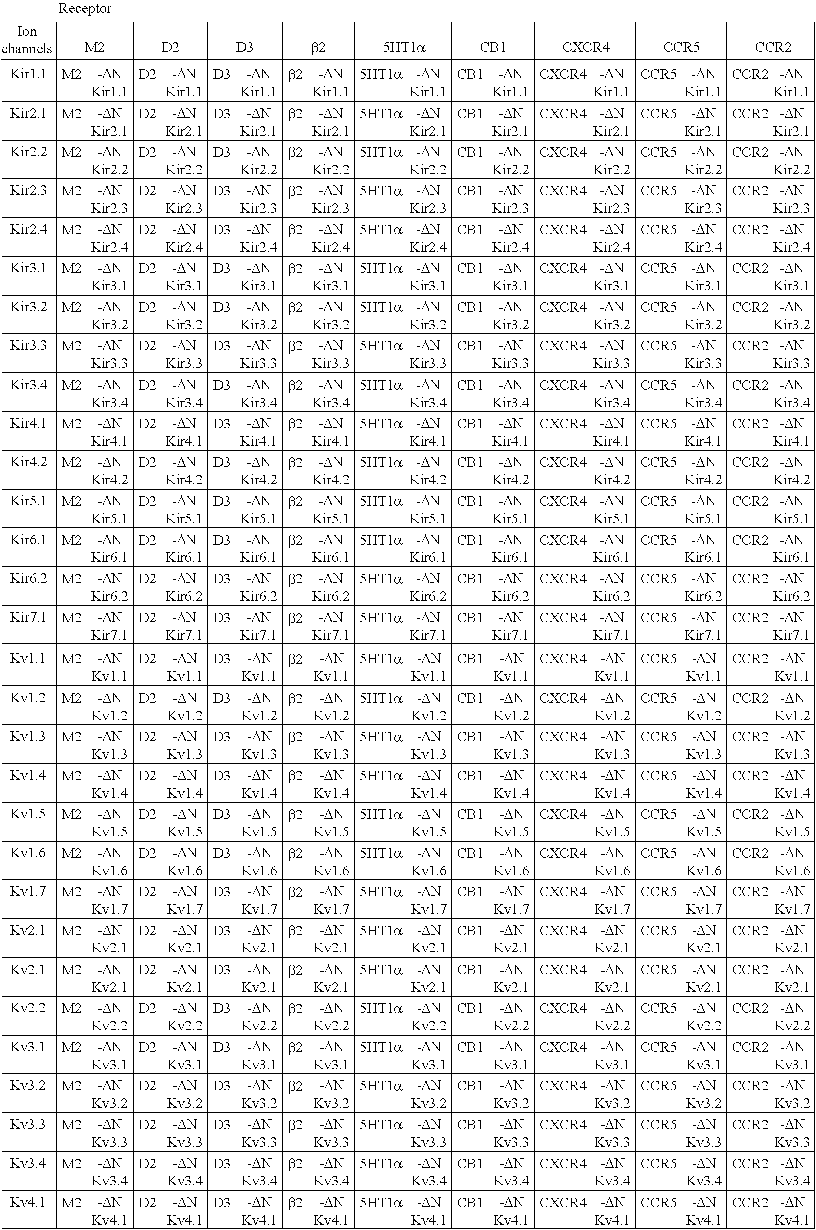

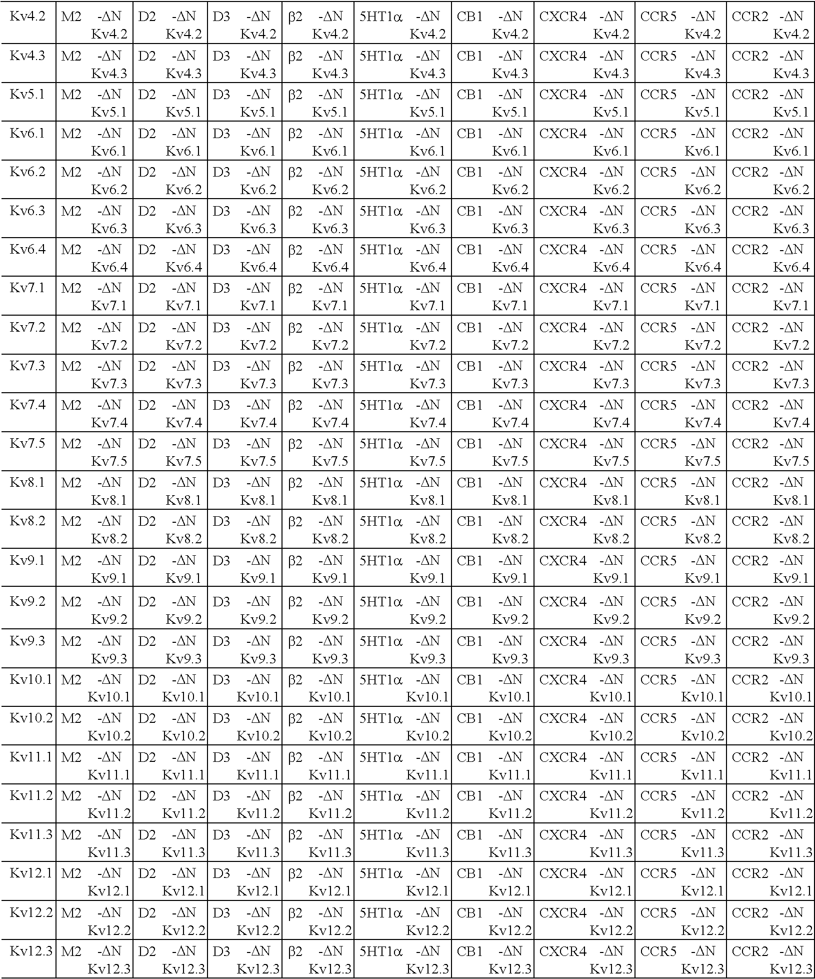

In another advantageous embodiment, the invention relates to the use as defined above wherein said ion channel is Kir6.2.

Kir6.2 is a ATP dependent potassium channel (K_{ATP} channel), relatively simple, well-studied K⁺ channel that has the unique signature of being inhibited by intracellular ATP. This convenient feature provides a straightforward means to identify the channel and control its open probability [Moreau C, Prost A L, Derand R, Vivaudou M (2005) SUR, ABC proteins targeted by K_{ATP} channel openers. J Mol Cell Cardiol. 38:951-63; Nichols C G (2006) K_{ATP} channels as molecular sensors of cellular metabolism. Nature. 440:470-6].

In another advantageous embodiment, the invention relates to the use of a hybrid protein defined above, wherein said ion channel is the murine or human Kir6.2, and in particular comprises or is constituted by the amino acid sequence SEQ ID NO 2.

Preferably in the invention the hybrid protein comprises or is constituted by the murine Kir6.2 as ion channel part.

In another advantageous embodiment, the invention relates to the use of a hybrid protein defined above comprising:

1—a membrane receptor sequence, in its natural configuration, fused to the N-terminus deleted sequence of Kir6.2, or
2—a membrane receptor sequence, in its natural configuration, fused to a linker sequence, said linker sequence being fused to the N-terminus deleted sequence of Kir6.2, or
3—a membrane receptor sequence, deleted in its C-terminus part, fused to the N-terminus deleted sequence of Kir6.2, or
4—a membrane receptor sequence, deleted in its C-terminus part, fused to a linker sequence, said linker sequence being fused to the N-terminus deleted sequence of Kir6.2, or
5—a membrane receptor sequence, having an addition at the C-terminus of an additional sequence from a second membrane receptor, fused to the N-terminus deleted sequence of Kir6.2, or
6—a membrane receptor sequence, having an addition at the C-terminus of an additional sequence from a second membrane receptor, fused to a linker sequence, said linker sequence being fused to the N-terminus deleted sequence of Kir6.2, or
7—a membrane receptor sequence, having a substitution at the C-terminus with a substitution sequence from a second membrane receptor, fused to the N-terminus deleted sequence of Kir6.2, or
8—a membrane receptor sequence, having an substitution at the C-terminus with a substitution sequence from a second membrane receptor, fused to the N-terminus deleted sequence of Kir6.2, fused to a linker sequence, said linker sequence being fused to the N-terminus deleted sequence of Kir6.2.

In another advantageous embodiment, the invention relates to the use of a hybrid protein defined above, wherein the Kir6.2 ion channel sequence is deleted in the C-terminus part, in particular deleted from 1 to 36 of its 36 last amino acids at the C-terminus, and in particular comprises or is constituted by the amino acid sequence SEQ ID NO 4.

Preferably in the invention the hybrid protein comprises or is constituted by the murine Kir6.2 ion channel as ion channel part, wherein 26, or 36 amino acids have been deleted of the C-terminus part of said Kir6.2 ion channel (include endoplasmic retention signal (RKR) mutations) [Zerangue N, Schwappach B, Jan Y N, Jan L Y (1999) A new ER trafficking signal regulates the subunit stoichiometry of plasma membrane K_{ATP} channels. Neuron. 22:537-48]. The 2 to 36 amino acids deleted are preferably contiguous. This deletion preferably begin at position 354 of the amino acid sequence of the mouse Kir6.2 protein. The endosplamic retention signal can also be abolished by single or multiple substitutions of the amino acids (RKR) by all other type of residues.

In another advantageous embodiment, the invention relates to the use of a hybrid protein defined above, wherein the sequence of the Kir6.2 ion channel sequence contains an insertion of 11 amino acids, preferably contiguous, in the external loop of said Kir6.2, and in particular comprises or is constituted by SEQ ID NO 6 or by SEQ ID NO 8.

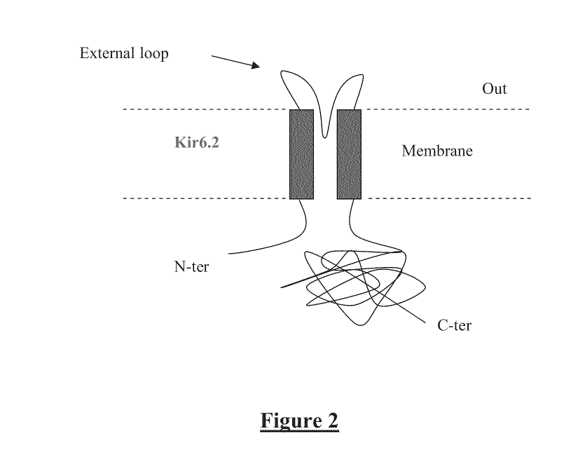

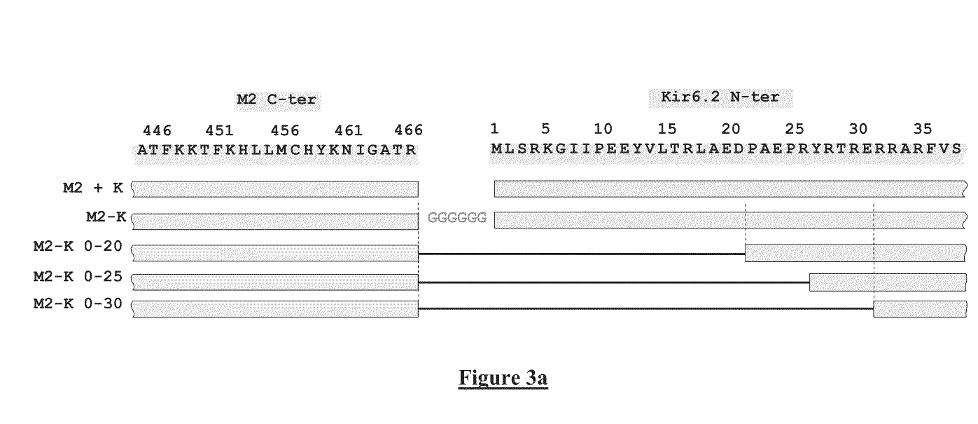

According to the invention, the hybrid protein comprises or is constituted by the murine Kir6.2 ion channel as ion channel part wherein 11 amino acids have been inserted in the external loop of Kir6.2. The external loop is represented in FIG. 2. The external loop is defined by the amino acids comprised between the positions 91 to 119. The numbering is defined from the sequence of Kir6.2 in its natural configuration, as mentioned above. This fragment of Kir6.2 corresponds to the following sequence:

(SEQ ID NO 232)
91-WLIAFAHGDLAPGEGTNVPCVTSIHSFSS-119.

When the 11 amino acids sequence is inserted into the loop sequence, the sequence becomes:

(SEQ ID NO 233)
91-WLIAFAHGDLYAYMEKGITDLAPGEGTNVPCVTSIHSFSS-130.

This region corresponds also to the region wherein the tag sequence above-mentioned can be inserted. The tag sequence and/or 11-amino acids insertion in the external loop of Kir6.2 protein enhances the size of external loop. It allows to provide a best accessibility to antibodies able to detect either external loop sequence, or tag sequence.

The tag insertion and/or 11-amino acids insertion does not modify the conformation of ion channel, and said ion channel remains able to generate, with the same ability as the ion channel in its natural configuration, an electrical current.

In one particular embodiment of the invention, when the HA tag sequence (SEQ ID NO 158) is inserted in the amino acid sequence of the loop containing the 11 amino acids insertion (SEQ ID NO 198), the sequence becomes:

(SEQ ID NO 234)
91-WLIAFAHGDLYAYMEKGITDLAPYPYDVPDYAGEGTNVPCVTSIH
SFSS-139.

In another advantageous embodiment, the invention relates to the use of a hybrid protein described above, wherein said hybrid protein is chosen among the group consisting in SEQ ID NO 2q, q varying from 15 to 70 and from 99 to 102.

By SEQ ID NO 2q, q varying from 15 to 70, it is means all the following sequences: SEQ ID NO 30, SEQ ID NO 32, SEQ ID NO 34, SEQ ID NO 36, SEQ ID NO 38, SEQ ID NO 40, SEQ ID NO 42, SEQ ID NO 44, SEQ ID NO 46, SEQ ID NO 48, SEQ ID NO 50, SEQ ID NO 52, SEQ ID NO 54, SEQ ID NO 56, SEQ ID NO 58, SEQ ID NO 60, SEQ ID NO 62, SEQ ID NO 64, SEQ ID NO 66, SEQ ID NO 68, SEQ ID NO 70, SEQ ID NO 72, SEQ ID NO 74, SEQ ID NO 76, SEQ ID NO 78, SEQ ID NO 80, SEQ ID NO 82, SEQ ID NO 84, SEQ ID NO 86, SEQ ID NO 88, SEQ ID NO 90, SEQ ID NO 92, SEQ ID NO 94, SEQ ID NO 96, SEQ ID NO 98, SEQ ID NO 100, SEQ ID NO 102, SEQ ID NO 104, SEQ ID NO 106, SEQ ID NO 108, SEQ ID NO 110, SEQ ID NO 112, SEQ ID NO 114, SEQ ID NO 116, SEQ ID NO 118, SEQ ID NO 120, SEQ ID NO 122, SEQ ID NO 124, SEQ ID NO 126, SEQ ID NO 128, SEQ ID NO 130, SEQ ID NO 132, SEQ ID NO 134, SEQ ID NO 136, SEQ ID NO 138 and SEQ ID NO 140.

The above mentioned sequences correspond to particular constructions, the characteristics of which are indicated in the following table 1.

| Construct name | TAG | Δ C-RM | Add C-RM | Δ N-Kir | Add +11 ELoop | Δ C Kir | Protein NO |
|---|---|---|---|---|---|---|---|
| M2-KΔ 0 0 + 6G HA | Y (HA) | No | No | No | Y | Y | SEQ ID NO 22 |
| M2-K 0 0 + 6G HA | Y (HA) | No | No | No | Y | No | SEQ ID NO 24 |
| M2-K 0 0 + 6G | No | No | No | No | No | No | SEQ ID NO 26 |
| M2-KΔ 0 0 + 6G | No | No | No | No | No | Y | SEQ ID NO 28 |
| M2-KΔ 0 − 20 HA | Y (HA) | No | No | Y (−20) | Y | Y | SEQ ID NO 30 |
| M2-K 0 − 20 HA | Y (HA) | No | No | Y (−20) | Y | No | SEQ ID NO 32 |
| M2-K 0 − 20 | No | No | No | Y (−20) | No | No | SEQ ID NO 34 |
| M2-KΔ 0 − 20 | No | No | No | Y (−20) | No | Y | SEQ ID NO 36 |
| M2-KΔ −5 − 20 HA | Y (HA) | Y (−5) | No | Y (−20) | Y | Y | SEQ ID NO 38 |
| M2-K −5 − 20 HA | Y (HA) | Y (−5) | No | Y (−20) | Y | No | SEQ ID NO 40 |
| M2-K −5 − 20 | No | Y (−5) | No | Y (−20) | No | No | SEQ ID NO 42 |
| M2-KΔ −5 − 20 | No | Y (−5) | No | Y (−20) | No | Y | SEQ ID NO 44 |
| M2-KΔ 0 − 25 HA | Y (HA) | No | No | Y (−25) | Y | Y | SEQ ID NO 46 |
| M2-K 0 − 25 HA | Y (HA) | No | No | Y (−25) | Y | No | SEQ ID NO 48 |
| M2-K 0 − 25 | No | No | No | Y (−25) | No | No | SEQ ID NO 50 |
| M2-KΔ 0 − 25 | No | No | No | Y (−25) | No | Y | SEQ ID NO 52 |
| M2-KΔ 0 − 30 HA | Y (HA) | No | No | Y (−30) | Y | Y | SEQ ID NO 54 |
| M2-K 0 − 30 HA | Y (HA) | No | No | Y (−30) | Y | No | SEQ ID NO 56 |
| M2-K 0 − 30 | No | No | No | Y (−30) | No | No | SEQ ID NO 58 |
| M2-KΔ 0 − 30 | No | No | No | Y (−30) | No | Y | SEQ ID NO 60 |
| D2-KΔ 0 − 25 HA | Y (HA) | No | No | Y (−25) | Y | Y | SEQ ID NO 62 |
| D2-K 0 − 25 HA | Y (HA) | No | No | Y (−25) | Y | No | SEQ ID NO 64 |
| D2-K 0 − 25 | No | No | No | Y (−25) | No | No | SEQ ID NO 66 |
| D2-KΔ 0 − 25 | No | No | No | Y (−25) | No | Y | SEQ ID NO 68 |
| D2-KΔ 0 − 16 HA | Y (HA) | No | No | Y (−16) | Y | Y | SEQ ID NO 70 |
| D2-K 0 − 16 HA | Y (HA) | No | No | Y (−16) | Y | No | SEQ ID NO 72 |
| D2-K 0 − 16 | No | No | No | Y (−16) | No | No | SEQ ID NO 74 |
| D2-KΔ 0 − 16 | No | No | No | Y (−16) | No | Y | SEQ ID NO 76 |
| D2-KΔ +9_{M2} − 25 HA | Y (HA) | No | Y (+9-M2) | Y (−25) | Y | Y | SEQ ID NO 78 |
| D2-K +9_{M2} − 25 HA | Y (HA) | No | Y (+9-M2) | Y (−25) | Y | No | SEQ ID NO 80 |
| D2-K +9_{M2} − 25 | No | No | Y (+9-M2) | Y (−25) | No | No | SEQ ID NO 82 |
| D2-KΔ +9_{M2} − 25 | No | No | Y (+9-M2) | Y (−25) | No | Y | SEQ ID NO 84 |
| D2-KΔ 0 − 30 HA | Y (HA) | No | No | Y (−30) | Y | Y | SEQ ID NO 86 |
| D2-K 0 − 30 HA | Y (HA) | No | No | Y (−30) | Y | No | SEQ ID NO 88 |
| D2-K 0 − 30 | No | No | No | Y (−30) | No | No | SEQ ID NO 90 |
| D2-KΔ 0 − 30 | No | No | No | Y (−30) | No | Y | SEQ ID NO 92 |
| D3-KΔ 0 − 25 HA | Y (HA) | No | No | Y (−25) | Y | Y | SEQ ID NO 204 |
| B2-K 0 − 25 HA | Y (HA) | No | No | Y (−25) | Y | No | SEQ ID NO 94 |
| B2-KΔ 0 − 25 | No | No | No | Y (−25) | No | Y | SEQ ID NO 96 |
| B2-K 0 − 25 | No | No | No | Y (−25) | No | No | SEQ ID NO 98 |
| B2-KΔ 0 − 25 HA | Y (HA) | No | No | Y (−25) | Y | Y | SEQ ID NO 100 |
| B2-K −63 − 25 HA | Y (HA) | Y (−63) | No | Y (−25) | Y | No | SEQ ID NO 102 |
| B2-KΔ −63 − 25 HA | Y (HA) | Y (−63) | No | Y (−25) | Y | Y | SEQ ID NO 104 |
| B2-K −63 − 25 | No | Y (−63) | No | Y (−25) | No | No | SEQ ID NO 106 |
| B2-KΔ −63 − 25 | No | Y (−63) | No | Y (−25) | No | Y | SEQ ID NO 108 |
| B2-K 0 − 30 HA | Y (HA) | No | No | Y (−30) | Y | No | SEQ ID NO 110 |
| B2-KΔ 0 − 30 HA | Y (HA) | No | No | Y (−30) | Y | Y | SEQ ID NO 112 |
| B2-K 0 − 30 | No | No | No | Y (−30) | No | No | SEQ ID NO 114 |
| B2-KΔ 0 − 30 | No | No | No | Y (−30) | No | Y | SEQ ID NO 116 |
| B2-KΔ −73 − 25 HA | Y (HA) | Y (−73) | No | Y (−25) | Y | Y | SEQ ID NO 198 |
| 5HT1a-K 0 − 25 HA | Y (HA) | No | No | Y (−25) | Y | No | SEQ ID NO 118 |
| 5HT1a-KΔ 0 − 25 HA | Y (HA) | No | No | Y (−25) | Y | Y | SEQ ID NO 120 |
| 5HT1a-K 0 − 25 | No | No | No | Y (−25) | No | No | SEQ ID NO 122 |
| 5HT1a- KΔ 0 − 25 | No | No | No | Y (−25) | No | Y | SEQ ID NO 124 |
| 5HT1a-K +4_{M2} − 25 HA | Y (HA) | No | Y (+4-M2) | Y (−25) | Y | No | SEQ ID NO 126 |
| 5HT1a-KΔ +4_{M2} − 25 HA | Y (HA) | No | Y (+4-M2) | Y (−25) | Y | Y | SEQ ID NO 128 |
| 5HT1a-K +4_{M2} − 25 | No | No | Y (+4-M2) | Y (−25) | No | No | SEQ ID NO 130 |
| 5HT1a-KΔ +4_{M2} − 25 | No | No | Y (+4-M2) | Y (−25) | No | Y | SEQ ID NO 132 |
| 5HT1a-K 0 − 30 HA | Y (HA) | No | No | Y (−30) | Y | No | SEQ ID NO 134 |
| 5HT1a-KΔ 0 − 30 HA | Y (HA) | No | No | Y (−30) | Y | Y | SEQ ID NO 136 |
| 5HT1a-K 0 − 30 | No | No | No | Y (−30) | No | No | SEQ ID NO 138 |
| 5HT1a-KΔ 0 − 30 | No | No | No | Y (−30) | No | Y | SEQ ID NO 140 |
| CB1-KΔ 0 − 25 HA | Y (HA) | No | No | Y (−25) | Y | Y | SEQ ID NO 200 |
| CB1-KΔ −48 − 25 HA | Y (HA) | Y (−48) | No | Y (−25) | Y | Y | SEQ ID NO 202 |

Table 1 describes the characteristics of the hybrid proteins described in the invention. Y means Yes and corresponds to the presence of the above mentioned element, No corresponds to the absence of the above mentioned element. Y (−X) means that X amino acids have been deleted. Y (+X − Z) means that X amino acids have been added, said X amino acids originating from the Z protein.
TAG indicates the presence of a TAG sequence in the external loop of Kir6.2 sequence. Δ C-RM indicates the presence or absence of a deletion in the C-terminus part of membrane receptor. Add C-RM indicates the presence or absence of an additional sequence to the C-terminus part of membrane receptor. Δ C Kir indicates the presence or absence of 36 amino acids in the C-terminus of Kir6.2 sequence. Add +11 ELoop indicates the presence of an addition of 11 amino acids in the external loop of Kir6.2 sequence. Δ N-Kir indicates the presence or absence of a deletion in the N-terminus part of Kir6.2. M2 means muscarinic receptor, D2 means dopaminergic receptor D2, D3 means dopaminergic receptor D3, B2 means β2-adrenergic receptor, 5HT1a means 5HT1α receptor and CB1 means CB1 canabinoid receptor.

In another embodiment, the invention relates to the use of a hybrid protein defined above, wherein said hybrid protein is chosen among the group consisting in SEQ ID NO 2q, q varying from 71 to 78 and SEQ ID NO 206.

By SEQ ID NO 2q, q varying from 71 to 78, it is defined in the invention the following sequences: SEQ ID NO 142, SEQ ID NO 144, SEQ ID NO 146, SEQ ID NO 148, SEQ ID NO 150, SEQ ID NO 152, SEQ ID NO 154 and SEQ ID NO 156.

The above mentioned sequences correspond to particular constructions, the characteristics of which are indicated in the following table 2.

| Construct name | TAG | Δ C-RM | Add C-RM | Δ N-Kir | Add +11 ELoop | Δ C Kir | Protein NO |
|---|---|---|---|---|---|---|---|
| CXCR4-K 0 − 25 HA | Y (HA) | No | No | Y (−25) | Y | No | SEQ ID NO 142 |
| CXCR4-KΔ 0 − 25 HA | Y (HA) | No | No | Y (−25) | Y | Y | SEQ ID NO 144 |
| CXCR4-K 0 − 25 | No | No | No | Y (−25) | No | No | SEQ ID NO 146 |
| CXCR4-KΔ 0 − 25 | No | No | No | Y (−25) | No | Y | SEQ ID NO 148 |
| CCR5-K 0 − 25 HA | Y (HA) | No | No | Y (−25) | Y | No | SEQ ID NO 150 |
| CCR5-KΔ 0 − 25 HA | Y (HA) | No | No | Y (−25) | Y | Y | SEQ ID NO 152 |
| CCR5-K 0 − 25 | No | No | No | Y (−25) | No | No | SEQ ID NO 154 |
| CCR5-KΔ 0 − 25 | No | No | No | Y (−25) | No | Y | SEQ ID NO 156 |
| CCR2-KΔ 0 − 25 HA | Y (HA) | No | No | Y (−25) | Y | Y | SEQ ID NO 206 |

Table 2 describes the characteristics of the hybrid proteins described in the invention. Y means Yes.
TAG indicates the presence of a TAG sequence in the external loop of Kir6.2 sequence. Δ C-RM indicates the presence or absence of a deletion in the C-terminus part of membrane receptor. Add C-RM indicates the presence or absence of an additional sequence to the C-terminus part of membrane receptor. Δ C Kir indicates the presence or absence of 36 amino acids in the C-terminus of Kir6.2 sequence. Add +11 ELoop indicates the presence of an addition of 11 amino acids in the external loop of Kir6.2 sequence. Δ N-Kir indicates the presence or absence of a deletion in the N-terminus part of Kir6.2. CXCR4 means CXCR4 receptor, CCR5 means CCR5 receptor and CCR2 means CCR2 receptor.

In another preferred embodiment, the invention relates to the use of a hybrid protein above defined, wherein said hybrid protein is chosen among the group consisting in SEQ ID NO 32, SEQ ID NO 40, SEQ ID NO 48, SEQ ID NO 56, SEQ ID NO 64, SEQ ID NO 72, SEQ ID NO 80, SEQ ID NO 88 and SEQ ID NO 94.

In another embodiment, the invention relates to the use of a hybrid protein defined above, said hybrid protein being inserted in a membrane, preferably a membrane comprising lipids.

According to the invention, “membrane” is defined as a compound separating two conditions.

Usually, membrane consists of polymers and permits selective transport of material. Membrane may contain auxiliary parts for mechanical support.

The driving force of the material transport is given by concentration, pressure, electrical or chemical gradient across the membrane.

The applications depend on the type of functionality incorporated in the membrane, which can be based on size-exclusion, chemical affinity or electrostatics.

In the invention said membrane does not allow the passive diffusion of ions, and the ionic concentration gradient establishes a potential differential, which allows the current generation.

Also according to the invention, the membrane can be a biological membrane.

A biological membrane or biomembrane is an enclosing or separating amphipathic layer that acts as a barrier within or around a cell. It is, almost invariably, a lipid bilayer, composed of a double layer of lipid-class molecules, specifically phospholipids, with occasional proteins intertwined, some of which function as channels.

Such membranes typically define enclosed spaces or compartments in which cells may maintain a chemical or biochemical environment that differs from the outside.

The most important feature of a biomembrane is that it is a selectively-permeable structure. This means that the size, charge, and other chemical properties of the atoms and molecules attempting to cross it will determine whether they succeed in doing so. Selective permeability is essential for effective separation of a cell from its surroundings. Biological membranes also have certain mechanical or elastic properties.

If a particle is too large or otherwise unable to cross the membrane by itself, but is still needed by a cell, it could go through one of the protein channels for example.

The hybrid protein according to the invention is prepared from host cells expressing the hybrid protein as defined above, by using standard recombinant protein expression techniques, which are well-known in the art. Alternatively, the hybrid protein is purified and incorporated in an artificial membrane, by using standard techniques as described for example in Silvius J R, 1992, Annu. Rev. Biophys. Biomol. Struct., 21, 323-348.

In another particular embodiment, the invention relates to the use of a nucleic acid molecule coding for a hybrid protein defined above.

Particularly in one embodiment, the invention relates to the use of nucleic acid molecules as defined above, wherein said nucleic molecules have a nucleic acid sequence chosen among the group consisting SEQ ID NO 2q-1, q varying from 15 to 78 and from 99 to 103.

Said nucleic acid sequences defined above by SEQ ID NO 2q-1, q varying from 15 to 78, correspond to the following sequences: SEQ ID NO 29, SEQ ID NO 31, SEQ ID NO 33, SEQ ID NO 35, SEQ ID NO 37, SEQ ID NO 39, SEQ ID NO 41, SEQ ID NO 43, SEQ ID NO 45, SEQ ID NO 47, SEQ ID NO 49, SEQ ID NO 51, SEQ ID NO 53, SEQ ID NO 55, SEQ ID NO 57, SEQ ID NO 59, SEQ ID

NO 61, SEQ ID NO 63, SEQ ID NO 65, SEQ ID NO 67, SEQ ID NO 69, SEQ ID NO 71, SEQ ID NO 73, SEQ ID NO 75, SEQ ID NO 77, SEQ ID NO 79, SEQ ID NO 81, SEQ ID NO 83, SEQ ID NO 85, SEQ ID NO 87, SEQ ID NO 89, SEQ ID NO 91, SEQ ID NO 93, SEQ ID NO 95, SEQ ID NO 97, SEQ ID NO 99, SEQ ID NO 101, SEQ ID NO 103, SEQ ID NO 105, SEQ ID NO 107, SEQ ID NO 109, SEQ ID NO 111, SEQ ID NO 113, SEQ ID NO 115, SEQ ID NO 117, SEQ ID NO 119, SEQ ID NO 121, SEQ ID NO 123, SEQ ID NO 125, SEQ ID NO 127, SEQ ID NO 129, SEQ ID NO 131, SEQ ID NO 133, SEQ ID NO 135, SEQ ID NO 137, SEQ ID NO 139, SEQ ID NO 141, SEQ ID NO 143, SEQ ID NO 145, SEQ ID NO 147, SEQ ID NO 149, SEQ ID NO 151, SEQ ID NO 153 and SEQ ID NO 155 and SEQ ID NO 197, SEQ ID NO 199, SEQ ID NO 201, SEQ ID NO 203 and SEQ ID NO 205

These nucleic acid sequences are defined such as the protein with amino acid sequence SEQ ID NO 30 is coded by the nucleic acid molecule having the sequence SEQ ID NO 29, the protein with amino acid sequence SEQ ID NO 32 is coded by the nucleic acid molecule having the sequence SEQ ID NO 31, the protein with amino acid sequence SEQ ID NO 34 is coded by the nucleic acid molecule having the sequence SEQ ID NO 33, etc. . . .

The following table 3 recapitulate the correspondence between protein sequence and their coding sequence. The name of protein or peptide is also indicated.

| Construct name | Protein NO | DNA NO |
|---|---|---|
| mouse Kir6.2 channel | SEQ ID NO 2 | SEQ ID NO 1 |
| mouse Kir6.2 channel Δ36 | SEQ ID NO 4 | SEQ ID NO 3 |
| mouse Kir6.2 channel + 11 | SEQ ID NO 6 | SEQ ID NO 5 |
| mouse Kir6.2 channel + 11 Δ36 | SEQ ID NO 8 | SEQ ID NO 7 |
| human M2 Muscarinic receptor | SEQ ID NO 10 | SEQ ID NO 9 |
| human β2 Adrenergic receptor | SEQ ID NO 12 | SEQ ID NO 11 |
| human long D2 Dopaminergic | SEQ ID NO 14 | SEQ ID NO 13 |
| receptor | | |
| human 5HT1α Serotoninergic | SEQ ID NO 16 | SEQ ID NO 15 |
| receptor | | |
| human CXCR4 Chemokine | SEQ ID NO 18 | SEQ ID NO 17 |
| receptor | | |
| human CCR5 Chemokine | SEQ ID NO 20 | SEQ ID NO 19 |
| receptor | | |
| M2-KΔ 0 0 + 6G HA | SEQ ID NO 22 | SEQ ID NO 21 |
| M2-K 0 0 + 6G HA | SEQ ID NO 24 | SEQ ID NO 23 |
| M2-K 0 0 + 6G | SEQ ID NO 26 | SEQ ID NO 25 |
| M2-KΔ 0 0 + 6G | SEQ ID NO 28 | SEQ ID NO 27 |
| M2-KΔ 0 − 20 HA | SEQ ID NO 30 | SEQ ID NO 29 |
| M2-K 0 − 20 HA | SEQ ID NO 32 | SEQ ID NO 31 |
| M2-K 0 − 20 | SEQ ID NO 34 | SEQ ID NO 33 |
| M2-KΔ 0 − 20 | SEQ ID NO 36 | SEQ ID NO 35 |
| M2-KΔ −5 − 20 HA | SEQ ID NO 38 | SEQ ID NO 37 |
| M2-K −5 − 20 HA | SEQ ID NO 40 | SEQ ID NO 39 |
| M2-K −5 − 20 | SEQ ID NO 42 | SEQ ID NO 41 |
| M2-KΔ −5 − 20 | SEQ ID NO 44 | SEQ ID NO 43 |
| M2-KΔ 0 − 25 HA | SEQ ID NO 46 | SEQ ID NO 45 |
| M2-K 0 − 25 HA | SEQ ID NO 48 | SEQ ID NO 47 |
| M2-K 0 − 25 | SEQ ID NO 50 | SEQ ID NO 49 |
| M2-KΔ 0 − 25 | SEQ ID NO 52 | SEQ ID NO 51 |
| M2-KΔ 0 − 30 HA | SEQ ID NO 54 | SEQ ID NO 53 |
| M2-K 0 − 30 HA | SEQ ID NO 56 | SEQ ID NO 55 |
| M2-K 0 − 30 | SEQ ID NO 58 | SEQ ID NO 57 |
| M2-KΔ 0 − 30 | SEQ ID NO 60 | SEQ ID NO 59 |
| D2-KΔ 0 − 25 HA | SEQ ID NO 62 | SEQ ID NO 61 |
| D2-K 0 − 25 HA | SEQ ID NO 64 | SEQ ID NO 63 |
| D2-K 0 − 25 | SEQ ID NO 66 | SEQ ID NO 65 |
| D2-KΔ 0 − 25 | SEQ ID NO 68 | SEQ ID NO 67 |
| D2-KΔ 0 − 16 HA | SEQ ID NO 70 | SEQ ID NO 69 |
| D2-K 0 − 16 HA | SEQ ID NO 72 | SEQ ID NO 71 |
| D2-K 0 − 16 | SEQ ID NO 74 | SEQ ID NO 73 |
| D2-KΔ 0 − 16 | SEQ ID NO 76 | SEQ ID NO 75 |
| D2-KΔ +9_{M2} − 25 HA | SEQ ID NO 78 | SEQ ID NO 77 |
| D2-K +9_{M2} − 25 HA | SEQ ID NO 80 | SEQ ID NO 79 |
| D2-K +9_{M2} − 25 | SEQ ID NO 82 | SEQ ID NO 81 |
| D2-KΔ +9_{M2} − 25 | SEQ ID NO 84 | SEQ ID NO 83 |
| D2-KΔ 0 − 30 HA | SEQ ID NO 86 | SEQ ID NO 85 |
| D2-K 0 − 30 HA | SEQ ID NO 88 | SEQ ID NO 87 |
| D2-K 0 − 30 | SEQ ID NO 90 | SEQ ID NO 89 |
| D2-KΔ 0 − 30 | SEQ ID NO 92 | SEQ ID NO 91 |
| B2-K 0 − 25 HA | SEQ ID NO 94 | SEQ ID NO 93 |
| B2-KΔ 0 − 25 | SEQ ID NO 96 | SEQ ID NO 95 |
| B2-K 0 − 25 | SEQ ID NO 98 | SEQ ID NO 97 |
| B2-KΔ 0 − 25 HA | SEQ ID NO 100 | SEQ ID NO 99 |
| B2-K −63 − 25 HA | SEQ ID NO 102 | SEQ ID NO 101 |
| B2-KΔ −63 − 25 HA | SEQ ID NO 104 | SEQ ID NO 103 |
| B2-K −63 − 25 | SEQ ID NO 106 | SEQ ID NO 105 |
| B2-KΔ −63 − 25 | SEQ ID NO 108 | SEQ ID NO 107 |
| B2-K 0 − 30 HA | SEQ ID NO 110 | SEQ ID NO 109 |
| B2-KΔ 0 − 30 HA | SEQ ID NO 112 | SEQ ID NO 111 |
| B2-K 0 − 30 | SEQ ID NO 114 | SEQ ID NO 113 |
| B2-KΔ 0 − 30 | SEQ ID NO 116 | SEQ ID NO 115 |
| 5HT1a-K 0 − 25 HA | SEQ ID NO 118 | SEQ ID NO 117 |
| 5HT1a-KΔ 0 − 25 HA | SEQ ID NO 120 | SEQ ID NO 119 |
| 5HT1a-K 0 − 25 | SEQ ID NO 122 | SEQ ID NO 121 |
| 5HT1a-KΔ 0 − 25 | SEQ ID NO 124 | SEQ ID NO 123 |
| 5HT1a-K +4_{M2} − 25 HA | SEQ ID NO 126 | SEQ ID NO 125 |
| 5HT1a-KΔ +4_{M2} − 25 HA | SEQ ID NO 128 | SEQ ID NO 127 |
| 5HT1a-K +4_{M2} − 25 | SEQ ID NO 130 | SEQ ID NO 129 |
| 5HT1a-KΔ +4_{M2} − 25 | SEQ ID NO 132 | SEQ ID NO 131 |
| 5HT1a-K 0 − 30 HA | SEQ ID NO 134 | SEQ ID NO 133 |
| 5HT1a-KΔ 0 − 30 HA | SEQ ID NO 136 | SEQ ID NO 135 |
| 5HT1a-K 0 − 30 | SEQ ID NO 138 | SEQ ID NO 137 |
| 5HT1a-KΔ 0 − 30 | SEQ ID NO 140 | SEQ ID NO 139 |
| CXCR4-K 0 − 25 HA | SEQ ID NO 142 | SEQ ID NO 141 |
| CXCR4-KΔ 0 − 25 HA | SEQ ID NO 144 | SEQ ID NO 143 |
| CXCR4-K 0 − 25 | SEQ ID NO 146 | SEQ ID NO 145 |
| CXCR4-KΔ 0 − 25 | SEQ ID NO 148 | SEQ ID NO 147 |
| CCR5-K 0 − 25 HA | SEQ ID NO 150 | SEQ ID NO 149 |
| CCR5-KΔ 0 − 25 HA | SEQ ID NO 152 | SEQ ID NO 151 |
| CCR5-K 0 − 25 | SEQ ID NO 154 | SEQ ID NO 153 |
| CCR5-KΔ 0 − 25 | SEQ ID NO 156 | SEQ ID NO 155 |
| Hemaglutinin (HA) | SEQ ID NO 158 | SEQ ID NO 157 |
| Poly-Arginine (5-6, usually 5) | SEQ ID NO 160 | SEQ ID NO 159 |
| Poly-Histidine (2-10, usually 6) | SEQ ID NO 162 | SEQ ID NO 161 |
| c-Myc | SEQ ID NO 164 | SEQ ID NO 163 |
| Strep-tag II | SEQ ID NO 166 | SEQ ID NO 165 |
| Flag | SEQ ID NO 168 | SEQ ID NO 167 |
| S-tag | SEQ ID NO 170 | SEQ ID NO 169 |
| HAT- | SEQ ID NO 172 | SEQ ID NO 171 |
| 3x FLAG | SEQ ID NO 174 | SEQ ID NO 173 |
| Calmodulin-binding peptide | SEQ ID NO 176 | SEQ ID NO 175 |
| VSVG | SEQ ID NO 178 | SEQ ID NO 177 |
| SBP | SEQ ID NO 180 | SEQ ID NO 179 |
| Chitin-binding domain | SEQ ID NO 182 | SEQ ID NO 181 |
| Glutathione S-transferase | SEQ ID NO 184 | SEQ ID NO 183 |
| Maltose-binding protein | SEQ ID NO 186 | SEQ ID NO 185 |
| GFP | SEQ ID NO 188 | SEQ ID NO 187 |
| RFP | SEQ ID NO 190 | SEQ ID NO 189 |
| YFP | SEQ ID NO 192 | SEQ ID NO 191 |
| CFP | SEQ ID NO 194 | SEQ ID NO 193 |
| Poly Glu | SEQ ID NO 196 | SEQ ID NO 195 |
| B2-KΔ −73 − 25 HA | SEQ ID NO 198 | SEQ ID NO 197 |
| CB1-KΔ 0 − 25 HA | SEQ ID NO 200 | SEQ ID NO 199 |
| CB1-KΔ −48 − 25 HA | SEQ ID NO 202 | SEQ ID NO 201 |
| D3-KΔ 0 − 25 HA | SEQ ID NO 204 | SEQ ID NO 203 |
| CCR-KΔ 0 − 25 HA | SEQ ID NO 206 | SEQ ID NO 205 |

Table 3 indicates the correspondence between protein and coding sequence (DNA)

In another particular embodiment, the invention relates to the use of a vector comprising a nucleic acid molecule mentioned above, and comprising elements allowing the expression of said nucleic acid molecule in host cells.

By “elements allowing the expression of said nucleic acid molecule” it is meant in the invention nucleic acid sequences such as, promoter, terminator, polyadenylation sites and all the necessary sequence that are needed for a correct expression in cell.

Vectors of the invention are preferably expression vectors, wherein a sequence encoding a hybrid protein of the invention is placed under control of appropriate transcriptional and translational control elements. These vectors may be obtained and introduced in a host cell by the well-known recombinant DNA and genetic engineering techniques.

In another advantageous embodiment, the invention relates to the use of a vector defined above, wherein said host cells are chosen among bacteria, yeast, mammalian cells, insect cells or amphibian oocytes.

Preferably bacteria are E. coli. Preferred yeast in the invention is, but is not limited to, S. cerevisiae or Sc. pombe or Pichia pastoris. Mammalian cells are defined in the invention by all the mammalian cell lines commonly used in the art, for in vivo experiments, such as human cell lines, murine cell lines, rodent cell lines . . . .

Said host cell may be obtained by transfection with the hybrid protein, the polynucleotide (DNA, RNA) encoding said hybrid protein, or with the expression vector comprising said polynucleotide, as defined above.

The invention also relates to a hybrid protein comprising the sequence of a first membrane receptor fused at its C-terminus to the N-terminus of an ion channel, and possibly containing a linker between the C-terminus of said first membrane receptor and the N-terminus part of said ion channel, said linker being absent in the natural configuration of said first membrane receptor and said ion channel,
said ion channel sequence being deleted of a number of amino acids ranging from 1 to the total number of amino acids of the region extending from the first amino acid at the N-terminus part of said ion channel to the first amino acids of the first α-helix of the transmembrane domain of said ion channel, possibly containing a tag sequence,
said first membrane receptor being liable to present in its extracellular domain a mutation allowing the specific interaction with a ligand different from the ligand that interacts with the first membrane receptor in its natural configuration,
said first membrane receptor being liable to present in its cytoplasmic tail, said cytoplasmic tail being a sequence extending from the last amino acids after the transmembrane domain to the last amino acid of said first membrane receptor,
   a) a deletion of a number of amino acids ranging from 1 to the total number of amino acids of the region delimited by the cytoplasmic tail, preferably of a number of amino acids ranging from 1 to 20, preferably of a number of amino acids ranging from 1 to 15, more preferably of a number of amino acids ranging from 1 to 10, amino acids at the C-terminus, and/or
   b) an addition, in particular after the last amino acid at the C-terminus of said first membrane receptor, of an additional sequence of a number of amino acids ranging from 1 to the total number of amino acids of the region delimited by the cytoplasmic tail, preferably of a number of amino acids ranging from 1 to 20 amino acids, preferably of a number of amino acids ranging from 1 to 15 amino acids, more preferably of a number of amino acids ranging from 1 to 10 amino acids, preferably contiguous, originating from a second membrane receptor different from said first membrane receptor, preferably said additional sequence corresponding to the C-terminus of said second membrane receptor, and/or
   c) a substitution of a number of amino acids ranging from 1 to the total number of amino acids of the region delimited by the cytoplasmic tail, preferably a number of amino acids ranging from 1 to 20 amino acids, preferably a number of amino acids ranging from 1 to 15 amino acids, more preferably a number of amino acids ranging from 1 to 10 amino acids with an substitute sequence of a number of amino acids ranging from 1 to the total number of amino acids of the region delimited by the cytoplasmic tail, preferably a number of amino acids ranging from 1 to 20 amino acids, preferably a number of amino acids ranging from 1 to 15 amino acids, more preferably a number of amino acids ranging from 1 to 10 amino acids from a second membrane receptor different from said first membrane receptor,
      said hybrid protein being such that said ion channel retains the property of electrical current generation of said ion channel in its natural configuration, and that said first membrane receptor retains the ability to interact with the ligand of said first membrane receptor in its natural configuration.

In a preferred embodiment, the invention relates to a hybrid protein comprising or consisting in
a. the sequence of a first membrane receptor, said first membrane receptor belonging to the G-protein coupled receptors (GPCR) class A family, covalently fused at its C-terminus to
b. the N-terminus sequence of an ion channel, said ion channel belonging to the potassium channel families selected from the inwardly rectifying potassium channels (Kir) family and the voltage-dependent potassium channels (Kᵥ) family,
c. and possibly containing a linker sequence between the C-terminus of said first membrane receptor and the N-terminus part of said ion channel, said linker being absent in the natural configuration of said first membrane receptor and said ion channel,
said ion channel sequence being deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably being deleted from 1 to 49 amino acids at the N-terminus part of said Kir ion channel, or being deleted from 1 to 435 amino acids at the N-terminus of said Kv ion channel,
said ion channel possibly containing a tag sequence,
said first membrane receptor being liable to present in its extracellular domain a mutation allowing the specific interaction with a ligand different from the ligand that interacts with the first membrane receptor in its natural configuration,
said first membrane receptor being liable to present in its cytoplasmic tail, said cytoplasmic tail being a sequence delimited by the first amino acid after the last amino acid of the transmembrane helix and the last amino acid of said first membrane receptor, in particular being liable to present in the 100 amino acids in its C-terminus part
   a deletion of a number of amino acids ranging from 1 to the total number of amino acids of the region delimited by the cytoplasmic tail, preferably of a number of amino acids ranging from 1 to 100, preferably of a number of amino acids ranging from 1 to 70, preferably of a number of amino acids ranging from 1 to 20 preferably of a number of amino acids ranging from 1 to 15, more preferably of a number of amino acids ranging from 1 to 10, amino acids at the C-terminus, provided that said deletion does not affect the transmembrane amino acid sequence of said membrane receptor and/or
   an addition, in particular after the last amino acid at the C-terminus of said first membrane receptor, of an additional sequence of a number of amino acids ranging from 1 to the total number of amino acids of the region delimited by the cytoplasmic tail, preferably of a number of amino acids ranging from 1 to 100 amino acids, preferably of a number of amino acids ranging from 1 to 70, preferably of a number of amino acids ranging from 1 to 20 amino acids, preferably of a number of amino acids ranging from 1 to 15 amino acids, more preferably of a number of amino acids ranging from 1 to 10 amino acids, preferably contiguous, originating from a second membrane receptor different from said first membrane receptor, preferably said additional sequence corresponding to the C-terminus of said second membrane receptor, and/or
   a substitution of a number of amino acids ranging from 1 to the total number of amino acids of the region delimited by the cytoplasmic tail, preferably a number of amino acids ranging from 1 to 20 amino acids, preferably a number of amino acids ranging from 1 to 15 amino acids, more preferably a number of amino acids ranging from 1 to 10 amino acids with an substitute sequence of a number of amino acids ranging from 1 to the total number of amino acids of the region delimited by the cytoplasmic tail, preferably a number of amino acids ranging from 1 to 20 amino acids, preferably a number of amino acids ranging from 1 to 15 amino acids, more preferably a number of amino acids ranging from 1 to 10 amino acids from a second membrane receptor different from said first membrane receptor
      said hybrid protein being such that said ion channel retains the property of electrical current generation of said ion channel in its natural configuration, and that said first membrane receptor retains the ability to interact with the ligand of said first membrane receptor in its natural configuration.

In an advantageous embodiment, the invention relates to a hybrid protein comprising or consisting in:
a. the sequence of a first membrane receptor, said first membrane receptor belonging to the G-protein coupled receptors (GPCR) class A family, covalently fused at its C-terminus to
b. the N-terminus sequence of an ion channel, said ion channel belonging to the potassium channel families selected from the inwardly rectifying potassium channels (Kir) family and the voltage-dependent potassium channels (Kᵥ) family,
   said ion channel sequence being deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel,
   said ion channel possibly containing a tag sequence,
   said first membrane receptor being liable to present in its cytoplasmic tail at least one mutation, said cytoplasmic tail being a sequence delimited by the first amino acid after the last amino acid of the transmembrane helix and the last amino acid of said first membrane receptor,
a deletion of a number of amino acids ranging from 1 to the total number of amino acids of the region delimited by the cytoplasmic tail, and/or
an addition, of an additional sequence of a number of amino acids ranging from 1 to the total number of amino acids of the region delimited by the cytoplasmic tail, originating from a second membrane receptor different from said first membrane receptor, preferably said additional sequence corresponding to the C-terminus of said second membrane receptor and/or
a substitution of a number of amino acids ranging from 1 to the total number of amino acids of the region delimited by the cytoplasmic tail, with an substitute sequence of a number of amino acids ranging from 1 to the total number of amino acids of the region delimited by the cytoplasmic tail from a second membrane receptor different from said first membrane receptor
   said hybrid protein being such that said ion channel retains the property of electrical current generation of said ion channel in its natural configuration, and that said first membrane receptor retains the ability to interact with the ligand of said first membrane receptor in its natural configuration.

In one particular embodiment, the invention also relates to a hybrid protein comprising the sequence of a first membrane receptor fused at its C-terminus to the N-terminus of a ion channel, and possibly containing a linker between the C-terminus of said first membrane receptor and the N-terminus part of said ion channel, said linker being absent in the natural configuration of said first membrane receptor and said ion channel,
said ion channel sequence being deleted of a number of amino acids ranging from 1 to 29 amino acids at the N-terminus part of said ion channel, possibly containing a tag sequence,
said first membrane receptor being liable to present in the 70 amino acids in its C-terminus part
   of a number of amino acids ranging from Ito 20, preferably of a number of amino acids ranging from 1 to 15, more preferably of a number of amino acids ranging from 1 to 10, amino acids at the C-terminus, and/or
   an addition, in particular after the last amino acid at the C-terminus of said first membrane receptor, of an additional sequence of a number of amino acids ranging from 1 to 20 amino acids, preferably of a number of amino acids ranging from 1 to 15 amino acids, more preferably of a number of amino acids ranging from 1 to 10 amino acids, preferably contiguous, originating from a second membrane receptor different from said first membrane receptor, preferably said additional sequence corresponding to the C-terminus of said second membrane receptor, and/or
   a substitution of a number of amino acids ranging from 1 to 20 amino acids, preferably a number of amino acids ranging from 1 to 15 amino acids, more preferably a number of amino acids ranging from 1 to 10 amino acids with an substitute sequence of a number of amino acids ranging from 1 to 20 amino acids, preferably a number of amino acids ranging from 1 to 15 amino acids, more preferably a number of amino acids ranging from 1 to 10 amino acids from a second membrane receptor different from said first membrane receptor,
said hybrid protein being such that said ion channel retains the property of electrical current generation of said ion channel in its natural configuration, and that said first membrane receptor retains the ability to interact with the ligand of said first membrane receptor in its natural configuration.

This hybrid protein such as defined in the invention is new.
In one advantageous embodiment, the invention relates to a hybrid protein defined above, wherein said ion channel sequence said ion channel sequence being deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably being deleted from 1 to 49 amino acids at the N-terminus part of said Kir ion channel, or being deleted from 1 to 435 amino acids at the N-terminus of said Kv ion channel, the numbering being defined from the first amino acid at the N-terminus of said ion channel in its natural configuration.

In one advantageous embodiment, the invention relates to a hybrid protein comprising the sequence of a first membrane receptor fused at its C-terminus to the N-terminus of a ion channel defined above, wherein:
said ion channel sequence is deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably being deleted from 1 to 49 amino acids at the N-terminus part of said Kir ion channel, or being deleted from 1 to 435 amino acids at the N-terminus of said Kv ion channel, at the N-terminus part of said ion channel, and
said first membrane receptor is present in said hybrid protein in its natural configuration, and
a linker is present between the C-terminus of said first membrane receptor and the N-terminus part of said ion channel, said linker being absent in the natural configuration of said first membrane receptor and said ion channel.

In one advantageous embodiment, the invention relates to a hybrid protein comprising the sequence of a first membrane receptor fused at its C-terminus to the N-terminus of a ion channel defined above, wherein:
said ion channel sequence deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably being deleted from 1 to 49 amino acids at the N-terminus part of said Kir ion channel, or being deleted from 1 to 435 amino acids at the N-terminus of said Kv ion channel, and
said first membrane receptor is deleted of a number of amino acids ranging from 1 to the total number of amino acids of the region delimited by the cytoplasmic tail, preferably of a number of amino acids ranging from 1 to 100, preferably from 1 to 20, preferably of a number of amino acids ranging from 1 to 15, more preferably of a number of amino acids ranging from 1 to 10, amino acids at the C-terminus, and
a linker is present between the C-terminus of said first membrane receptor and the N-terminus part of said ion channel, said linker being absent in the natural configuration of said first membrane receptor and said ion channel.

In one advantageous embodiment, the invention relates to a hybrid protein according described herein, wherein said first membrane has a deletion of contiguous amino acids at the C-terminus

In one advantageous embodiment, the invention relates to a hybrid protein comprising the sequence of a first membrane receptor fused at its C-terminus to the N-terminus of a ion channel defined above, wherein:
said ion channel sequence is deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably being deleted from 1 to 49 amino acids at the N-terminus part of said Kir ion channel, or being deleted from 1 to 435 amino acids at the N-terminus of said Kv ion channel, and
an addition, in particular after the last amino acid at the C-terminus of said first membrane receptor, of an additional sequence of a number of amino acids ranging from 1 to the total number of amino acids of the region delimited by the cytoplasmic tail, preferably of a number of amino acids ranging from 1 to 100, preferably from 1 to 20 amino acids, preferably of a number of amino acids ranging from 1 to 15 amino acids, more preferably of a number of amino acids ranging from 1 to 10 amino acids, preferably contiguous, originating from a second membrane receptor different from said first membrane receptor, preferably said additional sequence corresponding to the C-terminus of said second membrane receptor, and
a linker is present between the C-terminus of said first membrane receptor and the N-terminus part of said ion channel, said linker being absent in the natural configuration of said first membrane receptor and said ion channel.

In one advantageous embodiment, the invention relates to a hybrid protein defined above, wherein said first membrane has an addition after the last amino acid at the C-terminus of an additional sequence corresponding to contiguous amino acids from the C-terminus of a second membrane receptor different from said first receptor

In one advantageous embodiment, the invention relates to a hybrid protein comprising the sequence of a first membrane receptor fused at its C-terminus to the N-terminus of a ion channel defined above, wherein:
said ion channel sequence is deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably being deleted from 1 to 49 amino acids at the N-terminus part of said Kir ion channel, or being deleted from 1 to 435 amino acids at the N-terminus of said Kv ion channel, and
said first membrane receptor has, at the C-terminus part, a substitution of a number of amino acids ranging from 1 to the total number of amino acids of the region delimited by the cytoplasmic tail, preferably a number of amino acids ranging from 1 to 20 amino acids, preferably a number of amino acids ranging from 1 to 15 amino acids, more preferably a number of amino acids ranging from 1 to 10 amino acids with an substitute sequence of a number of amino acids ranging from 1 to the total number of amino acids of the region delimited by the cytoplasmic tail, preferably a number of amino acids ranging from 1 to 20 amino acids, preferably a number of amino acids ranging from 1 to 15 amino acids, more preferably a number of amino acids ranging from 1 to 10 amino acids from a second membrane receptor different from said first membrane receptor, and
a linker is present between the C-terminus of said first membrane receptor and the N-terminus part of said ion channel, said linker being absent in the natural configuration of said first membrane receptor and said ion channel.

In one advantageous embodiment, the invention relates to a hybrid protein defined above, wherein said first membrane, at the C-terminus part, has a contiguous substitute sequence originating from the C-terminus of a second membrane receptor different from said first membrane receptor.

In one advantageous embodiment, the invention relates to a hybrid protein comprising the sequence of a first membrane receptor fused at its C-terminus to the N-terminus of a ion channel defined above, wherein said first membrane receptor
a. is present in said hybrid protein in its natural configuration, or
b. is deleted of a number of amino acids ranging from 1 to the total number of amino acids of the region extending from the first amino acid at the N-terminus part of said ion channel to the first amino acids of the first α-helix of the transmembrane domain of said ion channel, or
c. has an addition, of an additional sequence of a number of amino acids ranging from 1 to the total number of amino acids of the region delimited by the cytoplasmic tail, originating from a second membrane receptor different from said first membrane receptor, preferably said additional sequence corresponding to the C-terminus of said second membrane receptor, or
d. has, a substitution of a number of amino acids ranging from 1 to the total number of amino acids of the region delimited by the cytoplasmic tail, with an substitute sequence of a number of amino acids ranging from 1 to the total number of amino acids of the region delimited by the cytoplasmic tail from a second membrane receptor different from said first membrane receptor,
   and a linker is possibly present between the C-terminus of said first membrane receptor and the N-terminus part of said ion channel, said linker being absent in the natural configuration of said first membrane receptor and said ion channel.

In one advantageous embodiment, the invention relates to a hybrid protein defined above, comprising a linker present between the C-terminus of said first membrane receptor and the N-terminus part of said ion channel, said linker being absent in the natural configuration of said first membrane receptor and said ion channel, in particular comprising or constituted by six contiguous glycine residues, represented by the following sequence: -G-G-G-G-G-G- (SEQ ID NO 196).

In one advantageous embodiment, the invention relates to a hybrid protein comprising the sequence of a first membrane receptor fused at its C-terminus to the N-terminus of a ion channel defined above, wherein:
said ion channel sequence is deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably being deleted from 1 to 49 amino acids at the N-terminus part of said Kir ion channel, or being deleted from 1 to 435 amino acids at the N-terminus of said Kv ion channel, at the N-terminus part of said ion channel, and
said first membrane receptor is present in said hybrid protein in its natural configuration, and
said hybrid protein has no linker between the C-terminus of said first membrane receptor and the N-terminus part of said ion channel.

In one advantageous embodiment, the invention relates to a hybrid protein comprising the sequence of a first membrane receptor fused at its C-terminus to the N-terminus of a ion channel defined above, wherein:
said ion channel sequence is deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably being deleted from 1 to 49 amino acids at the N-terminus part of said Kir ion channel, or being deleted from 1 to 435 amino acids at the N-terminus of said Kv ion channel, and
said first membrane receptor is deleted of a number of amino acids ranging from 1 to the total number of amino acids of the region delimited by the cytoplasmic tail, preferably of a number of amino acids ranging from 1 to 100, preferably from 1 to 20, of a number of amino acids ranging from 1 to 15, more of a number of amino acids ranging from 1 to 10, amino acids at the C-terminus, and
said hybrid protein has no linker between the C-terminus of said first membrane receptor and the N-terminus part of said ion channel.

In one advantageous embodiment, the invention relates to a hybrid protein defined above, wherein said first membrane has a deletion of contiguous amino acids at the C-terminus.

In one advantageous embodiment, the invention relates to a hybrid protein comprising the sequence of a first membrane receptor fused at its C-terminus to the N-terminus of a ion channel defined above, wherein:
said ion channel sequence is deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably being deleted from 1 to 49 amino acids at the N-terminus part of said Kir ion channel, or being deleted from 1 to 435 amino acids at the N-terminus of said Kv ion channel, and
an addition, in particular after the last amino acid at the C-terminus of said first membrane receptor, of an additional sequence of a number of amino acids ranging from 1 to the total number of amino acids of the region delimited by the cytoplasmic tail, preferably of a number of amino acids ranging from 1 to 100, preferably from 1 to 20 amino acids, preferably of a number of amino acids ranging from 1 to 15 amino acids, more preferably of a number of amino acids ranging from 1 to 10 amino acids, preferably contiguous, originating from a second membrane receptor different from said first membrane receptor, preferably said additional sequence corresponding to the C-terminus of said second membrane receptor, and
said hybrid protein has no linker between the C-terminus of said first membrane receptor and the N-terminus part of said ion channel.

In one advantageous embodiment, the invention relates to a hybrid protein defined above, wherein said first membrane has an addition after the last amino acid at the C-terminus of an additional sequence corresponding to contiguous amino acids from the C-terminus of a second membrane receptor different from said first receptor

In one advantageous embodiment, the invention relates to a hybrid protein comprising the sequence of a first membrane receptor fused at its C-terminus to the N-terminus of a ion channel defined above, wherein:
said ion channel sequence is deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably being deleted from 1 to 49 amino acids at the N-terminus part of said Kir ion channel, or being deleted from 1 to 435 amino acids at the N-terminus of said Kv ion channel, and
said first membrane receptor has, at the C-terminus part, a substitution of a number of amino acids ranging from 1 to the total number of amino acids of the region delimited by the cytoplasmic tail, preferably a number of amino acids ranging from 1 to 20 amino acids, preferably a number of amino acids ranging from 1 to 15 amino acids, more preferably a number of amino acids ranging from 1 to 10 amino acids with an substitute sequence of a number of amino acids ranging from 1 to the total number of amino acids of the region delimited by the cytoplasmic tail, preferably a number of amino acids ranging from 1 to 20 amino acids, preferably a number of amino acids ranging from 1 to 15 amino acids, more preferably a number of amino acids ranging from 1 to 10 amino acids from a second membrane receptor different from said first membrane receptor, and
said hybrid protein has no linker between the C-terminus of said first membrane receptor and the N-terminus part of said ion channel.

In one advantageous embodiment, the invention relates to a hybrid protein defined above, wherein said first membrane, at the C-terminus part, has a contiguous substitute sequence originating from the C-terminus of a second membrane receptor different from said first membrane receptor.

In one advantageous embodiment, the invention relates to a hybrid protein defined above, comprising a tag, in particular chosen among the group consisting in:
Hemaglutinin Tag, in particular comprising or consisting in SEQ ID NO 158,
Poly Arginine Tag, in particular comprising or consisting in SEQ ID NO 160,
Poly Histidine Tag, in particular comprising or consisting in SEQ ID NO 162,
Myc Tag, in particular comprising or consisting in SEQ ID NO 164,
Strep Tag, in particular comprising or consisting in SEQ ID NO 166,
Flag Tag, in particular comprising or consisting in SEQ ID NO 168,
S-Tag, in particular comprising or consisting in SEQ ID NO 170,
HAT Tag, in particular comprising or consisting in SEQ ID NO 172,
3×Flag Tag, in particular comprising or consisting in SEQ ID NO 174,
Calmodulin-binding peptide Tag, in particular comprising or consisting in SEQ ID NO 176,
VSVG Tag, in particular comprising or consisting in SEQ ID NO 178,
SBP Tag, in particular comprising or consisting in SEQ ID NO 180,
Chitin-binding domain Tag, in particular comprising or consisting in SEQ ID NO 182,
GST Tag, in particular comprising or consisting in SEQ ID NO 184,
Maltose-Binding protein Tag, in particular comprising or consisting in SEQ ID NO 186,
GFP Tag, in particular comprising or consisting in SEQ ID NO 188,
RFP Tag, in particular comprising or consisting in SEQ ID NO 190,
YFP Tag, in particular comprising or consisting in SEQ ID NO 192, and
CFP Tag, in particular comprising or consisting in SEQ ID NO 194.

In another advantageous embodiment, the invention relates to a hybrid protein as defined above wherein said ion channel is chosen among:
the Kir potassium channels selected from the group comprising the potassium channels Kir1.1, Kir2.1, Kir2.2, Kir2.3, Kir2.4, Kir3.1, Kir3.2, Kir3.3, Kir3.4, Kir4.1, Kir4.2, Kir5.1, Kir6.1, Kir6.2 and Kir7.1, or

the Kv potassium channels selected from the group comprising the potassium channels Kv1.1, Kv1.2, Kv1.3, Kv1.4, Kv1.5, Kv1.6, Kv1.7, Kv1.8, Kv2.1, Kv2.2, Kv3.1, Kv3.2, Kv3.3, Kv3.4, Kv4.1, Kv4.2, Kv4.3, Kv5.1, Kv6.1, Kv6.2, Kv6.3, Kv6.4, Kv7.1, Kv7.2, Kv7.3, Kv7.4, Kv7.5, Kv8.1, Kv8.2, Kv9.1, Kv9.2, Kv9.3, Kv10.1, Kv10.2, Kv11.1, Kv11.2, Kv11.3, Kv12.1, Kv12. and Kv12.3.

In one advantageous embodiment, the invention relates to a hybrid protein defined above, wherein said first membrane receptor sequence is the sequence of a membrane receptor belonging to the family of G protein coupled receptors (GPCR) class A.

In one advantageous embodiment, the invention relates to a hybrid protein defined above, wherein said first and second membrane receptors are GPCR class A receptors chosen among the group comprising:
muscarinic receptor, in particular the human muscarinic M2 receptor, in particular comprising or constituted by SEQ ID NO 10,
adrenergic receptor, in particular the human β2-adrenergic receptor, in particular comprising or constituted by SEQ ID NO 12,
dopaminergic receptor, in particular the human dopaminergic long D2 receptor, in particular comprising or constituted by SEQ ID NO 14,
dopaminergic receptor, in particular the human dopaminergic D3 receptor, in particular comprising or constituted by SEQ ID NO 229,
serotonergic receptor, in particular the human 5HT1α receptor, in particular comprising or constituted by SEQ ID NO 16, and
canabinoïd receptor, in particular the human CB1 receptor, in particular comprising or constituted by SEQ ID NO 230.

In one advantageous embodiment, the invention relates to a hybrid protein defined above, wherein said first and second membrane receptors are GPCR class A receptors chosen among the group comprising the chemokine receptors, and preferably chosen among the group comprising:
CXCR4 receptor, in particular the human CXCR4 receptor, in particular comprising or constituted by SEQ ID NO 18,
CCR5 receptor, in particular the human CCR5 receptor, in particular comprising or constituted by SEQ ID NO 20, and
CCR2 receptor, in particular the human CCR2 receptor, in particular comprising or constituted by SEQ ID NO 231.

In another advantageous embodiment, the invention relates a hybrid protein as defined above wherein said ion channel is Kir6.2.

In one advantageous embodiment, the invention relates to a hybrid protein defined above, wherein said ion channel is the murine or human Kir6.2 ion channel, and in particular comprises or is constituted by the amino acid sequence SEQ ID NO 2.

In another advantageous embodiment, the invention relates to a hybrid protein defined above comprising:

1—a membrane receptor sequence, in its natural configuration, fused to the N-terminus deleted sequence of Kir6.2, or
2—a membrane receptor sequence, in its natural configuration, fused to a linker sequence, said linker sequence being fused to the N-terminus deleted sequence of Kir6.2, or
3—a membrane receptor sequence, deleted in its C-terminus part, fused to the N-terminus deleted sequence of Kir6.2, or
4—a membrane receptor sequence, deleted in its C-terminus part, fused to a linker sequence, said linker sequence being fused to the N-terminus deleted sequence of Kir6.2, or
5—a membrane receptor sequence, having an addition at the C-terminus of an additional sequence from a second membrane receptor, fused to the N-terminus deleted sequence of Kir6.2, or
6—a membrane receptor sequence, having an addition at the C-terminus of an additional sequence from a second membrane receptor, fused to a linker sequence, said linker sequence being fused to the N-terminus deleted sequence of Kir6.2, or
7—a membrane receptor sequence, having an substitution at the C-terminus with a substitution sequence from a second membrane receptor, fused to the N-terminus deleted sequence of Kir6.2, or
8—a membrane receptor sequence, having an substitution at the C-terminus with a substitution sequence from a second membrane receptor, fused to the N-terminus deleted sequence of Kir6.2, fused to a linker sequence, said linker sequence being fused to the N-terminus deleted sequence of Kir6.2.

In one advantageous embodiment, the invention relates to a hybrid defined above, wherein the Kir6.2 ion channel sequence is deleted in the C-terminus part, in particular deleted from 1 to 36 of its 36 last amino acids at the C-terminus, and in particular comprises or is constituted by the amino acid sequence SEQ ID NO 4.

In one advantageous embodiment, the invention relates to a hybrid protein defined above, wherein the sequence of the Kir6.2 ion channel sequence contains an insertion of 11 amino acids, preferably contiguous, in the external loop of said Kir6.2, and in particular comprises or is constituted by SEQ ID NO 6 or by SEQ ID NO 8.

In one advantageous embodiment, the invention relates to a hybrid protein defined above, wherein said hybrid protein is chosen among the group consisting in SEQ ID NO 2q, q varying from 15 to 70 and from 99 to 102.

In one advantageous embodiment, the invention relates to a hybrid protein defined above, wherein said hybrid protein is chosen among the group consisting in SEQ ID NO 22, SEQ ID NO 24, SEQ ID NO 32, SEQ ID NO 40, SEQ ID NO 48, SEQ ID NO 56, SEQ ID NO 64, SEQ ID NO 72, SEQ ID NO 80, SEQ ID NO 88 and SEQ ID NO 94.

In one advantageous embodiment, the invention relates to a hybrid protein defined above, wherein said hybrid protein is chosen among the group consisting in SEQ ID NO 2q, q varying from 71 to 78 and SEQ ID NO 206.

In one advantageous embodiment, the invention relates to a hybrid protein defined above, wherein said hybrid protein is chosen among the group consisting in SEQ ID NO 142, SEQ ID NO 144, SEQ ID NO 146, SEQ ID NO 148, SEQ ID NO 150, SEQ ID NO 152, SEQ ID NO 154, SEQ ID NO 156 and SEQ ID NO 206.

In one advantageous embodiment, the invention relates to a hybrid protein defined above, said hybrid protein being inserted in a membrane, preferably a membrane comprising lipids.

In one advantageous embodiment, the invention relates to a nucleic acid molecule coding for a hybrid protein defined above.

In one advantageous embodiment, the invention relates to a nucleic acid molecule defined above, wherein said nucleic molecule have a nucleic acid sequence chosen among the group consisting SEQ ID 2q-1, q varying from 15 to 78 from 99 to 103.

In one advantageous embodiment, the invention relates to a vector comprising or constituted by a nucleic acid molecule defined above, and comprising elements allowing the expression of said nucleic acid molecule in host cells.

In one advantageous embodiment, the invention relates to a vector defined above, wherein said host cells are chosen among bacteria, yeast, mammal cells, insect cells or amphibian oocytes.

The invention also relates to a method for in vitro diagnosis, in a biological sample of a subject, of a pathology associated with the presence or absence or the variation of amount of a molecule modifying the receptor activity of a first membrane in its natural configuration,
said presence or absence or variation of amount of said molecule being assessed with respect to the presence or absence or the given amount of said molecule, in a sample isolated from an healthy subject, comprising:
   contacting said hybrid protein, preferably immobilized in a support, with a biological sample, said biological sample being liable to contain molecule being able to selectively interact with first membrane receptor part of said hybrid protein,
   measuring the ion current generated by the ion channel part of the said hybrid protein,
   comparing said current generated with the current generated with by said hybrid protein contacted with control sample, said control sample corresponding to sample either not containing said molecule, or containing a given amount of said molecule,
   determining, from the previous steps, if the subject is afflicted by said pathology,
said hybrid protein comprising the sequence of a first membrane receptor fused at its C-terminus to the N-terminus of a ion channel, and possibly containing a linker between the C-terminus of said first membrane receptor and the N-terminus part of said ion channel, said linker being absent in the natural configuration of said first membrane receptor and said ion channel,
said ion channel sequence being deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably being deleted from 1 to 49 amino acids at the N-terminus part of said Kir ion channel, or being deleted from 1 to 435 amino acids at the N-terminus of said Kv ion channel, possibly containing a tag sequence,
said first membrane receptor being liable to present in its extracellular domain a mutation allowing the specific interaction with a ligand different from the ligand that interact with the first membrane receptor in its natural configuration,
said first membrane receptor being liable to present in its cytoplasmic tail, said cytoplasmic tail being a sequence delimited by the last amino acids after the transmembrane domain to the last amino acid of said first membrane receptor,
   a deletion of a number of amino acids ranging from 1 to the total number of amino acids of the region delimited by the cytoplasmic tail, preferably of a number of amino acids ranging from 1 to 100, preferably of a number of amino acids ranging from 1 to 70, preferably of a number of amino acids ranging from 1 to 20 preferably of a number of amino acids ranging from 1 to 15, more preferably of a number of amino acids ranging from 1 to 10, amino acids at the C-terminus, provided that said deletion does not affect the transmembrane amino acid sequence of said membrane receptor and/or
   an addition, in particular after the last amino acid at the C-terminus of said first membrane receptor, of an additional sequence of a number of amino acids ranging from 1 to the total number of amino acids of the region delimited by the cytoplasmic tail, preferably of a number of amino acids ranging from 1 to 100 amino acids, preferably of a number of amino acids ranging from 1 to 70, preferably of a number of amino acids ranging from 1 to 20 amino acids, preferably of a number of amino acids ranging from 1 to 15 amino acids, more preferably of a number of amino acids ranging from 1 to 10 amino acids, preferably contiguous, originating from a second membrane receptor different from said first membrane receptor, preferably said additional sequence corresponding to the C-terminus of said second membrane receptor, and/or
   a substitution of a number of amino acids ranging from 1 to the total number of amino acids of the region delimited by the cytoplasmic tail, preferably a number of amino acids ranging from 1 to 20 amino acids, preferably a number of amino acids ranging from 1 to 15 amino acids, more preferably a number of amino acids ranging from 1 to 10 amino acids with an substitute sequence of a number of amino acids ranging from 1 to the total number of amino acids of the region delimited by the cytoplasmic tail, preferably a number of amino acids ranging from 1 to 20 amino acids, preferably a number of amino acids ranging from 1 to 15 amino acids, more preferably a number of amino acids ranging from 1 to 10 amino acids from a second membrane receptor different from said first membrane receptor,
said hybrid protein being such that said ion channel retains the property of electrical current generation of said ion channel in its natural configuration, and that said first membrane receptor retains the ability to interact with the ligand of said first membrane receptor in its natural configuration.

The invention also relates to a method for in vitro diagnosis, in a biological sample of a subject, of a pathology associated with the presence or absence or the variation of amount of a molecule modifying the receptor activity of a first membrane in its natural configuration,
said presence or absence or variation of amount of said molecule being assessed with respect to the presence or absence or the given amount of said molecule, in a sample isolated from an healthy subject, comprising:
   contacting said hybrid protein, preferably immobilized in a support, with a biological sample, said biological sample being liable to contain molecule being able to selectively interact with first membrane receptor part of said hybrid protein,
   measuring the ion current generated by the ion channel part of the said hybrid protein,
   comparing said current generated with the current generated with by said hybrid protein contacted with control sample, said control sample corresponding to sample either not containing said molecule, or containing a given amount of said molecule,
   determining, from the previous steps, if the subject is afflicted by said pathology,
said hybrid protein comprising the sequence of a first membrane receptor fused at its C-terminus to the N-terminus of a ion channel, and possibly containing a linker between the C-terminus of said first membrane receptor and the N-terminus part of said ion channel, said linker being absent in the natural configuration of said first membrane receptor and said ion channel,
said ion channel sequence being being deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably being deleted from 1 to 49 amino acids at the N-terminus part of said Kir ion channel, or being deleted from 1 to 435 amino acids at the N-terminus of said Kv ion channel, possibly containing a tag sequence,
said first membrane receptor being liable to present in the 70 amino acids in its C-terminus part
   a) of a number of amino acids ranging from 1 to 100, preferably from 1 to 20, preferably of a number of amino acids ranging from 1 to 15, more preferably of a number of amino acids ranging from 1 to 10, amino acids at the C-terminus, and/or
   b) an addition, in particular after the last amino acid at the C-terminus of said first membrane receptor, of an additional sequence of a number of amino acids ranging from 1 to 100, preferably from 1 to 20 amino acids, preferably of a number of amino acids ranging from 1 to 15 amino acids, more preferably of a number of amino acids ranging from 1 to 10 amino acids, preferably contiguous, originating from a second membrane receptor different from said first membrane receptor, preferably said additional sequence corresponding to the C-terminus of said second membrane receptor, and/or
   c) a substitution of a number of amino acids ranging from 1 to 20 amino acids, preferably a number of amino acids ranging from 1 to 15 amino acids, more preferably a number of amino acids ranging from 1 to 10 amino acids with an substitute sequence of a number of amino acids ranging from 1 to 20 amino acids, preferably a number of amino acids ranging from 1 to 15 amino acids, more preferably a number of amino acids ranging from 1 to 10 amino acids from a second membrane receptor different from said first membrane receptor,
said hybrid protein being such that said ion channel retains the property of electrical current generation of said ion channel in its natural configuration, and that said first membrane receptor retains the ability to interact with the ligand of said first membrane receptor in its natural configuration.

In one advantageous embodiment, the invention relates to a method for in vitro diagnosis, in a biological sample of a subject, of pathologies associated with the presence or absence or the variation of amount of a molecule modifying the receptor activity of a first membrane receptor in its natural configuration,

said presence or absence or variation of amount of said molecule being assessed with respect to the presence or absence or the given amount of said molecule, in a sample isolated from an healthy subject, comprising:
contacting said hybrid protein, preferably said hybrid protein being a hybrid protein according to anyone of claims 9 to 16, preferably immobilized in a support, with a biological sample, said biological sample being liable to contain molecule being able to selectively interact with first membrane receptor part of said hybrid protein,
measuring the current generated by the ion channel part of the said hybrid protein, preferably measured by appropriate means of electrophysiology, or reconstitution in artificial lipid bilayers, or any techniques designed to measure ion flux through potassium channels,
comparing said current generated with the current generated with by said hybrid protein contacted with control sample, said control sample corresponding to sample either not containing said molecule, or containing a given amount of said molecule,
determining, from the previous steps, if the subject is afflicted by said pathologies,
   said hybrid protein comprising or consisting in
a. the sequence of a first membrane receptor, said first membrane receptor belonging to the G-protein coupled receptors (GPCR) class A family, covalently fused at its C-terminus to
b. the N-terminus sequence of an ion channel, said ion channel belonging to the potassium channel families selected from the inwardly rectifying potassium channels (Kir) family and the voltage-dependent potassium channels (Kᵥ) family,
   said ion channel sequence being deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel,
   said ion channel possibly containing a tag sequence,
   said first membrane receptor being liable to present in its cytoplasmic tail, said cytoplasmic tail being a sequence delimited by the first amino acid after the last amino acid of the transmembrane helix and the last amino acid of said first membrane receptor,
a deletion of a number of amino acids ranging from 1 to the total number of amino acids of the region delimited by the cytoplasmic tail, and/or
an addition, of an additional sequence of a number of amino acids ranging from 1 to the total number of amino acids of the region delimited by the cytoplasmic tail, originating from a second membrane receptor different from said first membrane receptor, preferably said additional sequence corresponding to the C-terminus of said second membrane receptor and/or
a substitution of a number of amino acids ranging from 1 to the total number of amino acids of the region delimited by the cytoplasmic tail, with an substitute sequence of a number of amino acids ranging from 1 to the total number of amino acids of the region delimited by the cytoplasmic tail from a second membrane receptor different from said first membrane receptor
   said hybrid protein being such that said ion channel retains the property of electrical current generation of said ion channel in its natural configuration, and that said first membrane receptor retains the ability to interact with the ligand of said first membrane receptor in its natural configuration.

An advantageous embodiment of the invention relates to a method for in vitro diagnosis as defined above, wherein said hybrid protein comprises or consists in amino acids sequences chosen among SEQ ID NO 2q, q varying from 15 to 78 and from 99 to 103.

In the method of the invention, biological sample refers to a sample obtained from an organism or from components (e.g., cells) of an organism. The sample may be one of any biological tissue. Frequently the sample could be a “clinical sample” which is a sample derived from a patient. Such samples include, but are not limited to blood, serum, urine and lymph sample. Biological samples may also include sections of tissues such as frozen sections taken for histological purposes.

According to the invention, “molecule modifying the receptor activity” corresponds to any molecule, chemical or biological, liable to interact with said membrane receptor in its natural configuration, and as a result of this interaction modifying the natural function of said membrane receptor. For example, if a membrane receptor is implicated in the detoxification of cells, said molecule modifying the receptor activity can either inhibit or reduce this function, or enhance the function of detoxification. In this case it will be easy to define molecule with inhibiting activity as an antagonist, and molecule with enhancing activity as agonist.

In a particular aspect of the invention, the molecule modifying the receptor activity can be a natural molecule interacting with said membrane receptor, i.e. its ligand, which has been modified by one or more mutations in the corresponding gene. These mutations can have the effect to, enhance the presence of said ligand or reduce the expression of said ligand. An alternative is that these mutations do not modify the quantity of ligand but its quality, in term of functional interaction and modulation of receptor activity.

In this case, the “modified ligand” is able to interact with its receptor, but does not present the natural function and then modify the receptor activity.

Then the invention also relates to a method for in vitro diagnosis defined above, wherein pathology is associated with the presence a modified ligand molecule modifying the receptor activity of a first membrane in its natural configuration/According to the invention, the “determination of the presence” indicates that if molecule modifying the receptor activity can be detected in a biological sample, said molecule modifying the receptor activity is considered as present in the biological sample. On the contrary, if said molecule modifying the receptor activity can not be detected by the method of the invention, the molecule modifying the receptor activity is considered as absent from the biological sample.

According to the invention, the “variation of amount” molecule modifying the receptor activity means that the quantity of said molecule modifying the receptor activity is measured. The value associated to the measure of the quantity of molecule modifying the receptor activity is compared at least with a control, preferably with two control samples. The value associated to the measure is null in the control negative sample, and the value associated to the measure of the quantity of molecule is positive in the control positive sample.

The control sample corresponds to a biological sample of a healthy individual, or patient, wherein said molecule modifying the receptor activity is either absent or present at a known level, said known level being defined as the standard level.

So, if the molecule modifying the receptor activity is absent of the biological sample, the value of the quantification is null. On the other hand, if the molecule is present, the value of the quantification is superior to zero.

Then, the method of the invention consists in contacting biological sample of a subject, with hybrid protein according to the invention. Contact between biological sample and hybrid protein may allow the formation of a complex between the hybrid protein and molecule modifying the receptor activity, when present.

If a complex is formed, the hybrid protein is able to generate, via the ion channel part, an electrical signal.

Then the generated current is measured using standard electrophysiological techniques commonly used in the art. Classical electrophysiology techniques involve placing electrodes into various preparations of biological tissue. The principal types of electrodes are:

1) simple solid conductors, such as discs and needles (singles or arrays),
2) tracings on printed circuit boards, and
3) hollow tubes filled with an electrolyte, such as glass pipettes. The principal preparations include 1) living organisms, 2) excised tissue (acute or cultured), 3) dissociated cells from excised tissue (acute or cultured), 4) artificially grown cells or tissues, or 5) hybrids of the above.

The commonly used techniques to detect an ionic current through one or many channels according to the invention are, but not limited to, patch clamp, microelectrode recordings, artificial lipid bilayer recordings [Hamill O P, Marty A, Neher E, Sakmann B, Sigworth F J (1981) Improved patch-clamp techniques for high-resolution current recording from cells and cell-free membrane patches. Pflügers Arch-Eur J. Physiol. 391:85-100; Ashcroft F M (2000) Studying ion channels. In: Ion channels and disease, Academic Press; Priest B T, Swensen A M, McManus O B (2007) Automated electrophysiology in drug discovery. Curr Pharm Des. 13:2325-37], detection of current-induced changes in membrane potential using voltage-sensitive dyes or measurement of ion movement using channel-permeant detectable ions [Arkhammar P, Wahl P, Gerlach B, Fremming T, Hansen J B (2004) Establishment and application of in vitro membrane potential assays in cell lines with endogenous or recombinant expression of ATP-sensitive potassium channels (Kir6.2/SUR1) using a fluorescent probe kit. J Biomol Screen. 9:382-90; Molokanova M, Savchenko A (2008) Bright future of optical assays for ion channel drug discovery. Drug Discov Today. 13:14-22; Molokanova M, Savchenko A (2008) Bright future of optical assays for ion channel drug discovery. Drug Discov Today. 13:14-22]

This generated measured current is then compared to the measured current of at least a control sample. Said control sample has a known amount of molecules modifying receptor activity, or has none.

From the comparison between the measured current and the control current, it is possible to determine whether a modifying molecule is present, or absent, or have a modified amount. And then it is possible to conclude if the patient, from whom originates the biological sample, is afflicted by a pathology associated with the presence or absence or the variation of amount of a molecule modifying the receptor activity of a first membrane in its natural configuration.

In one advantageous embodiment, the invention relates to a method for in vitro diagnosis of pathologies defined above, wherein said hybrid protein is a hybrid protein defined above.

In one advantageous embodiment, the invention relates to a method for in vitro diagnosis of pathologies defined above, where in said generated current is measured by appropriate means of electrophysiology, or reconstitution in artificial lipid bilayers, or any techniques designed to measure ion flux through potassium channels using, for instance, channel-permeant labelled ions [Molokanova M, Savchenko A (2008) Bright future of optical assays for ion channel drug discovery. Drug Discov Today. 13:14-22].

In one advantageous embodiment, the invention relates to a method for in vitro diagnosis of pathologies defined above, wherein said ligand is chosen among growth factor, chemokine and neurotransmitter.

In one advantageous embodiment, the invention relates to a method for the in vitro diagnosis of pathologies defined above, wherein said pathologies are chosen among the group consisting in diseases characterized by abnormal hormone or neurotransmitter secretion, Neural diseases, such as Parkinson disease, Depression, Diabetes, Cardiovascular diseases, such as hyper- and hypotensive diseases, virus infection, such as HIV, chronic inflammation, asthma, obesity, pain, ischemic diseases, and cancer.

The method according to the invention gives a direct and rapid method that allow the detection of variation (presence, absence or variation of amount) of hormones, cytokines, chemokines, neurotransmitters, or any other biological molecule liable to interact with a membrane receptor.

For example, concerning HIV infection, it is known that virus need the presence of CXCR4 and CCR5 chemokines co-receptor to allow the entry of virus into the target cell [Tsibris A M, Kuritzkes D R (2007) Chemokine antagonists as therapeutics: focus on HIV-1. Annu Rev Med. 58:445-59]. Then by using a hybrid protein containing as first membrane receptor CXCR4 or CCR5 receptor, it is easy to determine, in comparison with control samples, if a biological sample contain HIV viral particles.

These two examples cited above are not limitative, and only illustrate the potential application of the hybrid protein and the method using said hybrid protein in human pathologies diagnosis.

It also interesting, according to the invention to consider that poisoning pathologies can also be detected by the method described above.

Indeed, if a poisoning molecule, such as toxin, heavy metal and derivatives, cyanide etc. . . . , is able to specifically interact with a membrane receptor, the hybrid protein according to the invention comprising said membrane receptor, would allow to detect said poisoning molecule.

Therefore, the present invention gives an easy method allowing the detection of various poisoning molecule in human or animal liquid fluids.

In one advantageous embodiment, the invention relates to a method for the in vitro diagnosis of pathologies defined above, wherein said biological sample is a body fluid, such as blood, lymph, serum, urine, sweat, saliva and cerebrospinal fluid.

The invention also relates to a method for screening a compound able to modify the receptor activity of a first membrane receptor in its natural configuration,
comprising:
   contacting a compound, with a hybrid protein as defined above, preferably immobilized on a support, in presence of a ligand of said membrane receptor,
   measuring the current generated by the ion channel of the said hybrid protein,
   comparing said current generated with the current generated with by said hybrid protein contacted with an identified compound known to modify said receptor activity,
   determining, from the previous steps the effect of said compound on the activity of said first membrane receptor in its natural configuration
said hybrid protein comprising the sequence of a first membrane receptor fused at its C-terminus to the N-terminus of a ion channel, and possibly containing a linker between the C-terminus of said first membrane receptor and the N-terminus part of said ion channel, said linker being absent in the natural configuration of said first membrane receptor and said ion channel,
said ion channel sequence being being deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably being deleted from 1 to 49 amino acids at the N-terminus part of said Kir ion channel, or being deleted from 1 to 435 amino acids at the N-terminus of said Kv ion channel, possibly containing a tag sequence,
said first membrane receptor being liable to present in its extracellular domain a mutation allowing the specific interaction with a ligand different from the ligand that interact with the first membrane receptor in its natural configuration,
said first membrane receptor being liable to present in its cytoplasmic tail, said cytoplasmic tail being a sequence delimited by the last amino acids after the transmembrane domain to the last amino acid of said first membrane receptor,
   a) a deletion of a number of amino acids ranging from 1 to the total number of amino acids of the region delimited by the cytoplasmic tail, preferably of a number of amino acids ranging from 1 to 100, preferably from 1 to 20, preferably of a number of amino acids ranging from 1 to 15, more preferably of a number of amino acids ranging from 1 to 10, amino acids at the C-terminus, and/or
   b) an addition, in particular after the last amino acid at the C-terminus of said first membrane receptor, of an additional sequence of a number of amino acids ranging from 1 to the total number of amino acids of the region delimited by the cytoplasmic tail, preferably of a number of amino acids ranging from 1 to 100, preferably from 1 to 20 amino acids, preferably of a number of amino acids ranging from 1 to 15 amino acids, more preferably of a number of amino acids ranging from 1 to 10 amino acids, preferably contiguous, originating from a second membrane receptor different from said first membrane receptor, preferably said additional sequence corresponding to the C-terminus of said second membrane receptor, and/or
   c) a substitution of a number of amino acids ranging from 1 to the total number of amino acids of the region delimited by the cytoplasmic tail, preferably a number of amino acids ranging from 1 to 20 amino acids, preferably a number of amino acids ranging from 1 to 15 amino acids, more preferably a number of amino acids ranging from 1 to 10 amino acids with an substitute sequence of a number of amino acids ranging from 1 to the total number of amino acids of the region delimited by the cytoplasmic tail, preferably a number of amino acids ranging from 1 to 20 amino acids, preferably a number of amino acids ranging from 1 to 15 amino acids, more preferably a number of amino acids ranging from 1 to 10 amino acids from a second membrane receptor different from said first membrane receptor,
said hybrid protein being such that said ion channel retains the property of ionic current generation of said ion channel in its natural configuration, and that said first membrane receptor retains the ability to interact with the ligand of said first membrane receptor in its natural configuration.

The invention also relates to a method for screening a compound able to modify the activity of a first membrane receptor in its natural configuration, said compound being preferably an agonist or an antagonist,

comprising:
contacting a compound, with a hybrid protein, preferably said hybrid protein being a hybrid protein according to anyone of claims 9 to 16, preferably immobilized on a support, in presence of a ligand of said membrane receptor,
measuring the current generated by the ion channel of the said hybrid protein, preferably measured by appropriate means of electrophysiology, or reconstitution in artificial lipid bilayers, or any technique designed to directly or indirectly measure ion flux through potassium channels,
comparing said current generated with the current generated with by said hybrid protein contacted with an identified compound known to modify said receptor activity,
determining, from the previous steps the effect of said compound on the activity of said first membrane receptor in its natural configuration
   said hybrid protein comprising or consisting in
a. the sequence of a first membrane receptor, said first membrane receptor belonging to the G-protein coupled receptors (GPCR) class A family, covalently fused at its C-terminus to
b. the N-terminus sequence of an ion channel, said ion channel belonging to the potassium channel families selected from the inwardly rectifying potassium channels (Kir) family and the voltage-dependent potassium channels (Kᵥ) family,
   said ion channel sequence being deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel,
   said ion channel possibly containing a tag sequence,
   said first membrane receptor being liable to present in its cytoplasmic tail, said cytoplasmic tail being a sequence delimited by the first amino acid after the last amino acid of the transmembrane helix and the last amino acid of said first membrane receptor,
a deletion of a number of amino acids ranging from 1 to the total number of amino acids of the region delimited by the cytoplasmic tail, and/or
an addition, of an additional sequence of a number of amino acids ranging from 1 to the total number of amino acids of the region delimited by the cytoplasmic tail, originating from a second membrane receptor different from said first membrane receptor, preferably said additional sequence corresponding to the C-terminus of said second membrane receptor and/or
a substitution of a number of amino acids ranging from 1 to the total number of amino acids of the region delimited by the cytoplasmic tail, with an substitute sequence of a number of amino acids ranging from 1 to the total number of amino acids of the region delimited by the cytoplasmic tail from a second membrane receptor different from said first membrane receptor
   said hybrid protein being such that said ion channel retains the property of electrical current generation of said ion channel in its natural configuration, and that said first membrane receptor retains the ability to interact with the ligand of said first membrane receptor in its natural configuration.

An advantageous embodiment of the invention relates to a method for screening as defined above, wherein said hybrid protein comprises or consists in amino acids sequences chosen among SEQ ID NO 2q, q varying from 15 to 78 and from 99 to 103.

The invention also relates to a method for screening a compound able to modify the receptor activity of a first membrane receptor in its natural configuration,
comprising:
   contacting a compound, with a hybrid protein, preferably immobilized on a support, in presence of a ligand of said membrane receptor,
   measuring the current generated by the ion channel of the said hybrid protein,
   comparing said current generated with the current generated with by said hybrid protein contacted with an identified compound known to modify said receptor activity,
   determining, from the previous steps the effect of said compound on the activity of
said first membrane receptor in its natural configuration said hybrid protein comprising the sequence of a first membrane receptor fused at its C-terminus to the N-terminus of a ion channel, and possibly containing a linker between the C-terminus of said first membrane receptor and the N-terminus part of said ion channel, said linker being absent in the natural configuration of said first membrane receptor and said ion channel,
said ion channel sequence being being deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel, preferably being deleted from 1 to 49 amino acids at the N-terminus part of said Kir ion channel, or being deleted from 1 to 435 amino acids at the N-terminus of said Kv ion channel,
said first membrane receptor being liable to present in the 70 amino acids in its C-terminus part
   of a number of amino acids ranging from 1 to 100, preferably from 1 to 20, preferably of a number of amino acids ranging from 1 to 15, more preferably of a number of amino acids ranging from 1 to 10, amino acids at the C-terminus, and/or
   an addition, in particular after the last amino acid at the C-terminus of said first membrane receptor, of an additional sequence of a number of amino acids ranging from 1 to 100, preferably from 1 to 20 amino acids, preferably of a number of amino acids ranging from 1 to 15 amino acids, more preferably of a number of amino acids ranging from 1 to 10 amino acids, preferably contiguous, originating from a second membrane receptor different from said first membrane receptor, preferably said additional sequence corresponding to the C-terminus of said second membrane receptor, and/or
   a substitution of a number of amino acids ranging from 1 to 20 amino acids, preferably a number of amino acids ranging from 1 to 15 amino acids, more preferably a number of amino acids ranging from 1 to 10 amino acids with an substitute sequence of a number of amino acids ranging from 1 to 20 amino acids, preferably a number of amino acids ranging from 1 to 15 amino acids, more preferably a number of amino acids ranging from 1 to 10 amino acids from a second membrane receptor different from said first membrane receptor,
said hybrid protein being such that said ion channel retains the property of ionic current generation of said ion channel in its natural configuration, and that said first membrane receptor retains the ability to interact with the ligand of said first membrane receptor in its natural configuration.

In one advantageous embodiment, the invention relates to a method for screening compound defined above, wherein said hybrid protein is a hybrid protein defined above.

In one advantageous embodiment, the invention relates to a method for screening compound defined above, wherein said known compound is an agonist or an antagonist of the activity of said first membrane receptor in its natural configuration.

In one advantageous embodiment, the invention relates to a method defined above, wherein said hybrid protein is immobilized on a support chosen among the group consisting in artificial membrane, natural or artificial membrane containing lipids, and chip comprising artificial membrane, or natural or artificial membrane containing lipids, or incorporated in the membrane of cells or liposomes.

The present invention will be better understood using the following examples 1 to 9 that are given only by way of illustration, and are non-limitative, as well as using the attached FIGS. 1 to 16 in which:

FIG. 1 schematically represents the hybrid proteins of the invention. Most cases are illustrated, i.e. the case of hybrid protein containing membrane receptor in its natural configuration, the case of hybrid protein containing membrane receptor deleted of amino acids in its C-terminus, the case of hybrid protein containing membrane receptor with a substitution of the C-terminus part of first membrane receptor with the C-terminus part of second membrane receptor, with or without linker sequence.
indicates the membrane receptor amino acids liable to be modified; indicates the ion channel amino acids liable to be modified; N—represent N-terminus and C—represents C-terminus; Linker indicates the presence of a linker sequence.

FIG. 2 schematically represents Kir6.2 protein inserted in plasma membrane. Out designates the extracellular compartment. Arrow indicates the position of external loop where tag is inserted.

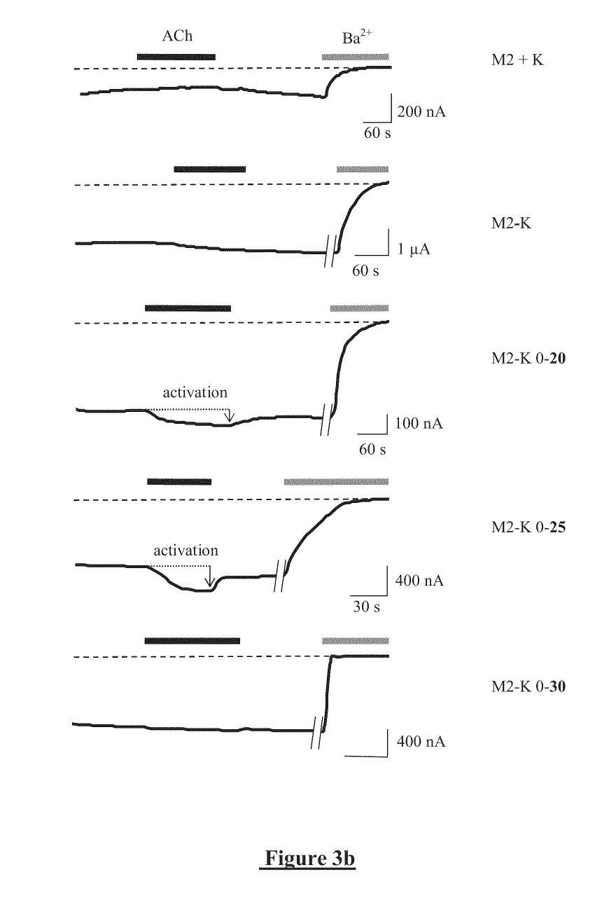

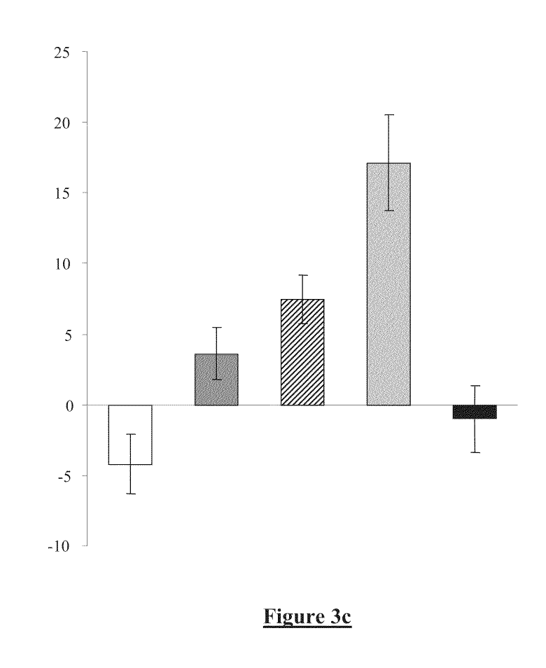

FIG. 3a represents design and basic properties of hybrids proteins comprising M2 receptor and Kir6.2 ion channel. The sequences of the region linking M2 C-ter and Kir6.2 N-ter for some experimental constructs are represented. The numbering indicated under the sequences corresponds to the numbering of amino acids of human M2 and mouse Kir6.2. The name of each construct is indicated at left. GGGGGG represents a hexa-glycine linker; —

represents a single peptide bond.

M2+K indicates that M2 and Kir6.2 are not fused and are in their natural configuration.

M2−K indicates that M2 receptor, in its natural configuration, is fused to Kir6.2, in its natural configuration.

M2−K 0-20, M2−K 0-25 and M2−K 0-30 represents hybrid protein comprising M2 receptor, in its natural configuration, respectively fused to Kir6.2 deleted of its 20 first amino acids, 25 first amino acids, or 30 first amino acids.

FIG. 3b shows representative two-electrode voltage clamp (TEVC) recordings from xenopus oocytes expressing M2+K, M2−K 0-25, M2−K 0-20 or M2−K 0-30 as indicated at right. Bath contained 150 mM K⁺ and potential was set at −50 mV so that K⁺ currents are inward and represented by convention as negative (dashed line shows 0 current level). The M2 agonist acetylcholine (ACh) was applied at 5 μM (black bar) and caused an increase in current for constructs M2−K 0-20 and M2−K 0-25. 3 mM Baryum (Ba²⁺) is used as a K channel blocker to confirm that currents arose from K channels.

FIG. 3c represents the average agonist-induced variation in currents for the different constructs obtained during experiments as in FIG. 3b. Y-axis represents the percent change in current induced by acetylcholine [5 μM]. White column represents M2+K proteins; dark grey column represents M2−K hybrid protein; stripped column represents M2−K 0-20 hybrid protein; light grey column represents M2−K 0-25 hybrid protein; and black column represents M2−K 0-30 hybrid protein. Data is mean±standard error. N was >10.

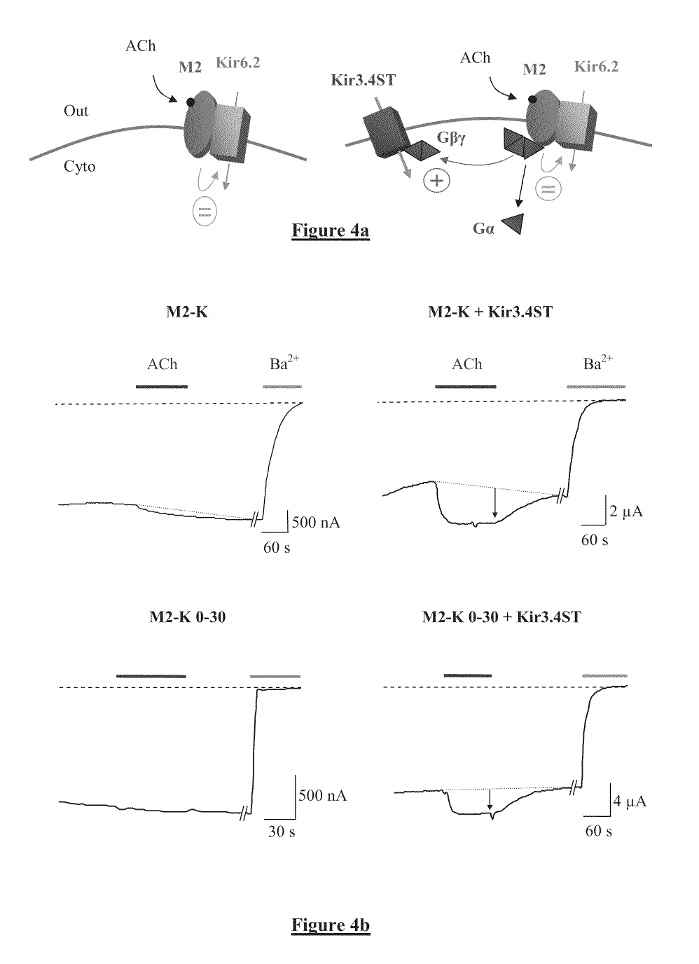

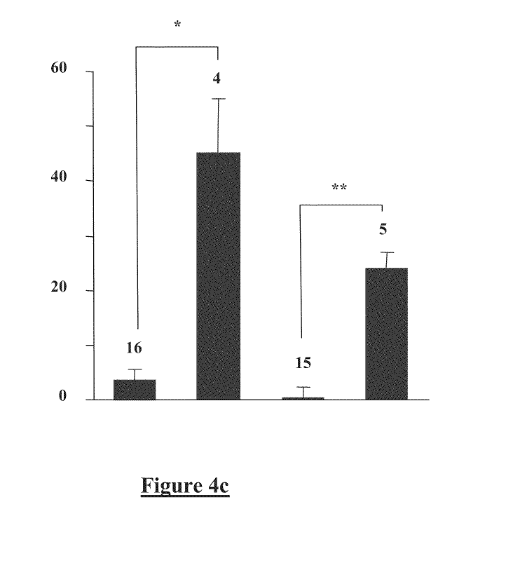

FIG. 4a schematically represents, on the left, the hybrid protein M2−Kir6.2 inserted in the plasma membrane, and on the right, the intracellular connection between M2 receptor comprised in the M2-Kir6.2 hybrid protein and the natural G-protein-activated ion channel Kir3.4ST. Black ball represents acetylcholine (ACh); triangle represents hetero-trimeric G proteins.

FIG. 4b shows representative two-electrode voltage clamp (TEVC) recordings from xenopus oocytes expressing M2−K (upper left), M2−K⁺ Kir3.4ST (upper right), M2−K 0-30 (lower left) and M2−K⁺ Kir3.4ST (lower right). Bath contained 150 mM K⁺ and potential was set at −50 mV so that K⁺ currents are inward and represented by convention as negative (dashed line shows the baseline of baryum-sensitive current). The M2 agonist acetylcholine (ACh) was applied at 5 μM (black bar) and caused an increase in current for constructs M2−K⁺ Kir3.4ST and M2−K 0-30+Kir3.4ST. 3 mM Baryum (Ba²⁺) is used as a K channel blocker to confirm that currents arose from K channels.

FIG. 4c is a graph representing the average agonist-induced variation in currents for the different constructs obtained during experiments as in FIG. 4b. The Y-axis represents the percentage of change induced by 5 μM of ACh. First column represents M2−K results, second column represents M2-K+Kir3.4ST results, third column M2−K 0-30 results and fourth column represents M2−K 0-30+Kir3.4ST results. Results are means±standard error. * and ** represent significant differences.

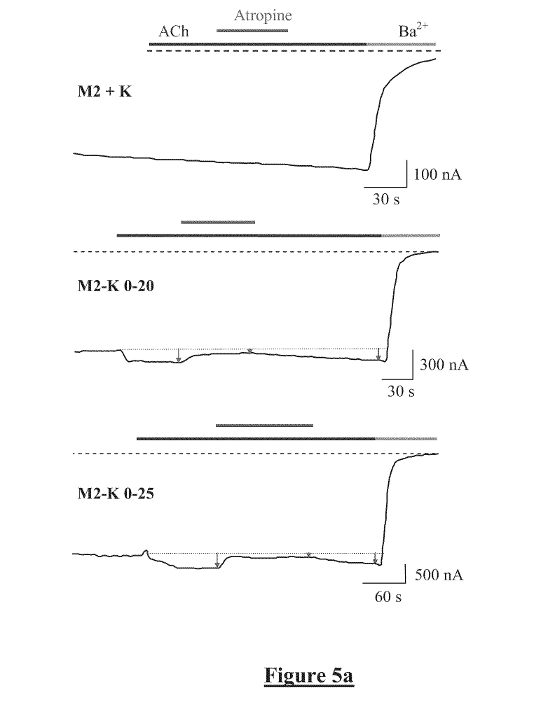

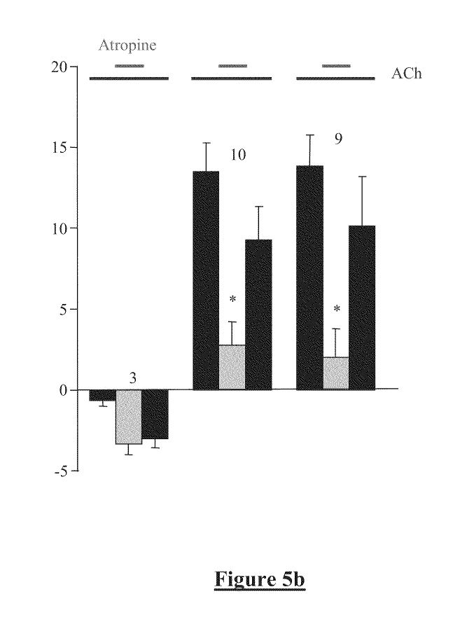

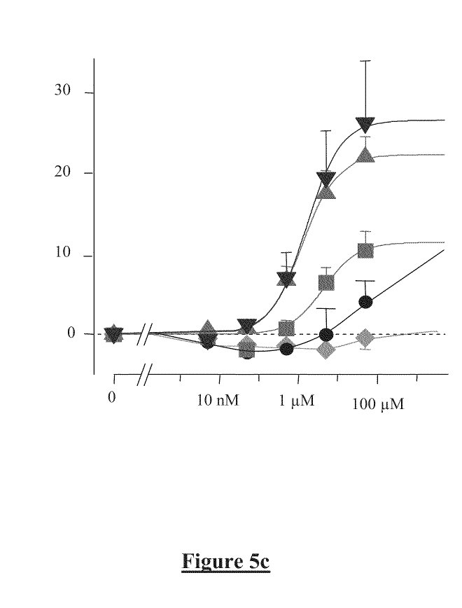

FIG. 5a shows representative two-electrode voltage clamp recordings from xenopus oocytes expressing M2+K, M2−K 0-20 or M2−K 0-25 as indicated. The M2 agonist ACh was applied at 5 μM (black bar) and caused an increase in current for constructs M2−K 0-20 and M2−K 0-25. Also the M2 antagonist atropine was supplied at 1 μM (upper bar) and inhibits the ACh-induced current of constructs M2−K 0-20 and M2−K 0-25. 3 mM Baryum (Ba²⁺) is used as a K channel blocker to confirm that currents arose from K⁺ channels.

FIG. 5b is a graph representing the compilation of data obtained in many experiments as in FIG. 5a.

The Y-axis represents the percentage of change in current.

Black column represents current variation in presence of ACh, and grey column represent the current variation in presence of ACh+Atropine. Results are means±standard error. * represents significant differences. First group of column represents M2+K, second group of column represents M2−K 0+20, and third group of column represents M2−K 0-25

FIG. 5c is a graph indicating the % change of the current induced by different Carbachole (CCh) concentrations, a M2 activator. The Y-axis represents the percentage induced by CCh, and X-axis represents the concentration in CCh (logarithm scale).

Ball represents data of M2+K, Square represents data of M2−K (with 4.3 μM), inverted triangle represents data of M2−K 0-20, triangle represents data of M2−K 0-25 and lozenge represents data of M2−K 0-30.

FIG. 6a shows representative two-electrode voltage clamp recordings from xenopus oocytes expressing M2+Kir3.4ST, M2+Kir3.4ST, M2−K 0-25 or M2−K 0-25+Kir3.4ST as indicated. The M2 agonist ACh was applied at 5 μM (black bar). Also, Pertussis Toxin (PTX) was co-expressed with membrane proteins where indicated. 3 mM Baryum (Ba²⁺) is used as a K channel blocker to confirm that currents arose from K channels.

FIG. 6b is a graph indicating the % change induced by ACh concentrations. Y-axis represents the percentage of change induced by ACh. First column represents M2+Kir3.4ST, second column represents M2+Kir3.4ST+PTX, third column represents M2−K 0-25 and fourth column represents M2−K 0-25+PTX. Results are means±standard error. * and ** represent significant differences.

FIG. 7 shows representative voltage clamp recordings from xenopus oocytes expressing K or M2 M2−K 0-25 as indicated, in outside out patches excised from oocytes membranes. The M2 agonist ACh was applied at 5 μM (black bar). Also, Pertussis Toxin (PTX) was co-expressed with membrane proteins where indicated. 3 mM Baryum (Ba²⁺) is used as a K channel blocker to confirm that currents arose from K channels.

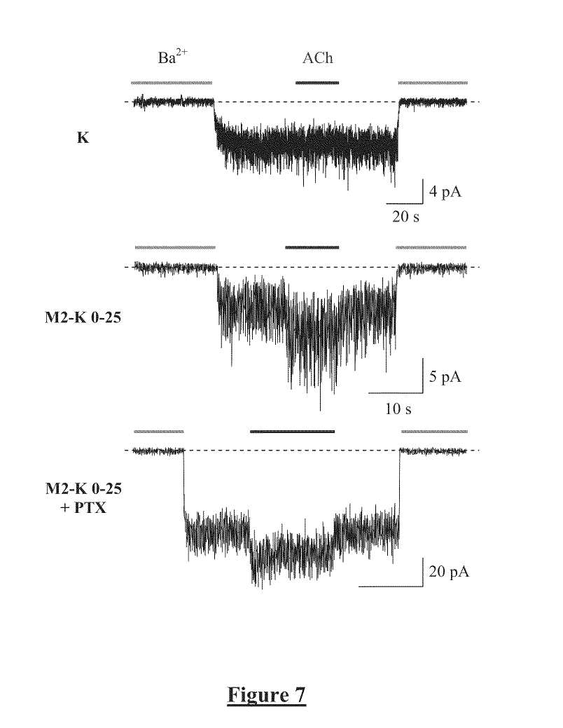

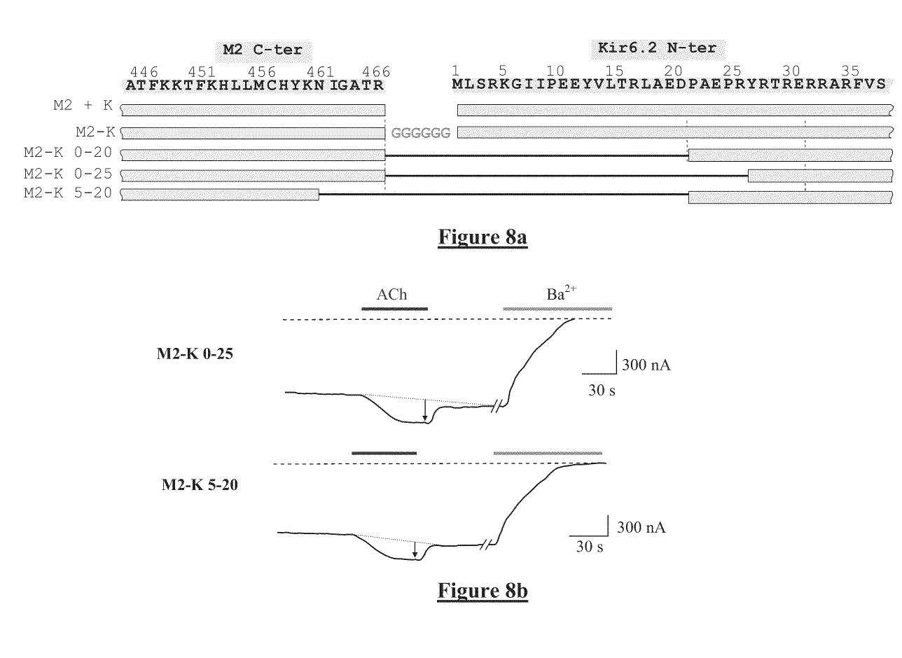

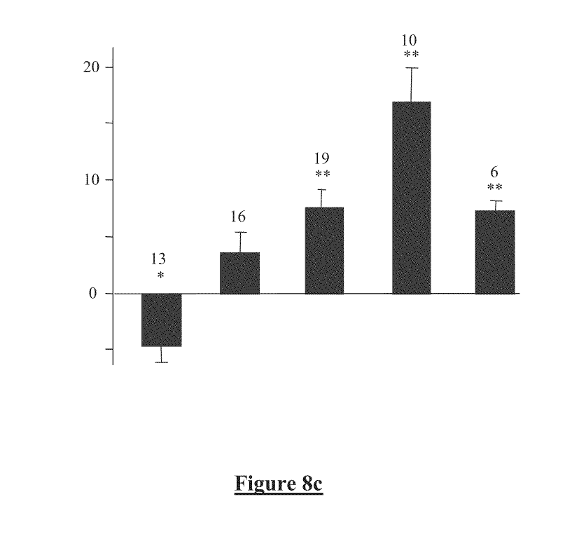

FIG. 8a represents design and basic properties of hybrid proteins comprising M2 receptor and Kir6.2 ion channel. The sequences of the region linking M2 C-ter and Kir6.2 N-ter for experimental constructs are represented. The numbering indicated under the sequences corresponds to the numbering of amino acids of human M2 and mouse Kir6.2. The name of each construct is indicated at left. GGGGGGG represents a hexa-glycine linker; —

represents a single peptide bond.

M2+K indicates that M2 and Kir6.2 are not fused and are in their natural configuration.

M2−K indicates that M2 receptor, in its natural configuration, is fused to Kir6.2, in its natural configuration.

M2−K 5-20 indicates that M2 receptor deleted of its 5 last amino acids is fused to Kir6.2, deleted of its 20 first amino acids.

FIG. 8b shows representative two-electrode voltage clamp recordings from xenopus oocytes expressing M2−K 0-25 or M2−K 5-20 as indicated. The M2 agonist ACh was applied at 5 μM (black bar) and caused an increase in current for constructs M2−K 0-25 or M2−K 5-20. 3 mM Baryum (Ba²⁺) is used as a K channel blocker to confirm that currents arose from K channels.

FIG. 8c is a graph indicating the % change of current induced by ACh concentrations. Y-axis represents the percentage of change induced by ACh. First column represents M2+K, second column represents M2−K, third column represents M2−K 0-20, fourth column represents M2−K 0-25 and fifth column represents M2−K 5-20. Results are means±standard error. * and ** represent significant differences.

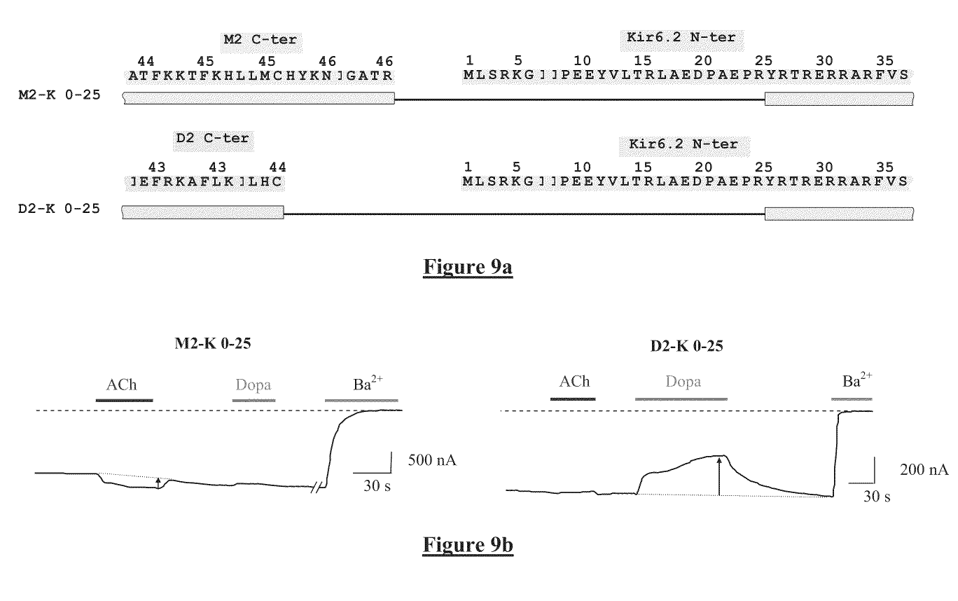

FIG. 9a represents design and basic properties of hybrids proteins comprising M2 receptor or D2 receptor, and Kir6.2 ion channel. The sequences of the region linking M2/D2 C-ter and Kir6.2 N-ter for experimental constructs are represented. The numbering indicated under the sequences corresponds to the numbering of amino acids of human M2 or D2 and mouse Kir6.2. The name of each construct is indicated at left. GGGGGG represents a hexa-glycine linker; represents a single peptide bond. —

D2-K 0-25 indicates that D2 receptor in its natural configuration is fused to Kir6.2, deleted of its 25 first amino acids.

FIG. 9b shows representative two-electrode voltage clamp recordings from xenopus oocytes expressing M2−K 0-25 or D2-K 0-20 as indicated. The M2 agonist ACh was applied at 5 μM (black bar) and caused an increase in current for constructs M2−K 0-25. Further, the D2 agonist Dopamine (Dopa) was applied at 5 μM (black bar) and caused an increase in current for constructs D2-K 0-25. 3 mM Baryum (Ba²⁺) is used as a K channel blocker to confirm that currents arose from K channels.

FIG. 9c is a graph indicating the % change of current induced by Dopamine concentrations. Y-axis represents the percentage of change induced by Dopamine. The first column represents D2+K, the second column represents M2−K 0-25 and the third column represents D2-K 0-25. Results are means±standard error. * represents significant differences.

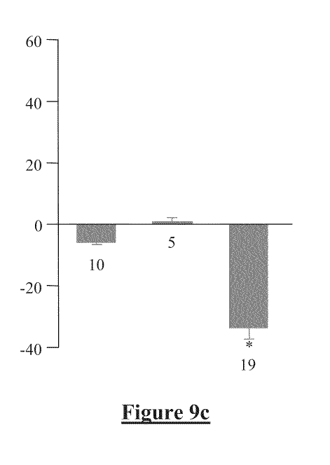

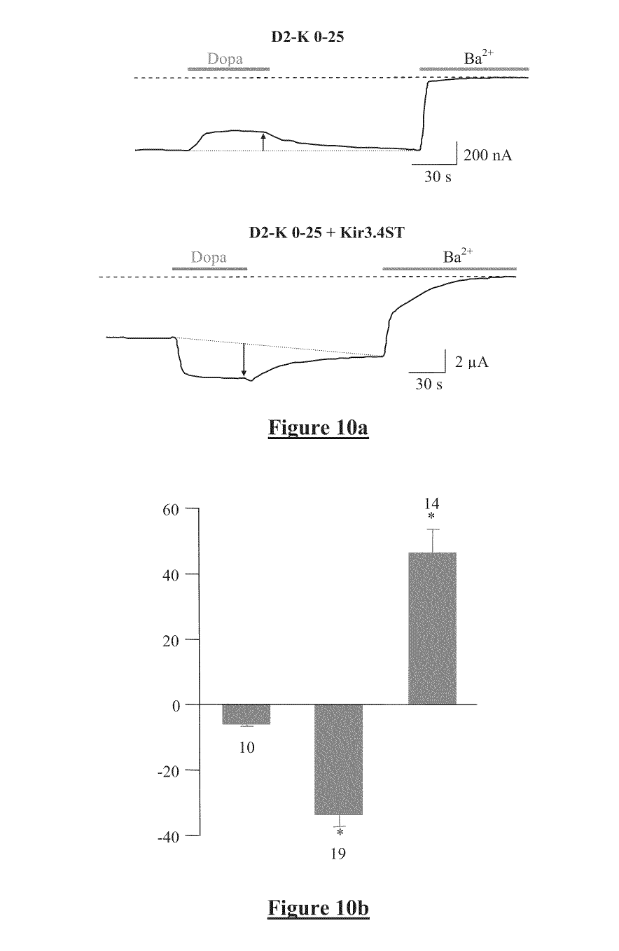

FIG. 10a shows representative two-electrode voltage clamp recordings from xenopus oocytes expressing D2-K 0-25 or D2-K 0-25+Kir3.4ST as indicated. The D2 agonist Dopamine was applied at 5 μM (black bar). 3 mM Baryum (Ba²⁺) is used as a K channel blocker to confirm that currents arose from K channels.

FIG. 10b is a graph indicating the % change of current induced by Dopamine concentrations. Y-axis represents the percentage of change induced by Dopamine. The first column represents D2+K, the second column represents D2-K 0-25 and the third column represents D2-K 0-25+Kir3.4ST. Results are means±standard error. * represents significant differences.

FIG. 11 shows representative two-electrode voltage clamp recordings from xenopus oocytes expressing D2-K 0-25 as indicated. The D2 agonist Quinpirole (Quin) was applied at 5 μM. During Quin treatment, D2 antagonist Sulpiride was supplied at 5 μM. 3 mM Baryum (Ba²⁺) is used as a K channel blocker to confirm that currents arose from K channels

FIG. 12 shows representative two-electrode voltage clamp (TEVC) recordings from xenopus oocytes expressing β2-K Δ62-25 Ha as indicated. Bath contained 150 mM K⁺ and potential was set at −50 mV so that K⁺ currents are inward and represented by convention as negative (dashed line shows 0 current level). The β2 agonist Isoproterenol (Iso) was applied at 0.5 μM (black bar) and caused an increase in current. 3 mM Baryum (Ba²⁺) is used as a K channel blocker to confirm that currents arose from K channels.

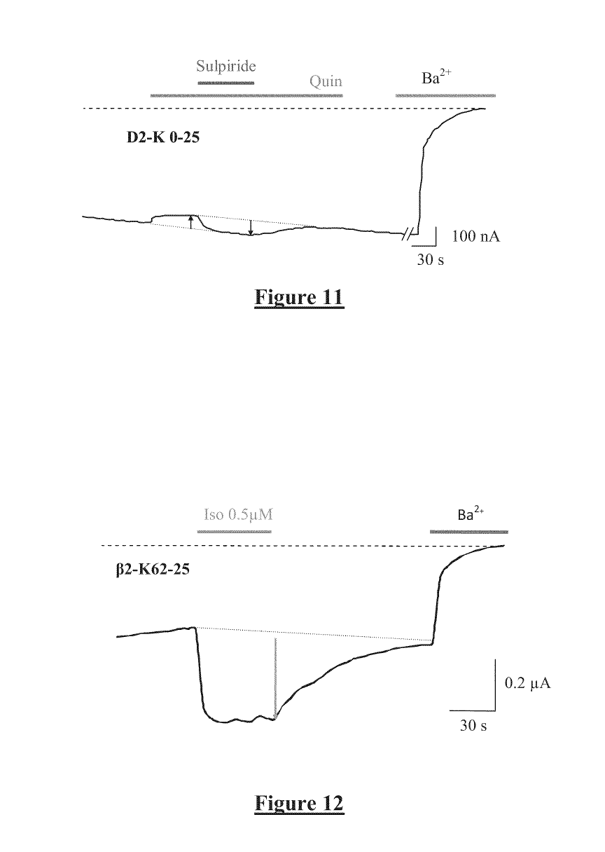

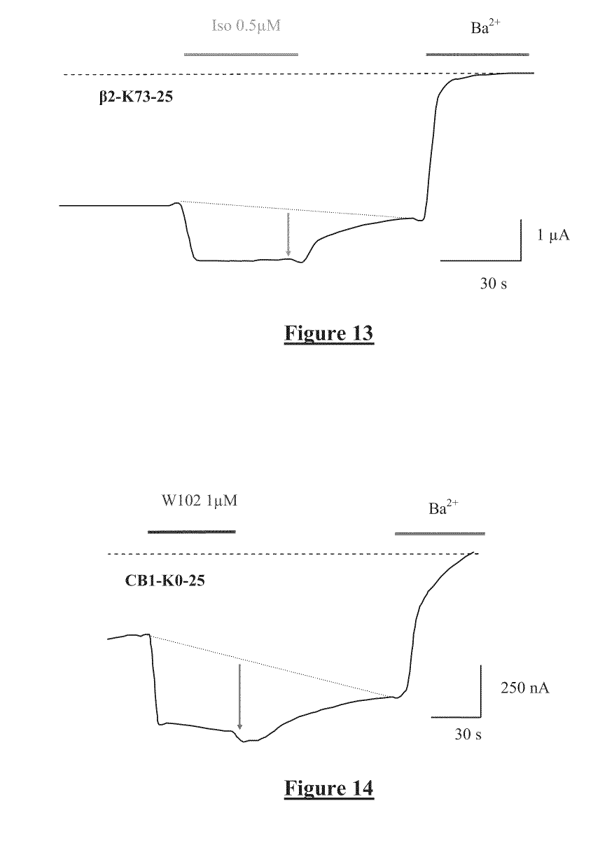

FIG. 13 shows representative two-electrode voltage clamp (TEVC) recordings from xenopus oocytes expressing β2-K Δ73-25 Ha as indicated. Bath contained 150 mM K⁺ and potential was set at −50 mV so that K⁺ currents are inward and represented by convention as negative (dashed line shows 0 current level). The β2 agonist Isoproterenol (Iso) was applied at 0.5 μM (black bar) and caused an increase in current. 3 mM Baryum (Ba²⁺) is used as a K channel blocker to confirm that currents arose from K channels.

FIG. 14 shows representative two-electrode voltage clamp (TEVC) recordings from xenopus oocytes expressing CB1-K Δ0-25 Ha as indicated. Bath contained 150 mM K⁺ and potential was set at −50 mV so that K⁺ currents are inward and represented by convention as negative (dashed line shows 0 current level). The CB1 agonist WIN 55, 212-2 (W102) was applied at 1 μM (black bar) and caused an increase in current. 3 mM Baryum (Ba²⁺) is used as a K channel blocker to confirm that currents arose from K channels.

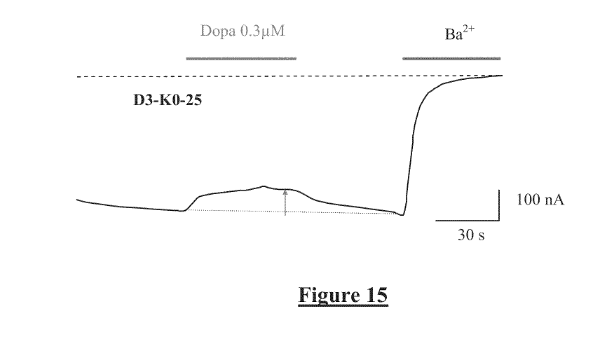

FIG. 15 shows representative two-electrode voltage clamp (TEVC) recordings from xenopus oocytes expressing D3-K Δ0-25 Ha as indicated. Bath contained 150 mM K⁺ and potential was set at −50 mV so that K⁺ currents are inward and represented by convention as negative (dashed line shows 0 current level). The D3 agonist Dopamine (Dopa) was applied at 1 μM (black bar) and caused an increase in current. 3 mM Baryum (Ba²⁺) is used as a K channel blocker to confirm that currents arose from K channels.

FIG. 16 represents the structure comparison of secondary conformation of Kir channels and Kv channels. Helices represent α-helices, arrows represents β-sheets. 1 represents the beginning of the amino acid sequence, and E represents the End of the amino acid sequence.

The structural homology between the two types of ions channels is grey boxed. In the grey box, A represents the cytoplasmic helices, inner and outer represents the two transmembrane helices and pore represent the pore helix.

FIG. 17 represents the sequence alignment of the human Kir ion channels.

### EXAMPLES

### Example 1

### Deleted Kir6.2 Ion Channel Comprised in Hybrid Protein is Able to Generate a Signal Upon Stimulation of Attached Receptor

In order to develop biosensors able to detect membrane receptor activation, the Inventors have developed hybrid proteins comprising membrane receptor fused to ion channel Kir6.2.

Kir6.2 ion channel was chosen, not only because it is part of a K_{ATP} channel, but also because it is a relatively simple, well-studied potassium (K⁺) channel that has the unique signature of being inhibited by intracellular ATP. This inhibiting property is a convenient feature that provides a straightforward means to identify the channel and control its open probability.

The first membrane receptor tested in the present invention is the muscarinic M2 receptor. This receptor is activated by the neurotransmitter acetylcholine (ACh), by the synthetic analogue Carbachol, and by the toxin Muscarine. It is blocked by atropine. M2 receptors are present in many tissues, including neurons and muscles. In heart, they mediate heart rate slowing upon vagal stimulation.

To test the efficiency of the fusion between membrane receptor and ion channel, many constructs have been constructed and in particular
M2−K
M2−K 0-20
M2−K 0-25
M2−K 0-30

The efficiency in the ability to generate an electrical signal upon ligand binding to the receptor was compared to the two non-fused proteins, i.e. in their natural configuration.

### A. Protein Engineering

M2−Kir6.2 fusion proteins were created by insertion of the human M2 coding sequence at the 5′ end of the Kir6.2 gene cloned in the pGH2 vector (derived from the pGEMHE vector optimized for protein expression from RNA in xenopus oocytes [Liman E R, Tytgat J, Hess P (1992) Subunit stoichiometry of a mammalian K⁺ channel determined by construction of multimeric cDNAs. Neuron. 9:861-71]. In a first PCR reaction, the human M2 gene was amplified with hybrid primers complementary to the M2 sequence at one extremity and the sites of insertion in pGH2-Kir6.2 at the other. The products of this reaction were gel-purified and served as primers for a second PCR with pGH2-Kir6.2 as template, yielding pGH2-M2−Kir6.2.

Using this construct as template, other constructs with shortened M2−Kir6.2 linkers were obtained by deletion of the appropriate codons with a single PCR reaction with hybrid primers flanking the deletion [Makarova O, Kamberov E, Margolis B (2000) Generation of deletion and point mutations with one primer in a single cloning step. Biotechniques. 29:970-2].

Reagents and conditions are from the QuikChange site-directed mutagenesis kit (Stratagene). Valid clones were identified by restriction enzyme profiling and verified by sequencing of the full open reading frame.

The hybrid proteins junctions are represented in FIG. 3a

The hybrid proteins were then expressed in Xenopus laevii oocytes, in order to measure their ability to generate an electrical signal upon ligand binding to the receptor.

### B. Hybrid Production and Transfection

cRNAs were produced in vitro with the T7 mMessage mMachine kit (Ambion), by using the cloned hybrid constructions described above

cRNA were purified by standard phenol/chloroform extraction, and quantified by agarose-gel electrophoresis and spectrophotometry

Xenopus oocytes defoliculated by collagenase treatment were microinjected with 50 nl of water containing one or a mixture of the following quantities of cRNA:

M2−Kir6.2 constructs, ˜5 ng;

### M2, ˜2 ng; and

### Kir6.2, ˜2 ng

Oocytes were incubated in individual wells in Barth's solution (KCl 1 mM, MgSO₄ 0.82 mM, NaCl 88 mM, NaHCO₃ 2.4 mM, CaCl₂ 0.41 mM, Ca(NO₃)₂ 0.3 mM, Hepes 16 mM, pH 7.4) supplemented with 100 U·ml⁻¹ penicillin, 100 μg ml⁻¹ streptomycin and 100 μml⁻¹ gentamycin, for at least 48 hrs at 19° C. before characterization.

Because of the inherent variability of the oocyte expression system, related experiments (e.g., with and without PTX) were alternated and performed on the same days with the same batches of oocytes.

After 48 h, electrophysiological measurement was performed in each type of injected oocytes.

### C. Electrophysiological Recordings.

Excised inside-out patch-clamp recordings were performed with symmetrical 150 mM K¹ as follows. Patch pipettes contained: 154 mM K⁺, 146 mM Cl⁻, 5 mM Mg²⁺ and 10 mM PIPES-KOH (pH 7.1). The cytoplasmic face of the patch was bathed in solutions containing 174 mM K⁺, 40 mM Cl⁻, 1 mM EGTA, 1 mM Mg²⁺, 10 mM PIPES-KOH (pH 7.1) and methanesulfonate as the remaining anion. ATP was added as specified. Membrane potential was held at −50 mV during all experiments. Application of various solutions to the patch was performed using a RSC-100 automated sewer pipes system (Bio-Logic). Pipe switching time was set at 250 ms. Data acquisition and analysis were performed using in-house software. Baseline fluctuations were removed by interactive fitting with a spline curve and subtraction of this fit with the signal. Non-linear curve fittings were performed with Origin software (OriginLab).

In the outside-out configuration, methods were identical and solutions were identical except that bath and pipette solutions were swapped and the pipette was supplemented with ATP as specified and 1 mM MgCl₂

Whole-cell currents were measured using two-electrode voltage clamp (TEVC). Microelectrodes were filled with 3 M KCl and oocytes were bathed in a solution having (in mM): 91 KCl, 1.8 CaCl₂, 1 MgCl2, 5 HEPES (pH 7.4) and 0.3 niflumic acid to block endogenous chloride current.

Unless otherwise specified, the concentration of M2 ligands was 5 μM.

Ba²⁺ (BaCl2) concentration was always 3 mM, a concentration sufficient to fully block Kir6.2 currents.

The TEVC voltage protocol consisted of 500-ms steps to −50, 0 and +50 mV—during which current was measured—repeated every 5 s, the holding potential being 0 mV.

Only the values measured at −50 mV are shown in the figures. Average values are presented as mean±s.e.m. Non-linear least-square curve fitting to the data points obtained at various concentrations of activating or inhibiting ligands was done using a standard Hill equation:

f(x)=a+b/[1+(K_{1/2}/x)^{z*h}],

where x is the concentration of ligand, a and b are scaling constants, z is either +1 for inhibition or −1 for activation, K_{1/2}, is the concentration for half-maximal effect and h is the Hill coefficient.

Ba²⁺ was used as a generic K⁺ channel blocker. Percent changes in current were calculated with respect to the baseline extrapolated from measurements of Ba²⁺-sensitive currents before and after agonist application. This method emphasizes reversibility and provides an underestimate of the true effects. For dose-response data, which were acquired by applying sequentially increasing concentrations of modulator to the same patch or oocyte, changes in current could only be measured with respect to the current before application, yielding somewhat larger values.

### D. Results

1— N-ter deleted Kir6.2 Comprising Hybrid Protein is Able to Generate an Electrical Signal Upon stimulation of attached receptor

All oocytes were stimulated with acetylcholine for times ranging from 10 seconds to 5 minutes, and the variation of current was measured as described above.

FIG. 3b represents typical time courses of −50 mV currents measured in oocytes expressing M2+K, M2−K, M2−K 0-20, M2−K 0-25 and M2−K 0-30. FIG. 3c summarizes the results of these experiments.

As expected, M2+K co-expression does not generate any variation in current when oocytes are subjected to an application of acetylcholine. This co-expression corresponds to the negative control and shows that Kir6.2 is not regulated by the presence of ACh.

Whereas hybrid protein M2−K comprising membrane receptor and ion channel in their natural configuration does not show a detectable ACh response, hybrid proteins M2−K 0-20 and M2−K 0-25 show an increase in the measured current when oocytes are stimulated with ACh. The interpretation of these results is that the receptor and the ion channel within the hybrid protein must be sufficiently close for the ligand-induced conformational change of the receptor to be transduced into a conformational change of the ion channel sufficient to alter channel gating.

As shown in FIG. 3c, ACh responses increase with longer N-ter channel deletions. However, ACh responses are abrogated by large deletions, like the deletion of 30 amino acids of the hybrid protein M2−K 0-30, probably because such deletions alter key structural secondary structures of the ion channel. Optimal responses are obtained with deletions of 20 to 25 amino acids.

### 2—Membrane Receptor in its Natural Configuration Comprised in the Hybrid Protein Activates its Normal Signalling Pathway.

In cells, M2 receptor, in its normal configuration, is able to activate Kir3.4-S143T (Kir3.4ST) potassium channels, via the activation of a heterotrimeric G protein [Vivaudou M, Chan K W Sui J L, Jan L Y, Reuveny E, Logothetis D E (1997) Probing the G-protein regulation of GIRK1 and GIRK4, the two subunits of the K-ACh channel, using functional homomeric mutants. J Biol. Chem. 272: 31553-60]. When M2 receptor is activated, it becomes capable of activating associated heterotrimeric G proteins by catalyzing the binding of GTP to Gα and the subsequent release of the G protein subunits Gα and Gβγ. Gβγ in turn can bind to the cytosolic part of the Kir3.4ST channel and activate it. Kir3.4ST can thus serve as an indicator that a receptor is functional and it was used to test whether the receptor in hybrid proteins remained functional and indistinguishable from the unaltered, unfused receptor.

FIG. 4a illustrates such experiments.

In order to test if membrane receptor in its natural configuration is still able to activate its normal signaling pathway, when it is comprised in a hybrid protein, current generation was estimated in oocytes as described above.

Xenopus oocytes were transfected with M2−K or M2−K 0-30 hybrid protein with or without Kir3.4ST ion channel. FIG. 4b represents typical TEVC recordings from oocytes expressing the specified constructs and FIG. 4c shows the mean ACh-induced changes in current obtained in these experiments (error bars represent standard error of the mean; numbers above bars indicate the number of experiments considered; stars indicate a statistically significant difference at p<0.01). In oocytes expressing hybrid proteins only, no current variation was measured, after ACh stimulation, as expected because neither M2−K nor M2−K 0-30 display ACh response as shown above. However, when Kir3.4ST was coexpressed, oocytes expressing M2−K or M2−K 0-30 hybrid proteins displayed large ACh-induced currents indicative of Kir3.4ST activation by released G-protein subunits. We may reasonably extrapolate that the receptor in other hybrid proteins M2−K 0-20 and M2−K 0-25 would behave also as an unfused receptor.

These results allow to conclude:
than the absence of agonist-induced current generated by hybrid proteins wherein ion channel part is not deleted of the first N-terminus amino acids, or is deleted too much, is due to a dysfunction of the coupling between channel and receptor rather than a dysfunction of the membrane receptor part, and,
that membrane receptor comprised in hybrid proteins of the invention retains its ability to activate normal signaling pathway and therefore is functionally, and probably structurally, indistinguishable from the membrane receptor in its physiological form.

### 3—Hybrid Proteins Produce Distinct Signals for Receptor Agonists and Antagonists

Another question was to know whether hybrid proteins can distinguish between an agonist and an antagonist of the receptor.

Functionally active hybrid proteins M2−K 0-20 and M2−K 0-25, as well as M2+K proteins, were expressed in Xenopus oocytes, and stimulated with ACh and with an antagonist of ACh, atropine.

FIG. 5a shows typical TEVC recordings from oocytes expressing the specified constructs, and FIG. 5b summarizes these experiments (error bars represent sem; numbers above bars indicate the number of experiments considered; stars indicate a statistically significant difference at p<0.01).

To test the response to another agonist, we used another M2 activator, carbachol (CCh), and we further examined the CCh dose-response relation of the hybrid protein signals. FIG. 5c shows the dose response in % of the variation of current observed in Xenopus oocytes expressing M2+K, M2−K, M2−K 0-20, M2−K 0-25 or M2−K 0-30. The results clearly demonstrate that fusion proteins able to generate a current are stimulated, in a dose-dependent manner by the M2 agonist CCh. The values of affinities indicated in parenthesis correlate well with expected values from the literature.

The invention therefore permits to directly test antagonists as well as agonists and to obtain apparent affinities (or potencies).

### 4—The Communication Between Membrane Receptor and Ion Channel Parts of Hybrid Proteins is Direct.

The results represented above indicate that
a deletion of the N-terminus first amino acids of Kir6.2 are needed to generate a signal in response to binding of ligand to the attached receptor,
membrane receptor part remains fully functional within the hybrid protein as it retains the ability to activate normal signalling pathways that are activated by the membrane receptor in its natural configuration, and
agonists and antagonists of the membrane receptor modulate in opposite ways the current generated by the channel part of the hybrid protein.

A crucial question is whether the communication between the two parts of the hybrid protein is direct, or requires the presence of accessory messengers and proteins.

a—Use of G-Protein Inhibitors

The first approach to answer the above question was to use an inhibitor of the G-proteins Gi/o activated by the M2 receptor; the catalytic S1 subunit of Pertussis toxin (PTX). PTX blocks G-protein activation via M2 by ADP-rybosylating the alpha subunits of Gi/o G-proteins. The fusion protein M2-K 0-25 was expressed in Xenopus oocytes with or without co-expressed PTX. As a control, the unmodified M2 receptor was expressed in oocytes with Kir3.4ST with or without co-expressed PTX.

FIG. 6a shows typical TEVC recordings from oocytes expressing the specified constructs, and FIG. 6b summarizes these experiments (error bars represent sem; numbers above bars indicate the number of experiments considered; single star indicates a statistically significant difference at p<0.01 while double star indicates no significant difference).

In oocytes expressing M2+Kir3.4ST, ACh induces an increase in current that reflects the G-protein mediated activation of Kir3.4ST. This effect is abolished when oocytes co-expressed PTX, in agreement with the role of G-proteins in activation of Kir3.4ST.

In oocytes expressing M2−K 0-25, ACh induces an increase in current as described above. However, co-expressed PTX does not significantly modify the ACh-induced current.

These results indicate that the effect of ACh on the hybrid protein current does not involve G-proteins and is likely to be due to a direct, physical interaction between channel and receptor.

b—Cell-Free Current Measurement

Another way to test the direct transmission of signal from M2 to Kir6.2 upon application of ACh, is to test the effect of ACh in isolated patches of membrane where no signalling pathway involving soluble second messengers can be preserved. To this end, we used the excised outside-out configuration of the patch clamp technique. FIG. 7 shows typical current recordings measured in outside-out patches excised from oocytes expressing K, M2−K 0-25 or M2−K 0-25 with PTX. The application to the extracellular face of the membrane of 5 μM ACh has no effect on Kir6.2ΔC36 (K) as expected but it causes activation of M2−K 0-25 as in whole cells. This activation was preserved in the presence of PTX ruling out a G-protein mediated effect. Therefore, communication between receptor and channel is independent of G-proteins and soluble messengers and is likely direct.

This cell-free demonstration demonstrates that hybrid proteins can function in artificial lipid bilayers.

### Example 2

### Deletion of Last Amino Acids in the C-Terminus Part of Membrane Receptor does not Modify the Ability of Hybrid Protein to Generate a Signal

The previous example has illustrated the importance of limited deletions of the first N-terminal amino acids of Kir6.2 in signal generation.

In this example, the deletion of the last C-terminal amino acids of the membrane receptor is evaluated. The previously shown hybrid proteins were compared with the hybrid protein M2−K 5-20, wherein the M2 receptor deleted of the 5 last amino acids of the C-terminus is fused to Ki6.2 deleted of the first 20 first amino acids of the N-terminus. FIG. 8a represents the junction between the two proteins.

FIG. 8b shows typical TEVC recordings from oocytes expressing the specified constructs, and FIG. 8c summarizes these experiments (error bars represent sem; numbers above bars indicate the number of experiments considered; single star indicates a statistically significant difference from 0 at p<0.05 while double stars indicate a statistically significant difference from 0 at p<0.01).

These results demonstrate that a deletion in the C-terminus of the membrane receptor comprised in the hybrid protein does not affect its ability to generate a receptor-dependent signal.

### Example 3

### Hybrid Protein Comprising Kir6.2 and D2 Dopaminergic Receptor

So far, hybrid proteins incorporating the M2 receptor have been demonstrated. To prove the general nature of the invention, a distinct receptor was used to construct hybrid protein. Because a deletion of 25 amino acids from Kir6.2 was optimal, we directly constructed the hybrid protein D2-K 0.25 described in FIG. 9a and tested the effects of the physiological agonist Dopamine (Dopa).

FIG. 9b represents typical time courses of −50 mV currents measured in oocytes expressing M2−K 0-25 or D2-K 0.25. FIG. 9c summarizes these experiments (error bars represent sem; numbers above bars indicate the number of experiments considered; single star indicates a statistically significant difference from 0 at p<0.01). M2−K 0-25 responds to ACh as shown above and is insensitive to Dopa, not known as a M2 effector. In contrast, D2-K 0-25 responds to Dopa (5 μM) and is insensitive to ACh, not known as a D2 effector. As opposed to the effect of ACh on M2−K 0-24, Dopa caused a decrease in current of D2-K 0-25. Although the signal is opposite, it remains clearly detectable. This inhibition of D2-K 0-25 was dependent on the dopamine concentration (K_{1/2}=73 nM, n=6; Data not shown)

Like M2, the fused D2 receptor could still function as a GPCR as ascertained by its capacity to mediate activation of Kir3.4 ST. This is shown in FIG. 10.

FIG. 10a shows typical TEVC recordings from oocytes expressing the specified constructs, and FIG. 10b summarizes these experiments (error bars represent sem; numbers next to bars indicate the number of experiments considered; single star indicates a statistically significant difference from 0 at p<0.01).

The effect of dopamine was reproduced with quinpirole, a stable agonist of dopaminergic receptors, and could be prevented by the antagonist sulpiride. This is shown in FIG. 11 with a typical TEVC recording from an oocyte expressing D2-K 0-25 where quinpirole (5 μM) induces a decrease in current which is reversed by sulpiride (5 μM).

Therefore, D2-K 0-25 appears as another GPCR-Kir6.2 fusion protein sensitive to agonists and antagonists, further validating the concept of GPCR-channel hybrid proteins. Our data are consistent with a purely mechanical transduction from the GPCR ligand binding site to the channel gate.

### Example 4

### Hybrid Protein Comprising Kir6.2 and β2 Adrenergic Receptor Deleted in its C-Terminus Part

As demonstrated above, the deletion of C-terminus part of the M2 membrane receptor fused to the N-terminus deleted Kir6.2 ion channel does not affect the electric signal generated by the hybrid protein consisting in full length M2 receptor fused to the N-terminus deleted Kir6.2 ion channel.

To confirm these results, new hybrid proteins comprising as membrane receptor the human β2 adrenergic receptor deleted in its C-terminus.

Two hybrid proteins have been constructed and correspond to the following constructions:
β2-Kir6.2Δ 62-25 Ha: β2 adrenergic receptor deleted of its 62 last amino acids is fused to Kir6.2 receptor which is deleted of the 25 amino acids in its N-terminus part and is deleted of its 36 amino acids in its C-Terminus part, and containing a Ha tag, and
β2-Kir6.2Δ 73-25 Ha: β2 adrenergic receptor deleted of its 73 last amino acids is fused to Kir6.2 receptor which is deleted of the 25 amino acids in its N-terminus part and is deleted of its 36 amino acids in its C-Terminus part, and containing a Ha tag.

The expression the β2-Kir6.2 constructs was achieved as mentioned in Example 1.

Cells expressing the constructs were stimulated with an agonist of the β2 receptor: Isoproterenol, at a final concentration 0.5 μM. The current variation was measured and the reaction was stopped by adding 3 mM Ba²⁺.

Results are shown in FIGS. 12 and 13.

Like the previous constructions, the fused β2 receptor is able to respond to stimulation and to generate an electric signal.

### Example 5

### Hybrid Protein Comprising Kir6.2 and CB1 Canabinoid Receptor Deleted or not in its C-Terminus Part

To extend the generalization of the membrane receptor-potassium channel, new constructs have been achieved by the Inventors.

CB 1 canabinoïd receptor has been fused to the Kir6.2 ion channel deleted in its N-terminus part. The constructions tested are the following ones:
CB1-Kir6.2Δ 0-25 Ha: CB1 canabinoïd receptor is fused to Kir6.2 receptor which is deleted of the 25 amino acids in its N-terminus part and is deleted of its 36 amino acids in its C-Terminus part, and containing a Ha tag, and
CB1-Kir6.2Δ 48-25 Ha: CB1 canabinoïd receptor deleted of its 48 last amino acids is fused to Kir6.2 receptor which is deleted of the 25 amino acids in its N-terminus part and is deleted of its 36 amino acids in its C-Terminus part, and containing a Ha tag.

The expression the CB1-Kir6.2 constructs was achieved as mentioned in Example 1.

Cells expressing the above constructs were stimulated with an agonist of the CB1 receptor: WIN 55, 212-2 (Sigma), at a final concentration 1 μM. The current variation was measured and the reaction was stopped by adding 3 mM Ba²⁺.

Results for CB1-Kir6.2Δ 0-25 Ha are shown in FIG. 14. CB1-Kir6.2Δ 48-25 Ha construct gives similar results.

Thus, another GPCR class A receptor, deleted or not in its C terminus part, is able to generate a potassium current when it is fused to a N-terminus deleted Kir6.2 ion channel.

### Example 6

### Hybrid Protein Comprising Kir6.2 and D3 Dopaminergic Receptor

Another GPCR receptor has been fused to Kir6.2 ion channel:

D3-KΔ 0-25 HA: D3 dopaminergic receptor is fused to Kir6.2 receptor which is deleted of the 25 amino acids in its N-terminus part and is deleted of its 36 amino acids in its C-Terminus part, and containing a Ha tag.

The expression the D3-Kir6.2 construct was achieved as mentioned in Example 1.

Cells expressing the above constructs were stimulated with dopamine, at a final concentration 0.3 μM.

The current variation was measured and the reaction was stopped by adding 3 mM Ba²⁺.

Results are shown in FIG. 15.

Again, a fusion between a class A GPCR and Kir6.2 deleted in its N-terminus part allows the generation of an electric flux, after stimulating the receptor.

### Example 7

### Hybrid Protein Comprising Kir6.2 and CCR2 Chemokine Receptor

Fusion protein consisting in CCR2 chemokine receptor has been fused to Kir6.2 deleted in its N-terminus part allows the generation of an electric flux, after stimulating the receptor by CCR2 agonist: CCL2 or MCP1, at a final concentration from about 0.3 μM to about at a final concentration 2 μM.

### Example 8

### Hybrid Protein Comprising Kir3.2 and M2 Muscarinic Receptor

In order to demonstrate that the above constructions fusing class A GPCR to Kir ion channel are not restricted to Kir6.2 ion channel, the hybrid protein M2−Kir3.2Δ 0-46 has been constructed.

By homology to the N-terminus Kir6.2 sequence, which is represented in FIG. 16, Kir3.2 deletion of 46 amino acids has demonstrated the same results as the deletion of 25 amino acids in Kir6.2: an hybrid protein M2−Kir3.2 0-46, when stimulated with Ach, is able to generate an electrical flux.

### Example 9

### Hybrid Protein Comprising Kv.1 and M2 Muscarinic Receptor

In order to demonstrate that the fusion can be generalized to potassium voltage dependant ion channel, a construction between M2 receptor and Kir1.1 ion channel has been made.

Kv1.1 ion channel is deleted in its N-terminus part of the 268 first amino acids, which corresponds to the 25 amino acids deleted in Kir6.2.

The M2−Kv1.1 0-268, when stimulated by Ach, is able to generate an electrical current.

Thus fusions between class A GPCR and K⁺ ion channels belonging to the Kir family or Kv family can serve as biomarquer for detecting the activity of class A GPCR receptors.

1-21. (canceled)

22. A hybrid protein comprising or consisting in
a. the sequence of a first membrane receptor, said first membrane receptor belonging to the G-protein coupled receptors (GPCR) class A family, covalently fused at its C-terminus to
b. the N-terminus sequence of an ion channel, said ion channel belonging to the potassium channel families selected from the inwardly rectifying potassium channels (Kir) family and the voltage-dependent potassium channels (Kᵥ) family,
said ion channel sequence being deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel,
said ion channel possibly containing a tag sequence,
said first membrane receptor being liable to present in its cytoplasmic tail at least one mutation, said cytoplasmic tail being a sequence delimited by the first amino acid after the last amino acid of the transmembrane helix and the last amino acid of said first membrane receptor,
a deletion of a number of amino acids ranging from 1 to the total number of amino acids of the region delimited by the cytoplasmic tail, and/or
an addition, of an additional sequence of a number of amino acids ranging from 1 to the total number of amino acids of the region delimited by the cytoplasmic tail, originating from a second membrane receptor different from said first membrane receptor, preferably said additional sequence corresponding to the C-terminus of said second membrane receptor and/or
a substitution of a number of amino acids ranging from 1 to the total number of amino acids of the region delimited by the cytoplasmic tail, with an substitute sequence of a number of amino acids ranging from 1 to the total number of amino acids of the region delimited by the cytoplasmic tail from a second membrane receptor different from said first membrane receptor
said hybrid protein being such that said ion channel retains the property of electrical current generation of said ion channel in its natural configuration, and that said first membrane receptor retains the ability to interact with the ligand of said first membrane receptor in its natural configuration.

23. The hybrid protein comprising the sequence of a first membrane receptor fused at its C-terminus to the N-terminus of a ion channel according to claim 22, wherein said first membrane receptor
a. is present in said hybrid protein in its natural configuration, or
b. is deleted of a number of amino acids ranging from 1 to the total number of amino acids of the region extending from the first amino acid at the N-terminus part of said ion channel to the first amino acids of the first α-helix of the transmembrane domain of said ion channel, or
c. has an addition, of an additional sequence of a number of amino acids ranging from 1 to the total number of amino acids of the region delimited by the cytoplasmic tail, originating from a second membrane receptor different from said first membrane receptor, preferably said additional sequence corresponding to the C-terminus of said second membrane receptor, or
d. has, a substitution of a number of amino acids ranging from 1 to the total number of amino acids of the region delimited by the cytoplasmic tail, with an substitute sequence of a number of amino acids ranging from 1 to the total number of amino acids of the region delimited by the cytoplasmic tail from a second membrane receptor different from said first membrane receptor,
and a linker is possibly present between the C-terminus of said first membrane receptor and the N-terminus part of said ion channel, said linker being absent in the natural configuration of said first membrane receptor and said ion channel.

24. The hybrid protein according to claim 22, comprising a linker present between the C-terminus of said first membrane receptor and the N-terminus part of said ion channel, said linker being absent in the natural configuration of said first membrane receptor and said ion channel, in particular comprising or constituted by six contiguous glycine residues, represented by the following sequence: -G-G-G-G-G-G- (SEQ ID NO 196).

25. The hybrid protein according to claim 22, comprising a tag, in particular chosen among the group consisting in SEQ ID NO 2q, q varying from 79 to 97.

26. The hybrid protein according to claim 22, wherein said ion channel is chosen among:
a. the Kir potassium channels selected from the group comprising the potassium channels Kir1.1, Kir2.1, Kir2.2, Kir2.3, Kir2.4, Kir3.1, Kir3.2, Kir3.3, Kir3.4, Kir4.1, Kir4.2, Kir5.1, Kir6.1, Kir6.2 and Kir7.1, or
b. the Kv potassium channels selected from the group comprising the potassium channels Kv1.1, Kv1.2, Kv1.3, Kv1.4, Kv1.5, Kv1.6, Kv1.7, Kv1.8, Kv2.1, Kv2.2, Kv3.1, Kv3.2, Kv3.3, Kv3.4, Kv4.1, Kv4.2, Kv4.3, Kv5.1, Kv6.1, Kv6.2, Kv6.3, Kv6.4, Kv7.1, Kv7.2, Kv7.3, Kv7.4, Kv7.5, Kv8.1, Kv8.2, Kv9.1, Kv9.2, Kv9.3, Kv10.1, Kv10.2, Kv11.1, Kv11.2, Kv11.3, Kv12.1, Kv12. and Kv12.3.

27. The hybrid protein according to claim 22, wherein said first and second membrane receptor sequence is the sequence of a membrane receptor belonging to the family of GPCR class A receptor chosen among the group comprising:
muscarinic receptor, in particular the human muscarinic M2 receptor, in particular comprising or constituted by SEQ ID NO 10,
adrenergic receptor, in particular the human β2-adrenergic receptor, in particular comprising or constituted by SEQ ID NO 12,
dopaminergic receptor, in particular the human dopaminergic long D2 receptor, in particular comprising or constituted by SEQ ID NO 14,
dopaminergic receptor, in particular the human dopaminergic D3 receptor, in particular comprising or constituted by SEQ ID NO 229
serotonergic receptor, in particular the human 5HT1αreceptor, in particular comprising or constituted by SEQ ID NO 16,
canabinoïd receptor, in particular the human CB1 receptor, in particular comprising or constituted by SEQ ID NO 230
CXCR4 receptor, in particular the human CXCR4 receptor, in particular comprising or constituted by SEQ ID NO 18, and
CCR5 receptor, in particular the human CCR5 receptor, in particular comprising or constituted by SEQ ID NO 20,
CCR2 receptor, in particular the human CCR2 receptor, in particular comprising or constituted by SEQ ID NO 231.

28. The hybrid protein according claim 22, wherein said ion channel is Kir6.2 protein,
said Kir6.2 being preferably chosen among the group comprising of:
the murine or human Kir6.2 ion channel in its natural configuration, and in particular comprising or being constituted by the amino acid sequence SEQ ID NO 2, or SEQ ID NO 6, or
the murine or human Kir6.2 ion channel deleted from 1 to 36 of its 36 last amino acids at the C-terminus, and in particular comprising or being constituted by the amino acid sequence SEQ ID NO 4 or SEQ ID NO 8.

29. The hybrid protein according to claim 22, wherein said hybrid protein is preferably chosen among the group consisting in SEQ ID NO 2q, q varying from 15 to 78 and from 99 to 103.

30. The hybrid protein according to claim 22, said hybrid protein being inserted in a membrane, preferably a membrane comprising lipids.

31. A nucleic acid molecule coding for the hybrid protein according to claim 22, in particular having a nucleic acid sequence chosen among the group consisting SEQ ID NO 2q-1, q varying from 15 to 78 and from 99 to 103, and comprising elements allowing the expression of said nucleic acid molecule in host cells such as among bacteria, yeast, mammals cells, insect cells or amphibian oocytes.

32. A method for in vitro diagnosis, in a biological sample of a subject, of pathologies associated with the presence or absence or the variation of amount of a molecule modifying the receptor activity of a first membrane receptor in its natural configuration,
said presence or absence or variation of amount of said molecule being assessed with respect to the presence or absence or the given amount of said molecule, in a sample isolated from an healthy subject, comprising:
contacting said hybrid protein, preferably said hybrid protein being a hybrid protein according to claim 22, preferably immobilized in a support, with a biological sample, said biological sample being liable to contain molecule being able to selectively interact with first membrane receptor part of said hybrid protein,
measuring the current generated by the ion channel part of the said hybrid protein, preferably measured by appropriate means of electrophysiology, or reconstitution in artificial lipid bilayers, or any techniques designed to measure ion flux through potassium channels,
comparing said current generated with the current generated with by said hybrid protein contacted with control sample, said control sample corresponding to sample either not containing said molecule, or containing a given amount of said molecule,
determining, from the previous steps, if the subject is afflicted by said pathologies,
said hybrid protein comprising or consisting in
a. the sequence of a first membrane receptor, said first membrane receptor belonging to the G-protein coupled receptors (GPCR) class A family, covalently fused at its C-terminus to
b. the N-terminus sequence of an ion channel, said ion channel belonging to the potassium channel families selected from the inwardly rectifying potassium channels (Kir) family and the voltage-dependent potassium channels (Kᵥ) family,
said ion channel sequence being deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel,
said ion channel possibly containing a tag sequence,
said first membrane receptor being liable to present in its cytoplasmic tail, said cytoplasmic tail being a sequence delimited by the first amino acid after the last amino acid of the transmembrane helix and the last amino acid of said first membrane receptor,
a deletion of a number of amino acids ranging from 1 to the total number of amino acids of the region delimited by the cytoplasmic tail, and/or
an addition, of an additional sequence of a number of amino acids ranging from 1 to the total number of amino acids of the region delimited by the cytoplasmic tail, originating from a second membrane receptor different from said first membrane receptor, preferably said additional sequence corresponding to the C-terminus of said second membrane receptor and/or
a substitution of a number of amino acids ranging from 1 to the total number of amino acids of the region delimited by the cytoplasmic tail, with an substitute sequence of a number of amino acids ranging from 1 to the total number of amino acids of the region delimited by the cytoplasmic tail from a second membrane receptor different from said first membrane receptor
said hybrid protein being such that said ion channel retains the property of electrical current generation of said ion channel in its natural configuration, and that said first membrane receptor retains the ability to interact with the ligand of said first membrane receptor in its natural configuration.

33. A method for screening a compound able to modify the activity of a first membrane receptor in its natural configuration, said compound being preferably an agonist or an antagonist,
comprising:
contacting a compound, with a hybrid protein, preferably said hybrid protein being a hybrid protein according to claim 22, preferably immobilized on a support, in presence of a ligand of said membrane receptor,
measuring the current generated by the ion channel of the said hybrid protein, preferably measured by appropriate means of electrophysiology, or reconstitution in artificial lipid bilayers, or any technique designed to directly or indirectly measure ion flux through potassium channels,
comparing said current generated with the current generated with by said hybrid protein contacted with an identified compound known to modify said receptor activity,
determining, from the previous steps the effect of said compound on the activity of said first membrane receptor in its natural configuration
said hybrid protein comprising or consisting in
a. the sequence of a first membrane receptor, said first membrane receptor belonging to the G-protein coupled receptors (GPCR) class A family, covalently fused at its C-terminus to
b. the N-terminus sequence of an ion channel, said ion channel belonging to the potassium channel families selected from the inwardly rectifying potassium channels (Kir) family and the voltage-dependent potassium channels (Kᵥ) family,
said ion channel sequence being deleted of a number of amino acids ranging from 1 to the total number of amino acids of the region extending from the first amino acid at the N-terminus part of said ion channel to the first amino acid of the cytoplasmic α-helix that precedes the first of the two transmembrane α-helices that form the pore region of said potassium channel,
said ion channel possibly containing a tag sequence,
said first membrane receptor being liable to present in its cytoplasmic tail, said cytoplasmic tail being a sequence delimited by the first amino acid after the last amino acid of the transmembrane helix and the last amino acid of said first membrane receptor,
a deletion of a number of amino acids ranging from 1 to the total number of amino acids of the region delimited by the cytoplasmic tail, and/or
an addition, of an additional sequence of a number of amino acids ranging from 1 to the total number of amino acids of the region delimited by the cytoplasmic tail, originating from a second membrane receptor different from said first membrane receptor, preferably said additional sequence corresponding to the C-terminus of said second membrane receptor and/or
a substitution of a number of amino acids ranging from 1 to the total number of amino acids of the region delimited by the cytoplasmic tail, with an substitute sequence of a number of amino acids ranging from 1 to the total number of amino acids of the region delimited by the cytoplasmic tail from a second membrane receptor different from said first membrane receptor
said hybrid protein being such that said ion channel retains the property of electrical current generation of said ion channel in its natural configuration, and that said first membrane receptor retains the ability to interact with the ligand of said first membrane receptor in its natural configuration.

34. The method according to claim 32, wherein said hybrid protein is immobilized on a support chosen among the group consisting in artificial membrane, natural or artificial membrane containing lipids, and chip comprising artificial membrane, or natural or artificial membrane containing lipids, or incorporated in the membrane of cells or liposomes.

35. The method according to claim 33, wherein said hybrid protein is immobilized on a support chosen among the group consisting in artificial membrane, natural or artificial membrane containing lipids, and chip comprising artificial membrane, or natural or artificial membrane containing lipids, or incorporated in the membrane of cells or liposomes.
